(19) United States
(12) Patent Application Publication
(10) Pub. No.: US 2015/0329638 A1
(43) Pub. Date: Nov. 19, 2015

(54) Use of DR6 Antagonists to Improve Motor Neuron Disease

(71) Applicant: BIOGEN IDEC MA INC., Cambridge, MA (US)

(72) Inventor: Sha MI, (US)

(73) Assignee: Biogen MA Inc., Cambridge, MA (US)

(21) Appl. No.: 14/655,126

(22) PCT Filed: Dec. 27, 2013
(86) PCT No.: PCT/US2013/078081

§ 371 Date: Jun. 24, 2015

Related U.S. Application Data
(60) Provisional application No. 61/747,108, filed on Dec. 28, 2012.

(51) Int. Cl. C07K 16/28; A61K 39/395; A61K 45/06

(52) U.S. Cl. CPC C07K 16/2878; A61K 45/06; A61K 39/3955; C07K 2317/56; C07K 2317/565; C07K 2317/567; C07K 2317/34; C07K 2317/76; C07K 2317/20; C07K 2317/21; C07K 2317/24; C07K 2317/55; C07K 2317/54; C07K 2317/622; C07K 2317/64; A61K 2039/505

(57) ABSTRACT

The present invention relates to Death Receptor-6 (DR6) antagonists and methods of their use in improving motor neuron disease. Novel affinity enhanced anti-DR6 antibodies are also provided. The invention also pertains to methods of identifying additional anti-DR6 antagonists.

### REFERENCE TO A SEQUENCE LISTING SUBMITTED ELECTRONICALLY VIA EFS-WEB

The content of the electronically submitted sequence listing (Name: 215939PC01_Sequence_Listing.txt, Size: 127,039 bytes, and Date of Creation: Dec. 3, 2013) is herein incorporated by reference in its entirety.

### BACKGROUND OF THE INVENTION

Apoptosis (i.e., programmed cell death) has been shown to play an important role in numerous diseases of the nervous system including both acute and chronic injuries. For example, the role of apoptosis has been demonstrated in Alzheimer's disease, Parkinson's disease, Huntington's disease, motor neuron disease (e.g., amyotrophic lateral sclerosis, which is also called ALS or Lou Gehrig's disease), multiple sclerosis, neuronal trauma and cerebral ischemia (e.g., stroke).

Many studies have been directed to understanding the molecular mechanisms of apoptosis, and these studies have led to the discovery of a family of receptors called the death receptors. Eight death receptors, which are characterized by a cytoplasmic death domain, have been identified thus far. The death receptors have been grouped into two different families. Members of the first family recruit a death inducing signaling complex (DISC), which promotes apoptotic signaling. Members of the second family recruit a different set of molecules to transduce apoptotic signals. Interestingly, members of the second family also transduce cell survival signals.

Death receptor 6 (DR6) is a member of the second family of death receptors. DR6 is widely expressed, but appears to function differently in different cell types. DR6 mRNA has been observed in heart, brain, placental, pancreas, lymph node, thymus, and prostate tissues. Lower levels have been observed in other cell types including skeletal muscle, kidney, and testes, but little or no expression has previously been observed in adult liver or any lines of hematopoeitic origin. Interestingly, it has been observed that DR6 is capable of inducing apoptosis in only a subset of cells tested. For example, overexpression of DR6 in HeLa S3 cervical carcinoma cells resulted in apoptosis in a death-domain-dependent manner (Pan et al. FEBS 431:351-356 (1998)). In contrast, DR6 did not induce cell death in MCF7 (a human breast adenocarcinoma line) cells (Pan et al. FEBS 431:351-356 (1998)). In addition, Nikoleav et al. (Nature 457:981-990 (2009)) have shown that beta-amyloid precursor protein (APP) is a DR6 ligand and suggested that the binding of an APP fragment to DR6 triggers degeneration of neuronal cell bodies and axons. The interaction of DR6 with p75 is also thought to promote apoptosis (WO 2010/062904).

Drugs that can specifically modulate apoptosis may be useful for treating diseases involving neuronal cell death, in particular because neurons may have less capacity to regenerate than other cell types. To date, DR6 antagonists have not been shown to be capable of treating ongoing motor neuron disease in adult subjects. The identification of DR6 antagonists which are useful in treatment of motor neuron disease would be of great benefit.

### BRIEF SUMMARY OF THE INVENTION

Overexpression of DR6 has been associated with cortical neuron cell death. As demonstrated herein, DR6 is upregulated during the course of motor neuron disease in human ALS as well as in animal models of ALS (e.g., the SOD1^{G93A} mouse model). The data presented herein demonstrate that DR6 antagonists can be used, e.g., to improve the course of motor neuron disease, for example by promoting the preservation of neuromuscular junctions. In one embodiment, the subject antagonists can promote functional survival in, e.g., ALS by promoting motor neuron survival and remyelination through Schwann cells and dorsal root ganglion (DRG) neurons. More specifically, in a model of ALS, DR6 antagonists improve the course of disease even when administered in the early phase of ALS after motor neuron termini have begun to retract from muscle cells, i.e., after reduced muscle innervation can be demonstrated. In one embodiment, the DR6 antagonist is administered before DR6 expression (e.g., as measured by increased mRNA and/or increased protein) is upregulated in motor neurons. In one embodiment, the DR6 antagonist is administered after DR6 expression is upregulated in motor neurons. In another embodiment, the DR6 antagonist is administered after the disease has become symptomatic. The working examples of this application demonstrate that DR6 antagonists promote survival of adult motor neuron cells and increase axon outgrowth in vivo and/or in vitro. DR6 antagonists also promote axon integrity in motor neuron cocultures, decrease the number of pathogenic axons, and preserve neuromuscular junctions. In another embodiment, DR6 antagonists are used to reduce neuropathic pain.

Furthermore, in one embodiment, antagonists of DR6 and p75, including anti-DR6 antibodies (including antigen binding fragments thereof, e.g., Fab fragments, as well as antibodies or fragments that are modified, e.g., by engineering or conjugation (e.g., by attachment of a moiety such as PEG)), antagonistic DR6 nucleic acid molecules (such as antisense molecules aptamers, or RNAi), and DR6-Fc fusion protein are able to inhibit the formation of a complex between DR6 and p75 (e.g., by specifically blocking the binding of DR6 to p75 or by blocking the dimerization of DR6) and to inhibit death of cells of the nervous system. Accordingly, antagonists of DR6 and/or p75 can be useful for therapy in ongoing motor neuron disease.

In one embodiment, a DR6 antagonist is an anti-DR6 antibody, e.g., an isolated antibody or antigen-binding fragment thereof that can specifically bind to a DR6 polypeptide. In some embodiments, the DR6 antibody inhibits formation of a complex between DR6 and p75. In some embodiments, the DR6 antibody inhibits binding of DR6 to p75. In some embodiments, the DR6 antibody inhibits binding of DR6 to p75 but does not inhibit binding of DR6 to beta-amyloid precursor protein (APP). In one embodiment, the anti-DR6 antibody binds with high affinity to human, cynomologous, and rat DR6, e.g., with an EC50 of 1 nM or less.

Several anti-DR6 antibodies are known in the art and can be used in the methods of the instant invention (see, e.g., WO 2008/080045). In one embodiment, an anti-DR6 antibody is one which blocks the formation of a complex between DR6 and p75 and does not block the binding of APP to p75. In one embodiment, an anti-DR6 antibody is one which blocks the formation of a complex between DR6 and p75, does not block the binding of APP to p75, and which does not comprise all six CDRs of the 5D10 antibody.

In some embodiments, the DR6 antibody is an isolated antibody or fragment thereof that specifically binds to DR6, wherein the VL of said antibody or fragment thereof comprises the amino acid sequence of SEQ ID NO: 167.

In some embodiments, the DR6 antibody is an isolated antibody or fragment thereof that specifically binds to DR6, wherein the VH and VL of said antibody or fragment thereof comprise, respectively, the amino acid sequences of SEQ ID NO: 127 and SEQ ID NO:167.

In one embodiment, the VL-CDR3 comprises the amino acid sequence of SEQ ID NO:168.

In some embodiments, the DR6 antibody is an isolated antibody or fragment thereof that specifically binds to DR6, wherein the VL of said antibody or fragment thereof comprises VL-CDR1, VL-CDR2, and VL-CDR3 amino acid sequences of SEQ ID NOs: 133, 134, and 168.

In some embodiments, a DR6 antibody or fragment thereof that specifically binds to DR6 comprises a VL that comprises the VL-CDR1, VL-CDR2, and VL-CDR3 amino acid sequences of SEQ ID NOs: 133, 134, and 168 and a VH that comprises the VH-CDR1, VH-CDR2, and VH-CDR3 amino acid sequences of SEQ ID NOs: 128, 129, and 130.

In various embodiments of the above-described antibodies or fragments thereof, the VH framework regions and/or VL framework regions are human, except for five or fewer amino acid substitutions.

In various embodiments of the above-described antibodies or fragments thereof, the heavy and light chain variable domains are murine. In further embodiments, the heavy (SEQ ID NO:127) and light chain (SEQ ID NO:167) variable domains are from 5D10Y93A (“Y93A”).

In various embodiments, the above-described antibodies or fragments thereof are humanized, chimeric, primatized, or fully human.

In certain embodiments, the above-described antibodies or fragments thereof are Fab fragments, Fab′ fragments, F(ab)₂ fragments, or Fv fragments. In certain embodiments, the above-described antibodies are single chain antibodies. In certain embodiments, the antibodies or fragments thereof are conjugated to a polymer. In certain embodiments, the polymer is a polyalkylene glycol, e.g., polyethylene glycol (PEG).

In certain embodiments, the above-described antibodies or fragments thereof comprise light chain constant regions selected from the group consisting of a human kappa constant region and a human lambda constant region.

In certain embodiments, the above-described antibodies or fragments thereof comprise a heavy chain constant region or fragment thereof. In further embodiments, the heavy chain constant region or fragment thereof is derived from a wild-type immunoglobulin, e.g., human IgG1 or IgG4. In another embodiment, a constant region has reduced effector function as compared to a wild type constant region, e.g., IgG4agly (e.g., having a mutation at position 299 of T to another amino acid, e.g., A or K), IgG1agly (e.g., having a mutation at position 299 of T to another amino acid, e.g., A or K), a chimeric IgG4/IgG2 FC (Armour and Clark deltaB hybrid as disclosed in Eur. J. Immunol. 1999. 29:2613) or a chimeric IgG4Pagly/IgG1 hybrid (see, e.g., US 2012/0100140 and US 2008/0063635).

In one embodiment, the invention pertains to a method of identifying DR6 antagonists that do not cause cell death using a non-neural cell line. For example, in one embodiment, a non-neural cell line such as HEK 293 cells or Jurkat cells is transfected with DR6 or with a DR6-FAS chimeric molecule

DR6 binding moieties (e.g., test antibodies) are then tested for their ability to act as agonists or antagonists. Apoptosis can be measured or LDH release can be measured and used as a surrogate for cell death. If a test antibody cross-links DR6, thereby triggering FAS and JNK activation, cell death (or LDH release) occurs. If the antibody does not crosslink DR6, no cell death (or LDH release) occurs. Using such an assay, DR6 antagonists that do not crosslink DR6 and do not cause cell death can be selected.

The therapeutic methods described herein relate generally to methods of promoting survival and preventing apoptosis of motor neuron cells of the adult nervous system. In certain embodiments, the methods include a method of promoting survival of motor neuron cells of the nervous system comprising contacting said cells with a DR6 antagonist. The step of contacting can be performed in vivo, e.g., by systemic administration of a DR6 antagonist or by local (e.g., intrathecal) administration of a DR6 antagonist. In particular, the subject methods are useful in improving the course of motor neuron disease (e.g. amyotrophic lateral sclerosis, which is also called ALS or Lou Gehrig's disease as well as other motor neuron diseases, such as spinal muscular atrophy (SMA) (e.g., types 0-4) or other diseases or disorders associated with motor neuron disease or, e.g., neuropathic pain.

In one embodiment, a DR6 antagonist can be used in combination with a p75 antagonist. The p75 antagonist can be used simultaneously or sequentially.

### BRIEF DESCRIPTION OF THE DRAWINGS/FIGURES

FIG. 1 DR6 mRNA and protein level are up-regulated in spinal cord of SOD1^{G93A} mice and human ALS patients. (A) In situ hybridization of DR6 positive motor neurons in SOD1^{G93A} and normal mice (at the age of day 65). (B) Quantification of (A). (C) The brain lysates of WT and DR6-null mice (day 30) were subjected to Western Blot analysis to test DR6 antibody specificity. β-actin was used as an internal control. (D) Quantitation of Western Blot analysis of SOD1^{G93A} and normal mice spinal cord (day 100) for DR6 expression. β-actin was used as an internal control. The Western blot signals were quantified by densitometry. The plot was presented as the ratio of DR6 over actin. (E) Quantification of in situ hybridization of DR6 positive neurons in human post-mortem spinal cord tissues. (F) Western Blot analysis of human post-mortem spinal cord samples for the expression of DR6. β-actin was used as an internal control. (5 ALS samples vs. 2 normal samples). (G) Quantification of (F) by densitometry. The plot was presented as the ratio of DR6 over actin.

FIG. 2 Anti-DR6 antibody promotes human motor neuron survival and preserves axon integrity in vitro. (A) Representative ICC images of ES-cell derived human motor neuron for DR6 expression, DR6 (green), NF (red). (B) Representative ICC images of human motor neuron survival by growth factor removal, NF (green). (C) Quantification of surviving motor neurons number in (B). (D) Quantification of axon length in (B). (E) Representative ICC images of human motor neuron survival by sodium arsenite, NF (green). (F) Quantification of surviving motor neurons number in (E). (G) Quantification of axon length in (E). (H) Quantification by MSD analysis of NF level in ICC images of rat motor neuron survival co-cultured with purified astrocyte from either normal or SOD1^{G93A} mice. (I) Quantification of axon beading in ICC images of rat motor neuron survival co-cultured with purified astrocyte from either normal or SOD1^{G93A} mice. (J) Western blot analysis for cleaved caspase 3, phosphorylated Akt in human motor neuron. β-actin and total Akt were used as internal controls. (K) Quantification of (J) by densitometry.

FIG. 3 Blocking DR6 promote survival and functional recovery in SOD1^{G93A} mice. (A) Time-to-event analysis for disease onset, the age at which mice showed slightly impaired initiation of movement (20 males and 20 females/group). (B) Time-to-event analysis for survival, the age at which mice were unable to right itself within 30 s when placed on either or both side(s) (20 males and 20 females/group). (C) Body weight analysis of control and 5D10 treated SOD1^{G93A} mice (10 males and 10 females/group). (D) Rota-rod analysis of control and 5D10 treated SOD1^{G93A} mice, data presented as latency to fall (S) (10 males and 10 females/group).

FIG. 4 Blocking DR6 promotes tissue integrity in SOD1^{G93A} mice. (A) Quantification of NMJs in IHC images taken of gastrocnemius muscle in control or anti-DR6 antibody treated SOD1^{G93A} mice (day 100, n=6/group). (B) littermate match analysis of gastrocnemius muscle NMJs. (C) Quantification of NMJs in IHC images taken of diaphragm NMJs in control or anti-DR6 antibody treated SOD1^{G93A} mice (day 100, n=6/group). (D) Littermate match analysis of diaphragm NMJs. (E) Quantification of gastrocnemius muscle NMJs in SOD1^{G93A}/DR6−/− and SOD1^{G93A}/DR6+/+ mice (day 60, n=4/group). (F) Littermate match analysis of NMJs in (E). (G) Representative images of Nissl-stained lumbar spinal cord motor neurons in control or 5D10 treated SOD1^{G93A} mice (day 80, n=3/group, 3 sections/animal). (H) Quantification of lumbar spinal cord motor neuron counts/section in (G). (I) Quantification of GFAP staining in images of lumbar spinal cord of control or 5D10 treated SOD1^{G93A} mice (day 60, n=3/group, 3 sections/animal). Data are presented as mean fluorescence intensity (MFI). (J) Representative images of toluidine blue staining of sciatic nerve axons in control or 5D10 treated SOD1^{G93A} mice (day 100, n=3/group, 3 sections/animal). (K) Quantification of sciatic nerve pathological axons in (J).

FIG. 5. Anti-DR6 antibodies 5D10 and M53-E04 bind to human DR6 (panel A). E04 mAb leads to cell death, whereas 5D10 promotes cell survival (panel B).

FIG. 6. 5D10Y93A and 5D10Y57A have improved affinity for rat DR6.

### DETAILED DESCRIPTION OF THE INVENTION

### Definitions

Unless defined otherwise, all technical and scientific terms used herein have the same meaning as commonly understood by one of ordinary skill in the art to which this invention belongs. In case of conflict, the present application including the definitions will control. Unless otherwise required by context, singular terms shall include pluralities and plural terms shall include the singular. Although methods and materials similar or equivalent to those described herein can be used in practice or testing of the methods described herein, suitable methods and materials are described below. The materials, methods and examples are illustrative only and are not intended to be limiting. Other features and advantages of the antibodies and methods described herein will be apparent from the detailed description and from the claims. In order to further define this invention, the following terms and definitions are provided.

It is to be noted that the term “a” or “an” entity, refers to one or more of that entity; for example, “an immunoglobulin molecule,” is understood to represent one or more immunoglobulin molecules. As such, the terms “a” (or “an”), “one or more,” and “at least one” can be used interchangeably herein.

As used herein, a “therapeutically effective amount” refers to an amount effective, at dosages and for periods of time necessary, to achieve a desired therapeutic result. A therapeutic result can be, e.g., lessening of symptoms, prolonged survival, improved mobility, or the like. A “therapeutically effective amount” can achieve any one of the desired therapeutic results or any combination of multiple desired therapeutic results. A therapeutic result need not be a “cure.” In one embodiment, DR6 antagonists improve the course of disease even when administered in the early phase of ALS after motor neuron termini have begun to retract from muscle cells, i.e., after muscle innervation can be demonstrated. In one embodiment, the DR6 antagonist is administered after DR6 expression is upregulated in motor neurons. In another embodiment, the DR6 antagonist is administered after the disease has become symptomatic. In yet another embodiment, the DR6 antagonist is administered to preserve neuromuscular junctions.

As used herein, a “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

As used herein, a “polynucleotide” can contain the nucleotide sequence of the full length cDNA sequence, including the untranslated 5′ and 3′ sequences, the coding sequences, as well as fragments, epitopes, domains, and variants of the nucleic acid sequence. The polynucleotide can be composed of any polyribonucleotide or polydeoxyribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotides can be composed of triple-stranded regions comprising RNA or DNA or both RNA and DNA. Polynucleotides can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

A polypeptide can be composed of amino acids joined to each other by peptide bonds or modified peptide bonds, i.e., peptide isosteres, and can contain amino acids other than the 20 gene-encoded amino acids (e.g. non-naturally occurring amino acids). The polypeptides described herein can be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification can be present in the same or varying degrees at several sites in a given polypeptide. Also, a given polypeptide can contain many types of modifications. Polypeptides can be branched, for example, as a result of ubiquitination, and they can be cyclic, with or without branching. Cyclic, branched, and branched cyclic polypeptides can result from posttranslation natural processes or can be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, 2nd Ed., T. E. Creighton, W.H. Freeman and Company, New York (1993); Posttranslational Covalent Modification of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene can be endogenous or exogenous to the organism, present integrated into a chromosome or present in a transfection vector that is not integrated into the genome. The expression of the gene is either completely or partially inhibited. RNAi can also be considered to inhibit the function of a target RNA; the function of the target RNA can be complete or partial.

The term “aptamer” as used herein refers to non-antibody molecules that bind to a specific target, e.g., oligonucleotide aptamers or peptide aptamers. See, e.g., “Cell-Specific Aptamers as Emerging Therapeutics” Journal of Nucleic Acids (2011) 1-18.

As used herein, the term “antisense” refers to single strands of DNA or RNA that are complementary to a chosen sequence. In the case of antisense RNA, they prevent protein translation of certain messenger RNA strands by binding to them. Antisense DNA can be used to target a specific, complementary (coding or non-coding) RNA. If binding takes place, the DNA/RNA hybrid can be degraded by the enzyme RNase H.

The terms “percent sequence identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, which is publicly available. Another suitable program is MUSCLE, which is also publicly available. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, which is available, e.g., from the EBI. It will also be appreciated that the final alignment used to calculated percent sequence identity can be curated either automatically or manually.

The terms “fragment,” “variant,” “derivative” and “analog” when referring to a Death Receptor-6 (DR6) antagonist include altered antagonist molecules which promote nervous system cell survival. For example, soluble DR6 polypeptides can include DR6 proteolytic fragments, deletion fragments and in particular, fragments which more easily reach the site of action when delivered to an animal. Soluble DR6 polypeptides can comprise variant DR6 regions, including fragments as described above, and also polypeptides with altered amino acid sequences owing to amino acid substitutions, deletions, or insertions. Variants can occur naturally, such as an allelic variant. By an “allelic variant” is intended to include alternate forms of a gene occupying a given locus on a chromosome of an organism. Genes II, Lewin, B., ed., John Wiley & Sons, New York (1985). Non-naturally occurring variants can be produced using art-known mutagenesis techniques. Soluble DR6 polypeptides can comprise conservative or non-conservative amino acid substitutions, deletions or additions. DR6 antagonists can also include derivative molecules. For example, soluble DR6 polypeptides can include DR6 regions which have been altered so as to exhibit additional features not found on the native polypeptide. Examples include fusion proteins and protein conjugates.

A “polypeptide fragment” refers to a short amino acid sequence of a DR6 polypeptide. Protein fragments can be “free-standing,” or comprised within a larger polypeptide of which the fragment forms a part of region. In one embodiment a fragment of DR6 is a soluble form of the molecule which lacks the transmembrand domain. Such soluble forms of DR6 can be used as antagonists. Representative examples of polypeptide fragments, include, for example, fragments comprising about 5 amino acids, about 10 amino acids, about 15 amino acids, about 20 amino acids, about 30 amino acids, about 40 amino acids, about 50 amino acids, about 60 amino acids, about 70 amino acids, about 80 amino acids, about 90 amino acids, and about 100 amino acids in length.

As used herein, the term “antigen binding molecule” (“ABM”) refers in its broadest sense to a molecule that specifically binds an antigenic determinant. It is understood by those of skill in the art that fragments of mature antibodies can bind specifically to an antigen. Accordingly, an antigen binding molecule, as the term is used herein, includes, but is not limited to, fragments of mature antibodies that bind specifically to a target antigen. An ABM need not contain a constant region. If one or more constant region(s) is present, in particular embodiments, the constant region is substantially identical to human immunoglobulin constant regions, e.g., at least about 85-90%, or about 95% or more identical.

In one embodiment, the DR6 antagonists are “antibody” or “immunoglobulin” molecules, or antigen-binding fragments thereof, e.g., naturally occurring antibody or immunoglobulin molecules or engineered antibody molecules or fragments that bind antigen in a manner similar to antibody molecules. The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). In one embodiment, in lieu of an antibody molecule, an antigen binding fragment of an antibody molecule can be used. Exemplary antigen binding fragments of antibody molecules include those set forth in “Antibody Fragments: Hope and Hype” mAbs 2010 2:77-83.

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table 1 as a comparison.

TABLE 1
CDR DEFINITIONS¹

| | Kabat | Chothia |
|---|---|---|
| V_{H} CDR1 | 31-35 | 26-32 |
| V_{H} CDR2 | 50-65 | 52-58 |
| V_{H} CDR3 | 95-102 | 95-102 |
| V_{L} CDR1 | 24-34 | 26-32 |
| V_{L} CDR2 | 50-56 | 50-52 |
| V_{L} CDR3 | 89-97 | 91-96 |

¹Numbering of all CDR definitions in Table 1 is according to the numbering conventions set forth by Kabat et al. (see below).

The exact residue numbers which encompass a particular CDR will vary depending on the sequence and size of the CDR. Those skilled in the art can routinely determine which residues comprise a particular CDR given the variable region amino acid sequence of the antibody.

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in a DR6 antibody or antigen-binding fragment, variant, or derivative thereof are according to the Kabat numbering system.

In one embodiment, an antigen binding molecule comprises at least one heavy or light chain CDR of an antibody molecule. In another embodiment, an antigen binding molecule comprises at least two CDRs from one or more antibody molecules. In another embodiment, an antigen binding molecule comprises at least three CDRs from one or more antibody molecules. In another embodiment, an antigen binding molecule comprises at least four CDRs from one or more antibody molecules. In another embodiment, an antigen binding molecule comprises at least five CDRs from one or more antibody molecules. In another embodiment, an antigen binding molecule comprises at least six CDRs from one or more antibody molecules. Exemplary antibody molecules comprising at least one CDR that can be included in the subject antigen binding molecules are known in the art and exemplary molecules are described herein.

Antibodies or antigen-binding fragments thereof include, but are not limited to, polyclonal, monoclonal, multispecific, human, humanized, primatized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a V_{L} or V_{H} domain, fragments produced by a Fab expression library, and anti-idiotypic (anti-Id) antibodies (including, e.g., anti-Id antibodies to binding molecules disclosed herein). ScFv molecules are known in the art and are described, e.g., in U.S. Pat. No. 5,892,019. Immunoglobulin or antibody molecules can be of any type (e.g., IgG, IgE, IgM, IgD, IgA, and IgY), class (e.g., IgG₁, IgG₂, IgG₃, IgG₄, IgA₁ and IgA₂) or subclass of immunoglobulin molecule.

Antibody fragments, including single-chain antibodies, can comprise the variable region(s) alone or in combination with the entirety or a portion of the following: hinge region, C_{H}1, C_{H}2, and C_{H}3 domains. Antigen-binding fragments can also comprise any combination of variable region(s) with a hinge region, C_{H}1, C_{H}2, and C_{H}3 domains. Antibodies or antigen-binding fragments thereof can be from any animal origin including birds and mammals. In certain embodiments, the antibodies are human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al.

As used herein, the term “heavy chain portion” includes amino acid sequences derived from an immunoglobulin heavy chain. A polypeptide comprising a heavy chain portion comprises at least one of: a C_{H}1 domain, a hinge (e.g., upper, middle, and/or lower hinge region) domain, a C_{H}2 domain, a C_{H}3 domain, or a variant or fragment thereof. For example, a binding polypeptide can comprise a polypeptide chain comprising a C_{H}1 domain; a polypeptide chain comprising a C_{H}1 domain, at least a portion of a hinge domain, and a C_{H}2 domain; a polypeptide chain comprising a C_{H}1 domain and a C_{H}3 domain; a polypeptide chain comprising a C_{H}1 domain, at least a portion of a hinge domain, and a C_{H}3 domain, or a polypeptide chain comprising a C_{H}1 domain, at least a portion of a hinge domain, a C_{H}2 domain, and a C_{H}3 domain. In another embodiment, a polypeptide comprises a polypeptide chain comprising a C_{H}3 domain. Further, a binding polypeptide can lack at least a portion of a C_{H}2 domain (e.g., all or part of a C_{H}2 domain). As set forth above, it will be understood by one of ordinary skill in the art that these domains (e.g., the heavy chain portions) can be modified such that they vary in amino acid sequence from the naturally occurring immunoglobulin molecule.

The heavy chain portions of a binding polypeptide can be derived from different immunoglobulin molecules. For example, a heavy chain portion of a polypeptide can comprise a C_{H}1 domain derived from an IgG1 molecule and a hinge region derived from an IgG₃ molecule. In another example, a heavy chain portion can comprise a hinge region derived, in part, from an IgG₁ molecule and, in part, from an IgG₃ molecule. In another example, a heavy chain portion can comprise a chimeric hinge derived, in part, from an IgG₁ molecule and, in part, from an IgG₄ molecule.

As used herein, the term “light chain portion” includes amino acid sequences derived from an immunoglobulin light chain. Typically, the light chain portion comprises at least one of a V_{L} or C_{L} domain.

An isolated nucleic acid molecule encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations can be introduced by standard techniques, such as site-directed mutagenesis and PCR-mediated mutagenesis. For example, conservative amino acid substitutions are made at one or more non-essential amino acid residues.

Antibodies or antigen-binding fragments thereof can act as antagonists of DR6 as described herein. For example, an antibody can function as an antagonist by blocking or inhibiting the suppressive activity of the DR6 polypeptide.

As used herein, the term “engineered antibody” refers to an antibody in which the variable domain in either the heavy and light chain or both is altered by at least partial replacement of one or more CDRs from an antibody of known specificity and, if necessary, by partial framework region replacement and sequence changing. Although the CDRs can be derived from an antibody of the same class or even subclass as the antibody from which the framework regions are derived, it is envisaged that the CDRs will be derived from an antibody of different class and/or an antibody from a different species. An engineered antibody in which one or more “donor” CDRs from a non-human antibody of known specificity is grafted into a human heavy or light chain framework region is referred to herein as a “humanized antibody.” In some cases it is not necessary to replace all of the CDRs with the complete CDRs from the donor variable region to transfer the antigen binding capacity of one variable domain to another. Rather, in some cases, it is only be necessary to transfer those residues that are necessary to maintain the activity of the target binding site. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein, the term humanized is used to refer to an antigen-binding molecule derived from a non-human antigen-binding molecule, for example, a murine antibody, that retains or substantially retains the antigen-binding properties of the parent molecule but which is less immunogenic in humans. This can be achieved by various methods including (a) grafting the entire non-human variable domains onto human constant regions to generate chimeric antibodies, (b) grafting only the non-human CDRs onto human framework and constant regions with or without retention of critical framework residues (e.g., those that are important for retaining good antigen binding affinity or antibody functions), or (c) transplanting the entire non-human variable domains, but “cloaking” them with a human-like section by replacement of surface residues. Such methods are disclosed in Jones et al., Morrison et al., Proc. Natl. Acad. Sci., 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31(3):169-217 (1994), all of which are incorporated by reference in their entirety herein. There are generally 3 complementarity determining regions, or CDRs, (CDR1, CDR2 and CDR3) in each of the heavy and light chain variable domains of an antibody, which are flanked by four framework subregions (i.e., FR1, FR2, FR3, and FR4) in each of the heavy and light chain variable domains of an antibody: FR1-CDR1-FR2-CDR2-FR3-CDR3-FR4. A discussion of humanized antibodies can be found, inter alia, in U.S. Pat. No. 6,632,927, and in published U.S. Application No. 2003/0175269, both of which are incorporated herein by reference in their entirety.

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more open reading frames (ORFs) to form a continuous longer ORF, in a manner that maintains the correct reading frame of the original ORFs. Thus, the resulting recombinant fusion protein is a single protein containing two ore more segments that correspond to polypeptides encoded by the original ORFs (which segments are not normally so joined in nature.) Although the reading frame is thus made continuous throughout the fused segments, the segments can be physically or spatially separated by, for example, in-frame linker sequence.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (shRNA), small interfering RNA (siRNA) or any other RNA product and the translation of such mRNA into polypeptide(s). If the final desired product is biochemical, expression includes the creation of that biochemical and any precursors.

By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as guinea pigs, rabbits, rats, mice; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; bears; and so on. In certain embodiments, the mammal is a human subject.

### Death Receptor-6 (DR6/TNFRSF21)

DR6 is expressed in cells of the nervous system including neurons and oligodendrocyte precursor cells and that DR6 can induce cell death in these cells.

DR6 is a polypeptide consisting of 655 amino acids. In certain embodiments, the human polypeptide is encoded by an mRNA comprising the nucleotides of SEQ ID NO:1 (Accession Number: NM_{—}014452).

TABLE 2

| Domain or Region | Beginning Residue | Ending Residue |
|---|---|---|
| Signal Sequence | 1 | 40 or 41 |
| Extracellular Domain | 41 or 42 | 349 or 350 |
| TNFR-like Cysteine-Rich Motif-1 | 50 | 88 |
| TNFR-like Cysteine-Rich Motif-2 | 90 | 131 |
| TNFR-like Cysteine-Rich Motif-3 | 133 | 167 |
| TNFR-like Cysteine-Rich Motif-4 | 170 | 211 |
| Transmembrane | 350 or 351 | 367-370 |
| Cytoplasmic | 368-371 | 655 |
| Death Domain | 415 | 498 |
| Leucine Zipper Motif | 497 | 526 |

In certain embodiments, the human DR6 polypeptide sequence comprises the amino acids of SEQ ID NO:2 (Accession Number: 075509). In certain embodiments, mouse DR6 is encoded by an mRNA comprising the nucleotides of SEQ ID NO:3 (Accession Number: NM_{—}178589). In certain embodiments, the mouse DR6 polypeptide sequence comprises the amino acid sequence of SEQ ID NO:4 (Accession Number: NP_{—}848704). In certain embodiments, rat DR6 is encoded by an mRNA comprising the nucleotides of SEQ ID NO: 169 (Accession Number: NM_{—}001108207) and rat DR6 polypeptide comprises the amino acid sequence of SEQ ID NO: 170 (Accession Number: NP_{—}001101677).

Table 2 lists DR6 domains and other regions according to the amino acid residue number based on the sequence of SEQ ID NO:2. As one of skill in the art will appreciate, the beginning and ending residues of the domains listed below can vary depending upon the computer modeling program used, the method used for determining the domain, minor sequence variations etc.

### P75/TNR16

It has also been discovered that p75 neurotrophin receptor is a ligand for DR6. P75, also known as tumor necrosis factor receptor superfamily member 16 (TNR16 or TNFRSF16) or nerve growth factor receptor (NGFR), is a polypeptide consisting of 427 amino acids. The human polypeptide sequence is Accession Number NP_{—}002498 (SEQ ID NO: 165) and the nucleic acid sequence is Accession Number NM_{—}002507 (SEQ ID NO: 166). The p75 protein, like the DR6 protein, includes an extracellular region containing four TNFR Cysteine-Rich motifs, a transmembrane region, and an intracellular region containing a death domain. It has previously been shown that p75 is a low affinity receptor which can bind to NGF, BDNF, NT-3, and NT-4. Mi et al. Nat. Neuroscience 7:221-228 (2004). In addition, p75 is a component of the LINGO-1/Nogo-66 receptor signaling pathway and can mediate survival and death of neuronal cells. Id.

### Antagonists of DR6 and p75

A method for promoting survival of cells of the nervous system comprises contacting said cells with a DR6 antagonist. In another embodiment, methods for promoting oligodendrocyte proliferation, differentiation or survival comprise contacting oligodendrocyte cells or oligodendrocyte precursor cells with a DR6 antagonist. Another embodiment provides methods for promoting myelination comprising contacting a mixture of neuronal cells and oligodendrocytes or oligodendrocyte precursor cells with a DR6 antagonist. Yet another embodiment provides methods of inhibiting the formation of a complex between DR6 and p75 comprising contacting a DR6 polypeptide and/or a p75 polypeptide with a DR6 antagonist under conditions wherein the formation of a complex of DR6 and p75 is inhibited. Similarly, the methods described herein also include methods of inhibiting the binding of DR6 to p75 comprising contacting a DR6 polypeptide and/or a p75 polypeptide with a p75 antagonist.

Antagonists of DR6 and/or p75 and methods of using such antagonists have also been provided in International Publication No. WO 2010/062904 and U.S. Provisional Appl. No. 61/117,917 (filed Nov. 25, 2008), each of which is herein incorporated by reference in its entirety.

A DR6 antagonist can be a DR6 antagonist polypeptide (e.g., a DR6 Fc molecule), a DR6 antibody, a DR6 antagonist polynucleotide, a DR6 aptamer, or a combination of two or more DR6 antagonists. Additional embodiments include methods for treating a condition associated with death of cells of the nervous system comprising administering a therapeutically effective amount of a DR6 antagonist.

A p75 antagonist can be a p75 antagonist polypeptide, a p75 antagonist compound, a p75 antibody, a p75 antagonist polynucleotide, a p75 aptamer, or a combination of two or more p75 antagonists. Additional embodiments include methods for treating a condition associated with death of cells of nervous system comprising administering a therapeutically effective amount of a DR6 antagonist in combination with a p75 antagonist.

In some particular embodiments the condition associated with death of nervous system cells can be ALS (Lou Gehrig's disease) or SMA. Another embodiment provides methods for treating a disease of neuronal degeneration comprising administering a therapeutically effective amount of a DR6 antagonist.

### DR6 Antagonist Polypeptides

DR6 antagonists include those polypeptides which block, inhibit or interfere with the biological function of naturally occurring DR6. Specifically, soluble DR6 polypeptides include fragments, variants, or derivative thereof of a soluble DR6 polypeptide. Table 2 above describes the various domains of a human DR6 polypeptide. Similar domain structures can be deduced for DR6 polypeptides of other species, e.g., mouse or rat DR6. Soluble DR6 polypeptides typically lack the transmembrane domain of the DR6 polypeptide, and optionally lack the cytoplasmic domain of the DR6 polypeptide. For example, certain soluble human DR6 polypeptides lack amino acids 351-367 of SEQ ID NO:2, which comprises the transmembrane domain of human DR6. Another soluble human DR6 polypeptide lacks both the transmembrane domain and the intracellular domain (amino acids 350-655 of SEQ ID NO:2). Additionally, certain soluble DR6 polypeptides comprise one or more of the TNFR-like cysteine rich motifs and/or the entire extracellular domain (corresponding to amino acids 40 to 349 of SEQ ID NO:2, 40 to 350 of SEQ ID NO:2, 41 to 349 of SEQ ID NO:2 or 41 to 350 of SEQ ID NO:2) of the DR6 polypeptide. As one of skill in the art would appreciate, the entire extracellular domain of DR6 can comprise additional or fewer amino acids on either the C-terminal or N-terminal end of the extracellular domain polypeptide. The soluble antagonist DR6 polypeptide can or can not include the signal sequence.

In one embodiment, a soluble DR6 polypeptide comprises a modified Fc region.

A variant DR6 polypeptide can also vary in sequence from the corresponding wild-type polypeptide. In particular, certain amino acid substitutions can be introduced into the DR6 sequence without appreciable loss of a DR6 biological activity. In exemplary embodiments, a variant DR6 polypeptide contains one or more amino acid substitutions, and/or comprises an amino acid sequence which is at least 70%, 80%, 85%, 90%, 95%, 98% or 99% identical to a reference amino acid sequence selected from the group consisting of: amino acids 41 to 349 of SEQ ID NO:2 or equivalent fragments of SEQ ID NO:4 or 170. A variant DR6 polypeptide differing in sequence from any given fragment of SEQ ID NO:2, 4, or 170 can include one or more amino acid substitutions (conservative or non-conservative), one or more deletions, and/or one or more insertions. The soluble DR6 polypeptide can promote survival of cells of the neuronal system such as neurons and OPCs, e.g., in a mammal.

Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The non-polar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution.

Non-conservative substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, Ile, Phe or Trp) is substituted for, or by, one having a smaller side chain (e.g., Ala, Ser) or no side chain (e.g., Gly).

As would be well understood by a person of ordinary skill in the art, any of the fragments listed above can further include a secretory signal peptide at the N-terminus, e.g., amino acids 1 to 40 of SEQ ID NO:2 or amino acids 1 to 41 of SEQ ID NO:2. Soluble DR6 polypeptides can be cyclic. Cyclization of the soluble DR6 polypeptides reduces the conformational freedom of linear peptides and results in a more structurally constrained molecule. Many methods of peptide cyclization are known in the art. For example, “backbone to backbone” cyclization by the formation of an amide bond between the N-terminal and the C-terminal amino acid residues of the peptide. The “backbone to backbone” cyclization method includes the formation of disulfide bridges between two co-thio amino acid residues (e.g. cysteine, homocysteine). Certain soluble DR6 peptides described herein include modifications on the N- and C-terminus of the peptide to form a cyclic DR6 polypeptide. Such modifications include, but are not limited, to cysteine residues, acetylated cysteine residues, cysteine residues with a NH2 moiety and biotin. Other methods of peptide cyclization are described in Li & Roller. Curr. Top. Med. Chem. 3:325-341 (2002), which is incorporated by reference herein in its entirety.

In some embodiments, the DR6 antagonist polypeptide inhibits the formation of a complex between DR6 and p75. In some embodiments, the DR6 antagonist polypeptide inhibits the binding of DR6 to p75. In some embodiments, the DR6 antagonist polypeptide inhibis binding of DR6 to p75, but does not prevent DR6 binding to APP.

### Identifying New DR6 Antagonist Compounds

DR6 antagonists include any proteinaceous, chemical or synthetic compound which inhibits or decreases the activity of DR6 compared to the activity of DR6 in the absence of the antagonist compound. The DR6 antagonist compound can be one that inhibits binding of DR6 to p75. The DR6 antagonist compound can also be one that inhibits binding of DR6 to p75 but does not prevent binding of DR6 to APP.

One of ordinary skill in the art would know how to screen and test for DR6 antagonist compounds which would be useful in the methods described herein, for example by screening for compounds that modify nervous system cell survival using assays described elsewhere herein or known in the art.

### DR6 Antibodies or Antigen-Binding Fragments Thereof

DR6 antagonists also include DR6-antigen binding molecules, DR6-specific antibodies or antigen-binding fragments, variants, or derivatives which are antagonists of DR6 activity. For example, binding of certain DR6 antigen binding molecules or DR6 antibodies to DR6, as expressed in neurons inhibit apoptosis or promote cell survival.

In certain embodiments, the antibody is an antibody or antigen-binding fragment, variant or derivative of that specifically binds to DR6, wherein the antibody promotes survival of cells of the nervous system. In certain embodiments, the antibody is an antibody or antigen-binding fragment, variant or derivative of that specifically binds to DR6, wherein the antibody promotes proliferation, differentiation or survival of oligodendrocytes. In certain embodiments, the DR6 antibody is an antibody or antigen-binding fragment, variant or derivative thereof that specifically binds to DR6, wherein the antibody promotes myelination. In other embodiments, the DR6 antibody is an antibody or antigen-binding fragment, variant or derivative thereof that inhibits the formation of a complex between DR6 and p75. In other embodiments, the DR6 antibody is an antibody or antigen-binding fragment, variant or derivative thereof that inhibits binding of DR6 to p75. In other embodiments, the DR6 antibody is an antibody or antigen-binding fragment, variant or derivative thereof that inhibits binding of DR6 to p75 but does not prevent binding of DR6 to APP.

Certain DR6 antagonist antibodies specifically or preferentially bind to a particular DR6 polypeptide fragment or domain, for example, a DR6 polypeptide, fragment, variant, or derivative as described herein. Such DR6 polypeptide fragments include, but are not limited to, a DR6 polypeptide comprising, consisting essentially of, or consisting of one or more TNFR-like cysteine-rich motifs of DR6. Such fragments include for example, fragments comprising, consisting essentially of or consisting of amino acids 65 to 105 of SEQ ID NO:2; 106 to 145 of SEQ ID NO:2; 146 to 185 of SEQ ID NO:2; 186 to 212 of SEQ ID NO:2; 65 to 145 of SEQ ID NO:2; 65 to 185 of SEQ ID NO:2; 65 to 212 of SEQ ID NO:2; 106 to 185 of SEQ ID NO:2; 106 to 212 of SEQ ID NO:2; and 146 to 212 of SEQ ID NO:2. Such fragments also include amino acids 134-189 of SEQ ID NO:2; 168-189 of SEQ ID NO:2; and 134-168 of SEQ ID NO:2. Corresponding fragments of a variant DR6 polypeptide at least 70%, 75%, 80%, 85%, 90% or 95% identical to amino acids 65 to 105 of SEQ ID NO:2; 106 to 145 of SEQ ID NO:2; 146 to 185 of SEQ ID NO:2; 186 to 212 of SEQ ID NO:2; 65 to 145 of SEQ ID NO:2; 65 to 185 of SEQ ID NO:2; 65 to 212 of SEQ ID NO:2; 106 to 185 of SEQ ID NO:2; 106 to 212 of SEQ ID NO:2; 146 to 212 of SEQ ID NO:2; 134-189 of SEQ ID NO:2; 168-189 of SEQ ID NO:2; and 134-168 of SEQ ID NO:2 are also contemplated. In some embodiments, the DR6 antibody, antigen-binding fragment, variant, or derivative thereof requires both the Cys3 and Cys4 regions of DR6 to interact with DR6.

In other embodiments, the antibody is an antibody, or antigen-binding fragment, variant, or derivative thereof which specifically or preferentially binds to at least one epitope of DR6, where the epitope comprises, consists essentially of, or consists of at least about four to five amino acids of SEQ ID NO:2, 4, or 170, at least seven, at least nine, or between at least about 15 to about 30 amino acids of SEQ ID NO:2, 4, or 170. The amino acids of a given epitope of SEQ ID NO:2, 4, or 170 as described can be, but need not be contiguous or linear. In certain embodiments, the at least one epitope of DR6 comprises, consists essentially of, or consists of a non-linear epitope formed by the extracellular domain of DR6 as expressed on the surface of a cell or as a soluble fragment, e.g., fused to an IgG Fc region. Thus, in certain embodiments the at least one epitope of DR6 comprises, consists essentially of, or consists of at least 4, at least 5, at least 6, at least 7, at least 8, at least 9, at least 10, at least 15, at least 20, at least 25, between about 15 to about 30, or at least 10, 15, 20, 25, 30, 35, 40, 45, 50, 55, 60, 65, 70, 75, 80, 85, 90, 95, or 100 contiguous or non-contiguous amino acids of SEQ ID NO:2, 4, or 170 where non-contiguous amino acids form an epitope through protein folding.

In other embodiments, the antibody is an antibody, or antigen-binding fragment, variant, or derivative thereof which specifically or preferentially binds to at least one epitope of DR6, where the epitope comprises, consists essentially of, or consists of, in addition to one, two, three, four, five, six or more contiguous or non-contiguous amino acids of SEQ ID NO:2, 4, or 170 as described above, and an additional moiety which modifies the protein, e.g., a carbohydrate moiety can be included such that the DR6 antibody binds with higher affinity to modified target protein than it does to an unmodified version of the protein. Alternatively, the DR6 antibody does not bind the unmodified version of the target protein at all.

In certain aspects, the antibody is an antibody, or antigen-binding fragment, variant, or derivative thereof which specifically binds to a DR6 polypeptide or fragment thereof, or a DR6 variant polypeptide, with an affinity characterized by a dissociation constant (K_{D}) which is less than the K_{D} for a given reference monoclonal antibody.

In certain embodiments, an antibody, or antigen-binding fragment, variant, or derivative thereof binds specifically to at least one epitope of DR6 or fragment or variant described above, i.e., binds to such an epitope more readily than it would bind to an unrelated, or random epitope; binds preferentially to at least one epitope of DR6 or fragment or variant described above, i.e., binds to such an epitope more readily than it would bind to a related, similar, homologous, or analogous epitope; competitively inhibits binding of a reference antibody which itself binds specifically or preferentially to a certain epitope of DR6 or fragment or variant described above; or binds to at least one epitope of DR6 or fragment or variant described above with an affinity characterized by a dissociation constant K_{D} of less than about 5×10⁻² M, about 10⁻² M, about 5×10⁻³ M, about 10⁻³ M, about 5×10⁻⁴ M, about 10⁻⁴ M, about 5×10⁻⁵ M, about 10⁻⁵ M, about 5×10⁻⁶ M, about 10⁻⁶ M, about 5×10⁻⁷ M, about 10⁻⁷ M, about 5×10⁻⁸ M, about 10⁻⁸ M, about 5×10⁻⁹ M, about 10⁻⁹ M, about 5×10⁻¹⁰ M, about 10⁻¹⁰ M, about 5×10⁻¹¹ M, about 10⁻¹¹ M, about 5×10⁻¹² M, about 10⁻¹² M, about 5×10⁻¹³ M, about 10⁻¹³ M, about 5×10⁻¹⁴ M, about 10⁻¹⁴ M, about 5×10⁻¹⁵ M, or about 10⁻¹⁵ M. In a particular aspect, the antibody or fragment thereof preferentially binds to a human DR6 polypeptide or fragment thereof, relative to a murine DR6 polypeptide or fragment thereof. In another particular aspect, the antibody or fragment thereof preferentially binds to one or more DR6 polypeptides or fragments thereof, e.g., one or more mammalian DR6 polypeptides.

As used in the context of antibody binding dissociation constants, the term “about” allows for the degree of variation inherent in the methods utilized for measuring antibody affinity. For example, depending on the level of precision of the instrumentation used, standard error based on the number of samples measured, and rounding error, the term “about 10⁻² M” might include, for example, from 0.05 M to 0.005 M.

In specific embodiments, an antibody, or antigen-binding fragment, variant, or derivative thereof binds DR6 polypeptides or fragments or variants thereof with an off rate (k(off)) of less than or equal to 5×10⁻² sec⁻¹, 10.2 sec⁻¹, 5×10⁻³ sec⁻¹ or 10⁻³ sec⁻¹. Alternatively, an antibody, or antigen-binding fragment, variant, or derivative thereof binds DR6 polypeptides or fragments or variants thereof with an off rate (k(off)) of less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

In other embodiments, an antibody, or antigen-binding fragment, variant, or derivative thereof binds DR6 polypeptides or fragments or variants thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. Alternatively, an antibody, or antigen-binding fragment, variant, or derivative thereof binds DR6 polypeptides or fragments or variants thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

As used herein, the term “antigen binding domain” includes a site that specifically binds an epitope on an antigen (e.g., an epitope of DR6). The antigen binding domain of an antibody typically includes at least a portion of an immunoglobulin heavy chain variable region and at least a portion of an immunoglobulin light chain variable region. The binding site formed by these variable regions determines the specificity of the antibody.

In one embodiment, the DR6 antibody includes DR6 antibodies, or antigen-binding fragments, variants, or derivatives thereof which at least the antigen-binding domains of certain monoclonal antibodies, and fragments, variants, and derivatives thereof shown in Tables 3 and 4. Table 3 lists human anti-DR6 Fab regions identified from a phage display library. Table 4 lists mouse anti-DR6 antibodies derived from

TABLE 3
DR6-Specific Human Fabs

| | |
|---|---|
| 1 | M50-H01 |
| 2 | M51-H09 |
| 3 | M53-E04 |
| 4 | M53-F04 |
| 5 | M62-B02 |
| 6 | M63-E10 |
| 7 | M66-B03 |
| 8 | M67-G02 |
| 9 | M72-F03 |
| 10 | M73-C04 |

TABLE 4
DR6-Specific Murine Monoclonal Antibodies

| | |
|---|---|
| 1 | 1P1D6.3 |
| 2 | 1P2F2.1 |
| 3 | 1P5D10.2 |

In some embodiments, the DR6 antibody is a DR6 antibody, or antigen-binding fragment, variant or derivatives thereof, where the DR6 antibody specifically binds to the same DR6 epitope as a reference monoclonal Fab antibody fragment selected from the group consisting of M50-H01, M51-H09, M53-E04, M53-F04, M62-B02, M63-E10, M66-B03, M67-G02, M72-F03, and M73-C04 or a reference monoclonal antibody selected from the group consisting of 1P1D6.3, 1P2F2.1, and 1P5D10.2 (“5D10”) or 1P5D10.2(Y93A) (“5D10Y93A” or “Y93A”).

In some embodiments, the DR6 antibody is a DR6 antibody, or antigen-binding fragment, variant or derivatives thereof, where the DR6 antibody competitively inhibits a reference monoclonal Fab antibody fragment selected from the group consisting of M50-H01, M51-H09, M53-E04, M53-F04, M62-B02, M63-E10, M66-B03, M67-G02, M72-F03, and M73-C04 or a reference monoclonal antibody selected from the group consisting of 1P1D6.3, 1P2F2.1, 1P5D10.2 and 5D10Y93A from binding to DR6.

In some embodiments, the DR6 antibody is a DR6 antibody, or antigen-binding fragment, variant or derivatives thereof, where the DR6 antibody comprises an antigen binding domain identical to that of a monoclonal Fab antibody fragment selected from the group consisting of M50-H01, M51-H09, M53-E04, M53-F04, M62-B02, M63-E10, M66-B03, M67-G02, M72-F03, and M73-C04 or a reference monoclonal antibody selected from the group consisting of 1P1D6.3, 1P2F2.1, 1P5D10.2, and 5D10Y93A.

In some embodiments, the DR6 antibody is not an antibody selected from the group consisting of 3F4.48, 4B6.9.7 or 1E5.57 as described in International Publication No. WO2008/080045, filed Dec. 21, 2007. In some embodiments, the DR6 antibody is not antibody selected from the group consisting of antibodies that competitively inhibit binding of 3F4.48, 4B6.9.7 or 1E5.57 to DR6.

Methods of making antibodies are well known in the art and described herein. Once antibodies to various fragments of, or to the full-length DR6 without the signal sequence, have been produced, determining which amino acids, or epitope, of DR6 to which the antibody or antigen binding fragment binds can be determined by epitope mapping protocols as described herein as well as methods known in the art (e.g. double antibody-sandwich ELISA as described in “Chapter 11—Immunology,” Current Protocols in Molecular Biology, Ed. Ausubel et al., v. 2, John Wiley & Sons, Inc. (1996)). Additional epitope mapping protocols can be found in Morris, G. Epitope Mapping Protocols, New Jersey: Humana Press (1996), which are both incorporated herein by reference in their entireties. Epitope mapping can also be performed by commercially available means (i.e. ProtoPROBE, Inc. (Milwaukee, Wis.)).

Pair-wise binding experiments test the ability of two MAbs to bind simultaneously to the same antigen. MAbs directed against separate epitopes will bind independently, whereas MAbs directed against identical or closely related epitopes will interfere with each other's binding. These binding experiments with BIAcore are straightforward to carry out.

For example, one can use a capture molecule to bind the first Mab, followed by addition of antigen and second MAb sequentially. The sensorgrams will reveal: 1. how much of the antigen binds to first Mab, 2. to what extent the second MAb binds to the surface-attached antigen, 3. if the second MAb does not bind, whether reversing the order of the pair-wise test alters the results.

Peptide inhibition is another technique used for epitope mapping. This method can complement pair-wise antibody binding studies, and can relate functional epitopes to structural features when the primary sequence of the antigen is known. Peptides or antigen fragments are tested for inhibition of binding of different MAbs to immobilized antigen. Peptides which interfere with binding of a given MAb are assumed to be structurally related to the epitope defined by that MAb.

Additionally, antibodies produced which bind to any portion of DR6 can then be screened for their ability to act as an antagonist of DR6 for example, promoting survival of cells of the nervous system, treating a condition associated with death of cells of the nervous and preventing apoptosis of cells of the nervous system Antibodies can be screened for these and other properties according to methods described in detail in the Examples. Other functions of antibodies described herein can be tested using other assays as described in the Examples herein.

In one embodiment, a DR6 antagonist for use in the methods described herein is an antibody molecule, or antigen-binding fragment thereof. Unless it is specifically noted, as used herein a “fragment thereof” in reference to an antibody refers to an antigen-binding fragment, i.e., an antigen-specific fragment.

In one embodiment, a binding molecule or antigen binding molecule for use in the methods described herein comprises a synthetic constant region wherein one or more domains are partially or entirely deleted (“domain-deleted antibodies”). Certain methods described herein comprise administration of a DR6 antagonist antibody, or antigen-binding fragment thereof, in which at least a fraction of one or more of the constant region domains has been deleted or otherwise altered so as to provide desired biochemical characteristics such as reduced effector functions, the ability to non-covalently dimerize, increased ability to localize at the site of action, reduced serum half-life, or increased serum half-life when compared with a whole, unaltered antibody of approximately the same immunogenicity. For example, certain antibodies for use in the treatment methods described herein are domain deleted antibodies which comprise a polypeptide chain similar to an immunoglobulin heavy chain, but which lack at least a portion of one or more heavy chain domains.

In certain embodiments compatible modified antibodies will comprise domain deleted constructs or variants wherein the entire C_{H}2 domain has been removed (ΔC_{H}2 constructs). For other embodiments a short connecting peptide can be substituted for the deleted domain to provide flexibility and freedom of movement for the variable region. Those skilled in the art will appreciate that such constructs can be desirable under certain circumstances due to the regulatory properties of the C_{H}2 domain on the catabolic rate of the antibody. Domain deleted constructs can be derived using a vector (e.g., from Biogen IDEC Incorporated) encoding an IgG₁ human constant domain (see, e.g., WO 02/060955A2 and WO02/096948A2). This exemplary vector was engineered to delete the C_{H}2 domain and provide a synthetic vector expressing a domain deleted IgG₁ constant region.

In certain embodiments, modified antibodies are minibodies. Minibodies can be made using methods described in the art (see, e.g., see e.g., U.S. Pat. No. 5,837,821 or WO 94/09817A1).

In one embodiment, a DR6 antagonist antibody or fragment thereof comprises an immunoglobulin heavy chain having deletion or substitution of a few or even a single amino acid as long as it permits association between the monomeric subunits. For example, the mutation of a single amino acid in selected areas of the C_{H}2 domain can be enough to substantially reduce Fc binding and thereby increase localization to the intended site of action. Similarly, it can be desirable to simply delete that part of one or more constant region domains that control the effector function (e.g. complement binding) to be modulated. Such partial deletions of the constant regions can improve selected characteristics of the antibody (serum half-life) while leaving other desirable functions associated with the subject constant region domain intact. Moreover, as alluded to above, the constant regions of the disclosed antibodies can be synthetic through the mutation or substitution of one or more amino acids that enhances the profile of the resulting construct. In this respect it can be possible to disrupt the activity provided by a conserved binding site (e.g. Fc binding) while substantially maintaining the configuration and immunogenic profile of the modified antibody. Yet other embodiments comprise the addition of one or more amino acids to the constant region to enhance desirable characteristics such as effector function or provide for more cytotoxin or carbohydrate attachment. In such embodiments it can be desirable to insert or replicate specific sequences derived from selected constant region domains.

In certain DR6 antagonist antibodies or antigen-binding fragments thereof, the Fc portion can be mutated to decrease effector function using techniques known in the art. For example, modifications of the constant region can be used to modify disulfide linkages or oligosaccharide moieties that allow for enhanced localization due to increased antigen specificity or antibody flexibility. The resulting physiological profile, bioavailability and other biochemical effects of the modifications can easily be measured and quantified using well know immunological techniques without undue experimentation.

Antibodies that comprise, consist essentially of, or consist of, variants (including derivatives) of antibody molecules (e.g., the V_{H} regions and/or V_{L} regions) described herein, which antibodies or antigen-binding fragments thereof immunospecifically bind to a DR6 polypeptide. Standard techniques known to those of skill in the art can be used to introduce mutations in the nucleotide sequence encoding a binding molecule, including, but not limited to, site-directed mutagenesis and PCR-mediated mutagenesis which result in amino acid substitutions. In various embodiments, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference V_{H} region, V_{H}CDR1, V_{H}CDR2, V_{H}CDR3, V_{L} region, V_{L}CDR1, V₁CDR2, or V_{L}CDR3. A “conservative amino acid substitution” is one in which the amino acid residue is replaced with an amino acid residue having a side chain with a similar charge. Families of amino acid residues having side chains with similar charges have been defined in the art. Thus, a nonessential amino acid residue in an immunoglobulin polypeptide can be replaced with another amino acid residue from the same side chain family. In another embodiment, a string of amino acids can be replaced with a structurally similar string that differs in order and/or composition of side chain family members. Alternatively, mutations can be introduced randomly along all or part of the coding sequence, such as by saturation mutagenesis, and the resultant mutants can be screened for biological activity to identify mutants that retain activity.

For example, it is possible to introduce mutations only in framework regions or only in CDR regions of an antibody molecule. Introduced mutations can be silent or neutral missense mutations, i.e., have no, or little, effect on an antibody's ability to bind antigen. These types of mutations can be useful to optimize codon usage, or improve a hybridoma's antibody production. Alternatively, non-neutral missense mutations can alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein can routinely be expressed and the functional and/or biological activity of the encoded protein can be determined using techniques described herein or by routinely modifying techniques known in the art.

Modified forms of antibodies or antigen-binding fragments thereof can be made from whole precursor or parent antibodies using techniques known in the art. Exemplary techniques are discussed in more detail herein.

DR6 antagonist antibodies or antigen-binding fragments thereof can be made or manufactured using techniques that are known in the art. In certain embodiments, antibody molecules or fragments thereof are “recombinantly produced,” i.e., are produced using recombinant DNA technology. Exemplary techniques for making antibody molecules or fragments thereof are discussed in more detail elsewhere herein. DR6 antagonist antibodies or fragments thereof can be generated by any suitable method known in the art.

In certain embodiments, a DR6 antagonist antibody or antigen-binding fragment thereof will not elicit a deleterious immune response in the animal to be treated, e.g., in a human. In one embodiment, DR6 antagonist antibodies or antigen-binding fragments thereof are modified to reduce their immunogenicity using art-recognized techniques. For example, antibodies can be humanized, primatized, deimmunized, or chimeric antibodies can be made. These types of antibodies are derived from a non-human antibody, typically a murine or primate antibody, that retains or substantially retains the antigen-binding properties of the parent antibody, but which is less immunogenic in humans. This can be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81:6851-6855 (1984); Morrison et al., Adv. Immunol. 44:65-92 (1988); Verhoeyen et al., Science 239:1534-1536 (1988); Padlan, Molec. Immun. 28:489-498 (1991); Padlan, Molec. Immun. 31:169-217 (1994), and U.S. Pat. Nos. 5,585,089, 5,693,761, 5,693,762, and 6,190,370, all of which are hereby incorporated by reference in their entirety.

Recombinant expression of an antibody, or fragment, derivative or analog thereof, e.g., a heavy or light chain of an antibody which is a DR6 antagonist, requires construction of an expression vector containing a polynucleotide that encodes the antibody. Once a polynucleotide encoding an antibody molecule or a heavy or light chain of an antibody, or portion thereof (e.g., containing the heavy or light chain variable domain), has been obtained, the vector for the production of the antibody molecule can be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. Also considered herein are replicable vectors comprising a nucleotide sequence encoding an antibody molecule, or a heavy or light chain thereof, or a heavy or light chain variable domain, operably linked to a promoter. Such vectors can include the nucleotide sequence encoding the constant region of the antibody molecule (see, e.g., PCT Publication WO 86/05807; PCT Publication WO 89/01036; and U.S. Pat. No. 5,122,464) and the variable domain of the antibody can be cloned into such a vector for expression of the entire heavy or light chain.

The expression vector is transferred to a host cell by conventional techniques and the transfected cells are then cultured by conventional techniques to produce an antibody. Thus, host cells containing a polynucleotide encoding an antibody, or a heavy or light chain thereof, operably linked to a heterologous promoter are also described herein. In certain embodiments for the expression of double-chained antibodies, vectors encoding both the heavy and light chains can be co-expressed in the host cell for expression of the entire immunoglobulin molecule.

A variety of host-expression vector systems can be utilized to express antibody molecules. A host cell can be co-transfected with two expression vectors, the first vector encoding a heavy chain derived polypeptide and the second vector encoding a light chain derived polypeptide. The two vectors can contain identical selectable markers which enable equal expression of heavy and light chain polypeptides. Alternatively, a single vector can be used which encodes both heavy and light chain polypeptides. In such situations, the light chain is advantageously placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains can comprise cDNA or genomic DNA.

Once an antibody molecule has been recombinantly expressed, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Alternatively, a method for increasing the affinity of antibodies is disclosed in US 2002 0123057 A1.

Furthermore, as described in more detail below, any of the DR6 antibodies or antibody fragments as described herein can be conjugated (covalently linked) to one or more polymers. In one particular embodiment, an antibody fragment that recognizes a specific epitope, for example, a Fab, F(ab′)₂, Fv fragment or single chain antibody can be conjugated to a polymer. Examples of polymers suitable for such conjugation include polypeptides, sugar polymers and polyalkylene glycol chains (as described in more detail below). The class of polymer generally used is a polyalkylene glycol. Polyethylene glycol (PEG) is most frequently used. PEG moieties, e.g., 1, 2, 3, 4 or 5 PEG polymers, can be conjugated to DR6 antibodies or fragments thereof to increase serum half life. PEG moieties are non-antigenic and essentially biologically inert. PEG moieties used can be branched or unbranched.

### Polynucleotides Encoding DR6 Antibodies

The polynucleotides described herein include nucleic acid molecules encoding DR6 antibodies, or antigen-binding fragments, variants, or derivatives thereof.

In one embodiment, the polynucleotide an isolated polynucleotide comprising, consisting essentially of, or consisting of a nucleic acid encoding an immunoglobulin heavy chain variable region (VH), where at least one of the CDRs of the heavy chain variable region or at least two of the VH-CDRs of the heavy chain variable region are at least 80%, 85%, 90% or 95% identical to reference heavy chain VH-CDR1, VH-CDR2, or VH-CDR3 amino acid sequences from monoclonal DR6 antibodies disclosed herein. Alternatively, the VH-CDR1, VH-CDR2, and VH-CDR3 regions of the VH are at least 80%, 85%, 90% or 95% identical to reference heavy chain VH-CDR1, VH-CDR2, and VH-CDR3 amino acid sequences from monoclonal DR6 antibodies disclosed herein. Thus, according to this embodiment a heavy chain variable region has VH-CDR1, VH-CDR2, or VH-CDR3 polypeptide sequences related to the polypeptide sequences shown in Table 5.

In another embodiment, the polynucleotide is an isolated polynucleotide comprising, consisting essentially of, or consisting of a nucleic acid encoding an immunoglobulin light chain variable region (VL), where at least one of the VL-CDRs of the light chain variable region or at least two of the VL-CDRs of the light chain variable region are at least 80%, 85%, 90% or 95% identical to reference light chain VL-CDR1, VL-CDR2, or VL-CDR3 amino acid sequences from monoclonal DR6 antibodies disclosed herein. Alternatively, the VL-CDR1, VL-CDR2, and VL-CDR3 regions of the VL are at least 80%, 85%, 90% or 95% identical to reference light chain VL-CDR1, VL-CDR2, and VL-CDR3 amino acid sequences from monoclonal DR6 antibodies disclosed herein. Thus, according to this embodiment a light chain variable region has VL-CDR1, VL-CDR2, or VL-CDR3 polypeptide sequences related to the polypeptide sequences shown in Table 5.

TABLE 5
DR6 Antibody Sequence SEQ ID NOs

| | VH | VH | VH | VH | VH | | | VL | VL | VL |
|---|---|---|---|---|---|---|---|---|---|---|
| Antibody | PN | PP | CDR1 | CDR2 | CDR3 | VL PN | VL PP | CDR1 | CDR2 | CDR3 |
| M50-H01 | 6 | 7 | 8 | 9 | 10 | 11 | 12 | 13 | 14 | 15 |
| M51-H09 | 16 | 17 | 18 | 19 | 20 | 21 | 22 | 23 | 24 | 25 |
| M53-E04 | 26 | 27 | 28 | 29 | 30 | 31 | 32 | 33 | 34 | 35 |
| M53-F04 | 36 | 37 | 38 | 39 | 40 | 41 | 42 | 43 | 44 | 45 |
| M62-B02 | 46 | 47 | 48 | 49 | 50 | 51 | 52 | 53 | 54 | 55 |
| M63-E10 | 56 | 57 | 58 | 59 | 60 | 61 | 62 | 63 | 64 | 65 |
| M66-B03 | 66 | 67 | 68 | 69 | 70 | 71 | 72 | 73 | 74 | 75 |
| M67-G02 | 76 | 77 | 78 | 79 | 80 | 81 | 82 | 83 | 84 | 85 |
| M72-F03 | 86 | 87 | 88 | 89 | 90 | 91 | 92 | 93 | 94 | 95 |
| M73-C04 | 96 | 97 | 98 | 99 | 100 | 101 | 102 | 103 | 104 | 105 |
| 1P1D6.3 | 106 | 107 | 108 | 109 | 110 | 111 | 112 | 113 | 114 | 115 |
| 1P2F2.1 | 116 | 117 | 118 | 119 | 120 | 121 | 122 | 123 | 124 | 125 |
| 1P5D10.2 | 126 | 127 | 128 | 129 | 130 | 131 | 132 | 133 | 134 | 135 |
| 1P5D10.2 | 126 | 127 | 128 | 129 | 130 | * | 167 | 133 | 134 | 168 |
| Y93A | | | | | | | | | | |

** a nucleic acid comprising the sequence of SEQ ID NO: 131 except wherein nucleotides 277-279 are GCT, GCC, GCA, or GCG.

In certain embodiments, an antibody or antigen-binding fragment comprising the VH encoded by the polynucleotide specifically or preferentially binds to DR6. In certain embodiments the nucleotide sequence encoding the VH polypeptide is altered without altering the amino acid sequence encoded thereby. For instance, the sequence can be altered for improved codon usage in a given species, to remove splice sites, or the remove restriction enzyme sites. Sequence optimizations such as these are described in the examples and are well known and routinely carried out by those of ordinary skill in the art.

In another embodiment, the polynucleotide is isolated polynucleotide comprising, consisting essentially of, or consisting of a nucleic acid encoding an immunoglobulin heavy chain variable region (VH) in which the VH-CDR1, VH-CDR2, and VH-CDR3 regions have polypeptide sequences which are identical to the VH-CDR1, VH-CDR2, and VH-CDR3 groups shown in Table 5. In certain embodiments, an antibody or antigen-binding fragment comprising the VH encoded by the polynucleotide specifically or preferentially binds to DR6.

In some embodiments, the polynucleotide is an isolated polynucleotide comprising a nucleic acid which encodes an antibody VH polypeptide, where the VH polypeptide comprises VH-CDR1, VH-CDR2, and VH-CDR3 amino acid sequences selected from the group consisting of: SEQ ID NOs: 8, 9, and 10; SEQ ID NOs: 18, 19, and 20; SEQ ID NOs: 28, 29, and 30; SEQ ID NOs: 38, 39, and 40; SEQ ID NOs: 48, 49, and 50; SEQ ID NOs: 58, 59, and 60; SEQ ID NOs: 68, 69, and 70; SEQ ID NOs: 78, 79, and 80; SEQ ID NOs: 88, 89, and 90; SEQ ID NOs: 98, 99, and 100; SEQ ID NOs: 108, 109, and 110; SEQ ID NOs: 118, 119, and 120; and SEQ ID NOs: 128, 129, and 130; and where an antibody or antigen binding fragment thereof comprising the VH-CDR3 specifically binds to DR6.

In certain embodiments, an antibody or antigen-binding fragment comprising the VL encoded by the polynucleotide specifically or preferentially binds to DR6.

In another embodiment, the polynucleotide is an isolated polynucleotide comprising, consisting essentially of, or consisting of a nucleic acid encoding an immunoglobulin light chain variable region (VL) in which the VL-CDR1, VL-CDR2, and VL-CDR3 regions have polypeptide sequences which are identical to the VL-CDR1, VL-CDR2, and VL-CDR3 groups shown in Table 5. In certain embodiments, an antibody or antigen-binding fragment comprising the VL encoded by the polynucleotide specifically or preferentially binds to DR6.

In a further aspect, the polynucleotide is an isolated polynucleotide comprising, consisting essentially of, or consisting of a nucleic acid encoding an immunoglobulin light chain variable region (VL) in which the VL-CDR1, VL-CDR2. and VL-CDR3 regions are encoded by nucleotide sequences which are identical to the nucleotide sequences which encode the VL-CDR1, VL-CDR2, and VL-CDR3 groups shown in Table 5. In certain embodiments, an antibody or antigen-binding fragment comprising the VL encoded by the polynucleotide specifically or preferentially binds to DR6.

In some embodiments, the polynucleotide is an isolated polynucleotide comprising a nucleic acid which encodes an antibody VL polypeptide, wherein said VL polypeptide comprises VL-CDR1, VL-CDR2, and VL-CDR3 amino acid sequences selected from the group consisting of: SEQ ID NOs: 13, 14, and 15; SEQ ID NOs: 23, 24, and 25; SEQ ID NOs: 33, 34, and 35; SEQ ID NOs: 43, 44, and 45; SEQ ID NOs: 53, 54, and 55; SEQ ID NOs: 63, 64, and 65; SEQ ID NOs: 73, 74, and 75; SEQ ID NOs: 83, 84, and 85; SEQ ID NOs: 93, 94, and 95; SEQ ID NOs: 103, 104, and 105; SEQ ID NOs: 113, 114, and 115; SEQ ID NOs: 123, 124, and 125; SEQ ID NOs: 133, 134, and 135; and SEQ ID NOs: 133, 134, and 168; and wherein an antibody or antigen binding fragment thereof comprising said VL-CDR3 specifically binds to DR6.

In a further embodiment, the polynucleotide can be an isolated polynucleotide comprising, consisting essentially of, or consisting of a nucleic acid encoding a VH at least 80%, 85%, 90% 95% or 100% identical to a reference VH polypeptide sequence selected from the group consisting of SEQ ID NOs: 7, 17, 27, 37, 47, 57, 67, 77, 87, 97, 107, 117 and 127. In certain embodiments, an antibody or antigen-binding fragment comprising the VH encoded by the polynucleotide specifically or preferentially binds to DR6.

In another aspect, the polynucleotide can be an isolated polynucleotide comprising, consisting essentially of, or consisting of a nucleic acid sequence encoding a VH having a polypeptide sequence selected from the group consisting of SEQ ID NOs: 7, 17, 27, 37, 47, 57, 67, 77, 87, 97, 107, 117 and 127. In certain embodiments, an antibody or antigen-binding fragment comprising the VH encoded by the polynucleotide specifically or preferentially binds to DR6.

In a further embodiment, the polynucleotide can be an isolated polynucleotide comprising, consisting essentially of, or consisting of a VH-encoding nucleic acid at least 800%, 85%, 90% 95% or 100% identical to a reference nucleic acid sequence selected from the group consisting of SEQ ID NOs: 6, 16, 26, 36, 46, 56, 66, 76, 86, 96, 106, 116, and 126. In certain embodiments, an antibody or antigen-binding fragment comprising the VH encoded by such polynucleotides specifically or preferentially binds to DR6.

In another aspect, the polynucleotide can be an isolated polynucleotide comprising, consisting essentially of, or consisting of a nucleic acid sequence encoding a VH, where the amino acid sequence of the VH is selected from the group consisting of SEQ ID NOs: 7, 17, 27, 37, 47, 57, 67, 77, 87, 97, 107, 117 and 127. The polynucleotide can also be an isolated polynucleotide comprising, consisting essentially of, or consisting of a nucleic acid sequence encoding a VH, where the sequence of the nucleic acid is selected from the group consisting of SEQ ID NOs: 6, 16, 26, 36, 46, 56, 66, 76, 86, 96, 106, 116, and 126. In certain embodiments, an antibody or antigen-binding fragment comprising the VH encoded by such polynucleotides specifically or preferentially binds to DR6.

In a further embodiment, the polynucleotide can be an isolated polynucleotide comprising, consisting essentially of, or consisting of a nucleic acid encoding a VL at least 80%, 85%, 90% 95% or 100% identical to a reference VL polypeptide sequence having an amino acid sequence selected from the group consisting of SEQ ID NOs: 12, 22, 32, 42, 52, 62, 72, 82, 92, 102, 112, 122, 132, and 167. In a further embodiment, the polynucleotide can be an isolated polynucleotide comprising, consisting essentially of, or consisting of a VL-encoding nucleic acid at least 80%, 85%, 90% 95% or 100%/identical to a reference nucleic acid sequence selected from the group consisting of SEQ ID NOs: 11, 21, 31, 41, 51, 61, 71, 81, 91, 101, 111, 121, and 131 and a nucleic acid comprising the sequence of SEQ ID NO:131 except wherein nucleotides 277-279 are GCT, GCC, GCA, or GCG. In certain embodiments, an antibody or antigen-binding fragment comprising the VL encoded by such polynucleotides specifically or preferentially binds to DR6.

In another aspect, the polynucleotide can be an isolated polynucleotide comprising, consisting essentially of, or consisting of a nucleic acid sequence encoding a VL having a polypeptide sequence selected from the group consisting of SEQ ID NOs: 12, 22, 32, 42, 52, 62, 72, 82, 92, 102, 112, 122, 132, and 167. The polynucleotide can be an isolated polynucleotide comprising, consisting essentially of, or consisting of a nucleic acid sequence encoding a VL, where the sequence of the nucleic acid is selected from the group consisting of SEQ ID NOs: 11, 21, 31, 41, 51, 61, 71, 81, 91, 101, 111, 121, and 131 and a nucleic acid comprising the sequence of SEQ ID NO:131 except wherein nucleotides 277-279 are GCT, GCC, GCA, or GCG. In certain embodiments, an antibody or antigen-binding fragment comprising the VL encoded by such polynucleotides specifically or preferentially binds to DR6.

In certain embodiments, an antibody or antigen-binding fragment thereof comprising, consisting essentially of, or consisting of a VH and/or VL encoded by one or more of the polynucleotides described above specifically or preferentially binds to the same DR6 epitope as a reference monoclonal Fab antibody fragment selected from the group consisting of M50-H01, M51-H09, M53-E04, M53-F04, M62-B02, M63-E10, M66-B03, M67-G02, M72-F03, and M73-C04 or a reference monoclonal antibody selected from the group consisting of 1P1D6.3, 1P2F2.1, 1P5D10.2, and 5D10Y93A or will competitively inhibit such a monoclonal antibody or fragment from binding to DR6.

In certain embodiments, an antibody or antigen-binding fragment thereof comprising, consisting essentially of, or consisting of a VH and/or VL encoded by one or more of the polynucleotides described above specifically or preferentially binds to a DR6 polypeptide or fragment thereof, or a DR6 variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Any of the polynucleotides described above can further include additional nucleic acids, encoding, e.g., a signal peptide to direct secretion of the encoded polypeptide, antibody constant regions as described herein, or other heterologous polypeptides as described herein.

Also, as described in more detail elsewhere herein, the compositions include compositions comprising the polynucleotides comprising one or more of the polynucleotides described above. In one embodiment, the compositions includes compositions comprising a first polynucleotide and second polynucleotide wherein said first polynucleotide encodes a VH polypeptide as described herein and wherein said second polynucleotide encodes a VL polypeptide as described herein. Specifically a composition which comprises, consists essentially of, or consists of a VH polynucleotide, and a VL polynucleotide, wherein the VH polynucleotide and the VL polynucleotide encode polypeptides, respectively at least 80%, 85%, 90% 95% or 100% identical to reference VH and VL polypeptide amino acid sequences selected from the group consisting of SEQ ID NOs: 7 and 12, 17 and 22, 27 and 32, 37 and 42, 47 and 52, 57 and 62, 67 and 72, 77 and 82, 87 and 92, 97 and 102, 107 and 112, 117 and 122, 127 and 132, and 127 and 167. Or alternatively, a composition which comprises, consists essentially of, or consists of a VH polynucleotide, and a VL polynucleotide at least 80%, 85%, 90% 95% or 100% identical, respectively, to reference VL and VL nucleic acid sequences selected from the group consisting of SEQ ID NOs: 6 and 11, 16 and 21, 26 and 31, 36 and 41, 46 and 51, 56 and 61, 66 and 71, 76 and 81, 86 and 91, 96 and 101, 106 and 111, 116 and 121, and 126 and 131, and 126 and 131 wherein nucleotides 277-279 are GCT, GCC, GCA, or GCG. In certain embodiments, an antibody or antigen-binding fragment comprising the VH and VL encoded by the polynucleotides in such compositions specifically or preferentially binds to DR6.

The polynucleotides described herein also include fragments of the polynucleotides, as described elsewhere. Additionally polynucleotides which encode fusion polynucleotides, Fab fragments, and other derivatives, as described herein, are also contemplated.

The polynucleotides can be produced or manufactured by any method known in the art. For example, if the nucleotide sequence of the antibody is known, a polynucleotide encoding the antibody can be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding a DR6 antibody, or antigen-binding fragment, variant, or derivative thereof can be generated from nucleic acid from a suitable source. If a clone containing a nucleic acid encoding a particular antibody is not available, but the sequence of the antibody molecule is known, a nucleic acid encoding the antibody can be chemically synthesized or obtained from a suitable source (e.g., an antibody cDNA library, or a cDNA library generated from, or nucleic acid, such as poly A+RNA, isolated from, any tissue or cells expressing the antibody or other DR6 antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody or other DR6 antibody. Amplified nucleic acids generated by PCR can then be cloned into replicable cloning vectors using any method well known in the art.

Once the nucleotide sequence and corresponding amino acid sequence of the DR6 antibody, or antigen-binding fragment, variant, or derivative thereof is determined, its nucleotide sequence can be manipulated using methods well known in the art for the manipulation of nucleotide sequences, e.g., recombinant DNA techniques, site directed mutagenesis, PCR, etc. (see, for example, the techniques described in Sambrook et al., Molecular Cloning, A Laboratory Manual, 2d Ed., Cold Spring Harbor Laboratory, Cold Spring Harbor, N.Y. (1990) and Ausubel et al., eds., Current Protocols in Molecular Biology, John Wiley & Sons, NY (1998), which are both incorporated by reference herein in their entireties), to generate antibodies having a different amino acid sequence, for example to create amino acid substitutions, deletions, and/or insertions.

A polynucleotide encoding a DR6 antibody, or antigen-binding fragment, variant, or derivative thereof can be composed of any polyribonucleotide or polydeoxribonucleotide, which can be unmodified RNA or DNA or modified RNA or DNA. For example, a polynucleotide encoding DR6 antibody, or antigen-binding fragment, variant, or derivative thereof can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, a polynucleotide encoding a DR6 antibody, or antigen-binding fragment, variant, or derivative thereof can be composed of triple-stranded regions comprising RNA or DNA or both RNA and DNA. A polynucleotide encoding a DR6 antibody, or antigen-binding fragment, variant, or derivative thereof can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

An isolated polynucleotide encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations can be introduced by standard techniques, such as site-directed mutagenesis and PCR-mediated mutagenesis. Conservative amino acid substitutions can be made at one or more non-essential amino acid residues.

### DR6 Antibody Polypeptides

Isolated polypeptides which make up DR6 antibodies, and polynucleotides encoding such polypeptides are also described herein. DR6 antibodies comprise polypeptides, e.g., amino acid sequences encoding DR6-specific antigen binding regions derived from immunoglobulin molecules. A polypeptide or amino acid sequence “derived from” a designated protein refers to the origin of the polypeptide having a certain amino acid sequence. In certain cases, the polypeptide or amino acid sequence which is derived from a particular starting polypeptide or amino acid sequence has an amino acid sequence that is essentially identical to that of the starting sequence, or a portion thereof, wherein the portion consists of at least 10-20 amino acids, at least 20-30 amino acids, at least 30-50 amino acids, or which is otherwise identifiable to one of ordinary skill in the art as having its origin in the starting sequence.

In one embodiment, the polypeptide can be an isolated polypeptide comprising, consisting essentially of, or consisting of an immunoglobulin heavy chain variable region (VH), where at least one of VH-CDRs of the heavy chain variable region or at least two of the VH-CDRs of the heavy chain variable region are at least 80%, 85%, 90% or 95% identical to reference heavy chain VH-CDR1, VH-CDR2 or VH-CDR3 amino acid sequences from monoclonal DR6 antibodies disclosed herein. Alternatively, the VH-CDR1, VH-CDR2 and VH-CDR3 regions of the VH are at least 80%, 85%, 90% or 95% identical to reference heavy chain VH-CDR1, VH-CDR2 and VH-CDR3 amino acid sequences from monoclonal DR6 antibodies disclosed herein. Thus, according to this embodiment a heavy chain variable region has VH-CDR1, VH-CDR2 and VH-CDR3 polypeptide sequences related to the groups shown in Table 5, supra. While Table 5 shows VH-CDRs defined by the Kabat system, other CDR definitions, e.g., VH-CDRs defined by the Chothia system, are also described. In certain embodiments, an antibody or antigen-binding fragment comprising the VH specifically or preferentially binds to DR6.

In another embodiment, the polypeptide can be an isolated polypeptide comprising, consisting essentially of, or consisting of an immunoglobulin heavy chain variable region (VH) in which the VH-CDR1, VH-CDR2 and VH-CDR3 regions have polypeptide sequences which are identical to the VH-CDR1, VH-CDR2 and VH-CDR3 groups shown in Table 5. In certain embodiments, an antibody or antigen-binding fragment comprising the VH specifically or preferentially binds to DR6.

In another embodiment, the polypeptide can be an isolated polypeptide comprising, consisting essentially of, or consisting of an immunoglobulin heavy chain variable region (VH) in which the VH-CDR1, VH-CDR2 and VH-CDR3 regions have polypeptide sequences which are identical to the VH-CDR1, VH-CDR2 and VH-CDR3 groups shown in Table 5, except for one, two, three, four, five, or six amino acid substitutions in any one VH-CDR. In larger CDRs, e.g., VH-CDR-3, additional substitutions can be made in the CDR, as long as the a VH comprising the VH-CDR specifically or preferentially binds to DR6. In certain embodiments the amino acid substitutions are conservative. In certain embodiments, an antibody or antigen-binding fragment comprising the VH specifically or preferentially binds to DR6.

In some embodiments, the polypeptide can be an isolated polypeptide comprising, consisting essentially of, or consisting of an immunoglobulin heavy chain variable region (VH) in which the VH-CDR1, VH-CDR2 and VH-CDR3 regions have polypeptide sequences selected from the group consisting of: SEQ ID NOs: 8, 9, and 10; SEQ ID NOs: 18, 19, and 20; SEQ ID NOs: 28, 29, and 30; SEQ ID NOs: 38, 39, and 40; SEQ ID NOs: 48, 49, and 50; SEQ ID NOs: 58, 59, and 60; SEQ ID NOs: 68, 69, and 70; SEQ ID NOs: 78, 79, and 80; SEQ ID NOs: 88, 89, and 90; SEQ ID NOs: 98, 99, and 100; SEQ ID NOs: 108, 109, and 110; SEQ ID NOs: 118, 119, and 120; and SEQ ID NOs: 128, 129 and 130, except for one, two, three, four, five or six amino acid substitutions in at least one of said VH-CDRs.

In some embodiments, the polypeptide can be an isolated polypeptide comprising, consisting essentially of, or consisting of an immunoglobulin heavy chain variable region (VH) in which the VH-CDR1, VH-CDR2 and VH-CDR3 regions have polypeptide sequences selected from the group consisting of: SEQ ID NOs: 8, 9, and 10; SEQ ID NOs: 18, 19, and 20; SEQ ID NOs: 28, 29, and 30; SEQ ID NOs: 38, 39, and 40; SEQ ID NOs: 48, 49, and 50; SEQ ID NOs: 58, 59, and 60; SEQ ID NOs: 68, 69, and 70; SEQ ID NOs: 78, 79, and 80; SEQ ID NOs: 88, 89, and 90; SEQ ID NOs: 98, 99, and 100; SEQ ID NOs: 108, 109, and 110; SEQ ID NOs: 118, 119, and 120; and SEQ ID NOs: 128, 129 and 130.

In a further embodiment, the polypeptide can be an isolated polypeptide comprising, consisting essentially of, or consisting of a VH polypeptide at least 80%, 85%, 90% 95% or 100% identical to a reference VH polypeptide amino acid sequence selected from the group consisting of SEQ ID NOs: 7, 17, 27, 37, 47, 57, 67, 77, 87, 97, 107, 117, and 127. In certain embodiments, an antibody or antigen-binding fragment comprising the VH polypeptide specifically or preferentially binds to DR6.

In another aspect, the polypeptide can be an isolated polypeptide comprising, consisting essentially of, or consisting of a VH polypeptide selected from the group consisting of SEQ ID NOs: 7, 17, 27, 37, 47, 57, 67, 77, 87, 97, 107, 117, and 127. In certain embodiments, an antibody or antigen-binding fragment comprising the VH polypeptide specifically or preferentially binds to DR6.

In another embodiment, the polypeptide can be an isolated polypeptide comprising, consisting essentially of, or consisting of an immunoglobulin light chain variable region (VL), where at least one of the VL-CDRs of the light chain variable region or at least two of the VL-CDRs of the light chain variable region are at least 80%, 85%, 90% or 95% identical to reference light chain VL-CDR1, VL-CDR2 or VL-CDR3 amino acid sequences from monoclonal DR6 antibodies disclosed herein. Alternatively, the VL-CDR1, VL-CDR2 and VL-CDR3 regions of the VL are at least 80%, 85%, 90% or 95% identical to reference light chain VL-CDR1, VL-CDR2 and VL-CDR3 amino acid sequences from monoclonal DR6 antibodies disclosed herein. Thus, according to this embodiment a light chain variable region has VL-CDR1, VL-CDR2 and VL-CDR3 polypeptide sequences related to the polypeptides shown in Table 5. While Table 5 shows VL-CDRs defined by the Kabat system, other CDR definitions, e.g., VL-CDRs defined by the Chothia system, are also described. In certain embodiments, an antibody or antigen-binding fragment comprising the VL polypeptide specifically or preferentially binds to DR6.

In another embodiment, the polypeptide can be an isolated polypeptide comprising, consisting essentially of, or consisting of an immunoglobulin light chain variable region (VL) in which the VL-CDR1, VL-CDR2 and VL-CDR3 regions have polypeptide sequences which are identical to the VL-CDR1, VL-CDR2 and VL-CDR3 groups shown in Table 5. In certain embodiments, an antibody or antigen-binding fragment comprising the VL polypeptide specifically or preferentially binds to DR6.

In another embodiment, the polypeptide can be an isolated polypeptide comprising, consisting essentially of, or consisting of an immunoglobulin heavy chain variable region (VL) in which the VL-CDR1, VL-CDR2 and VL-CDR3 regions have polypeptide sequences which are identical to the VL-CDR1, VL-CDR2 and VL-CDR3 groups shown in Table 5, except for one, two, three, four, five, or six amino acid substitutions in any one VL-CDR. In larger CDRs, additional substitutions can be made in the VL-CDR, as long as the a VL comprising the VL-CDR specifically or preferentially binds to DR6. In certain embodiments the amino acid substitutions are conservative. In certain embodiments, an antibody or antigen-binding fragment comprising the VL specifically or preferentially binds to DR6.

In some embodiments, the polypeptide can be an isolated polypeptide comprising, consisting essentially of, or consisting of an immunoglobulin heavy chain variable region (VL) in which the VL-CDR1, VL-CDR2 and VL-CDR3 regions have polypeptide sequences selected from the group consisting of: SEQ ID NOs: 13, 14, and 15; SEQ ID NOs: 23, 24, and 25; SEQ ID NOs: 33, 34, and 35; SEQ ID NOs: 43, 44, and 45; SEQ ID NOs: 53, 54, and 55; SEQ ID NOs: 63, 64, and 65; SEQ ID NOs: 73, 74, and 75; SEQ ID NOs: 83, 84, and 85; SEQ ID NOs: 93, 94, and 95; SEQ ID NOs: 103, 104, and 105; SEQ ID NOs: 113, 114 and 115; SEQ ID NOs: 123, 124, and 125; SEQ ID NOs: 133, 134 and 135, and SEQ ID NOs: 133, 134 and 168, except for one, two, three, four, five or six amino acid substitutions in at least one of said VL-CDRs.

In some embodiments, the polypeptide can be an isolated polypeptide comprising, consisting essentially of, or consisting of an immunoglobulin heavy chain variable region (VL) in which the VL-CDR1, VL-CDR2 and VL-CDR3 regions have polypeptide sequences selected from the group consisting of: SEQ ID NOs: 13, 14, and 15; SEQ ID NOs: 23, 24, and 25; SEQ ID NOs: 33, 34, and 35; SEQ ID NOs: 43, 44, and 45; SEQ ID NOs: 53, 54, and 55; SEQ ID NOs: 63, 64, and 65; SEQ ID NOs: 73, 74, and 75; SEQ ID NOs: 83, 84, and 85; SEQ ID NOs: 93, 94, and 95; SEQ ID NOs: 103, 104, and 105; SEQ ID NOs: 113, 114 and 115; SEQ ID NOs: 123, 124, and 125; SEQ ID NOs: 133, 134 and 135, and SEQ ID NOs: 133, 134, and 168.

In a further embodiment, the polypeptide can be an isolated polypeptide comprising, consisting essentially of, or consisting of a VL. polypeptide at least 80%, 85%, 90% 95% or 100% identical to a reference VL polypeptide sequence selected from the group consisting of SEQ ID NOs: 12, 22, 32, 42, 52, 62, 72, 82, 92, 102, 112, 122, 132, and 167. In certain embodiments, an antibody or antigen-binding fragment comprising the VL polypeptide specifically or preferentially binds to DR6.

In another aspect, the polypeptide can be an isolated polypeptide comprising, consisting essentially of, or consisting of a VL polypeptide selected from the group consisting of SEQ ID NOs: 12, 22, 32, 42, 52, 62, 72, 82, 92, 102, 112, 122, 132, and 167. In certain embodiments, an antibody or antigen-binding fragment comprising the VL polypeptide specifically or preferentially binds to DR6.

In certain embodiments, an antibody or antigen-binding fragment thereof comprising, consisting essentially of, one or more of the VH and/or VL polypeptides described above specifically or preferentially binds to the same DR6 epitope as a reference monoclonal Fab antibody fragment selected from the group consisting of M50-H01, M51-H09, M53-E04, M53-F04, M62-B02, M63-E10, M66-B03, M67-G02, M72-F03, and M73-C04 or a reference monoclonal antibody selected from the group consisting of 1P1D6.3, 1P2F2.1, 1P5D10.2, and 5D10Y93A, or will competitively inhibit such a monoclonal antibody or fragment from binding to DR6.

In certain embodiments, an antibody or antigen-binding fragment thereof comprising, consisting essentially of, or consisting of a one or more of the VH and/or VL polypeptides described above specifically or preferentially binds to a DR6 polypeptide or fragment thereof, or a DR6 variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁷ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In other embodiments, an antibody or antigen-binding fragment thereof comprises, consists essentially of or consists of a VH polypeptide, and a VL polypeptide, where the VH polypeptide and the VL polypeptide, respectively are at least 80%, 85%, 90% 95% or 100% identical to reference VH and VL polypeptide amino acid sequences selected from the group consisting of SEQ ID NOs: 7 and 12, 17 and 22, 27 and 32, 37 and 42, 47 and 52, 57 and 62, 67 and 72, 77 and 82, 87 and 92, 97 and 102, 107 and 112, 117 and 122, 127 and 132, and 127 and 167. In certain embodiments, an antibody or antigen-binding fragment comprising these VH and VL polypeptides specifically or preferentially binds to DR6.

Any of the polypeptides described above can further include additional polypeptides, e.g., a signal peptide to direct secretion of the encoded polypeptide, antibody constant regions as described herein, or other heterologous polypeptides as described herein. Additionally, polypeptides include polypeptide fragments as described elsewhere. Additionally polypeptides include fusion polypeptide, Fab fragments, and other derivatives, as described herein.

Also, as described in more detail elsewhere herein, the present compositions include compositions comprising the polypeptides described above.

It will also be understood by one of ordinary skill in the art that DR6 antibody polypeptides as disclosed herein can be modified such that they vary in amino acid sequence from the naturally occurring binding polypeptide from which they were derived. For example, a polypeptide or amino acid sequence derived from a designated protein can be similar, e.g., have a certain percent identity to the starting sequence, e.g., it can be 60%, 70%, 75%, 80%, 85%, 90%, or 95% identical to the starting sequence.

Furthermore, nucleotide or amino acid substitutions, deletions, or insertions leading to conservative substitutions or changes at “non-essential” amino acid regions can be made. For example, a polypeptide or amino acid sequence derived from a designated protein can be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. A polypeptide or amino acid sequence derived from a designated protein can be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. In other embodiments, a polypeptide or amino acid sequence derived from a designated protein can be identical to the starting sequence except for two or fewer, three or fewer, four or fewer, five or fewer, six or fewer, seven or fewer, eight or fewer, nine or fewer, ten or fewer, fifteen or fewer, or twenty or fewer individual amino acid substitutions, insertions, or deletions. In certain embodiments, a polypeptide or amino acid sequence derived from a designated protein has one to five, one to ten, one to fifteen, or one to twenty individual amino acid substitutions, insertions, or deletions relative to the starting sequence.

Certain DR6 antibody polypeptides comprise, consist essentially of, or consist of an amino acid sequence derived from a human amino acid sequence. However, certain DR6 antibody polypeptides comprise one or more contiguous amino acids derived from another mammalian species. For example, a DR6 antibody can include a primate heavy chain portion, hinge portion, or antigen binding region. In another example, one or more murine-derived amino acids can be present in a non-murine antibody polypeptide, e.g., in an antigen binding site of a DR6 antibody. In another example, the antigen binding site of a DR6 antibody is fully murine. In certain therapeutic applications, DR6-specific antibodies, or antigen-binding fragments, variants, or analogs thereof are designed so as to not be immunogenic in the animal to which the antibody is administered.

In certain embodiments, a DR6 antibody polypeptide comprises an amino acid sequence or one or more moieties not normally associated with an antibody. Exemplary modifications are described in more detail below. For example, a single-chain fv antibody fragment can comprise a flexible linker sequence, or can be modified to add a functional moiety (e.g., PEG, a drug, a toxin, or a label).

An DR6 antibody polypeptide can comprise, consist essentially of, or consist of a fusion protein. Fusion proteins are chimeric molecules which comprise, for example, an immunoglobulin antigen-binding domain with at least one target binding site, and at least one heterologous portion, i.e., a portion with which it is not naturally linked in nature. The amino acid sequences can normally exist in separate proteins that are brought together in the fusion polypeptide or they can normally exist in the same protein but are placed in a new arrangement in the fusion polypeptide. Fusion proteins can be created, for example, by chemical synthesis, or by creating and translating a polynucleotide in which the peptide regions are encoded in the desired relationship.

The term “heterologous” as applied to a polynucleotide or a polypeptide, means that the polynucleotide or polypeptide is derived from a distinct entity from that of the rest of the entity to which it is being compared. For instance, as used herein, a “heterologous polypeptide” to be fused to a DR6 antibody, or an antigen-binding fragment, variant, or analog thereof is derived from a non-immunoglobulin polypeptide of the same species, or an immunoglobulin or non-immunoglobulin polypeptide of a different species.

Alternatively, in another embodiment, mutations can be introduced randomly along all or part of the immunoglobulin coding sequence, such as by saturation mutagenesis, and the resultant mutants can be incorporated into DR6 antibodies and screened for their ability to bind to the desired antigen, e.g., DR6.

### Fusion Polypeptides and Antibodies

DR6 polypeptides and antibodies can further be recombinantly fused to a heterologous polypeptide at the N- or C-terminus. For example, DR6 antagonist polypeptides or antibodies can be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

DR6 antagonist polypeptides and antibodies can be composed of amino acids joined to each other by peptide bonds or modified peptide bonds, i.e., peptide isosteres, and can contain amino acids other than the 20 gene-encoded amino acids.

DR6 antagonists include fusion proteins comprising, consisting essentially of, or consisting of a DR6 antagonist polypeptide or antibody fusion that inhibits DR6 function. In certain embodiments, the heterologous polypeptide to which the DR6 antagonist polypeptide or antibody is fused is useful for function or is useful to target the DR6 antagonist polypeptide or antibody. In certain embodiments, a soluble DR6 antagonist polypeptide, e.g., a DR6 polypeptide comprising the extracellular domain (corresponding to amino acids 1 to 349 or 41 to 349 of SEQ ID NO: 2), or any other soluble DR6 polypeptide fragment, variant or derivative described herein, is fused to a heterologous polypeptide moiety to form a DR6 antagonist fusion polypeptide. DR6 antagonist fusion proteins and antibodies can be used to accomplish various objectives, e.g., increased serum half-life, improved bioavailability, in vivo targeting to a specific organ or tissue type, improved recombinant expression efficiency, improved host cell secretion, ease of purification, and higher avidity. Depending on the objective(s) to be achieved, the heterologous moiety can be inert or biologically active. Also, it can be chosen to be stably fused to the DR6 antagonist polypeptide or antibody or to be cleavable, in vitro or in vivo. Heterologous moieties to accomplish these other objectives are known in the art.

As an alternative to expression of a DR6 antagonist fusion polypeptide or antibody, a chosen heterologous moiety can be preformed and chemically conjugated to the DR6 antagonist polypeptide or antibody. In most cases, a chosen heterologous moiety will function similarly, whether fused or conjugated to the DR6 antagonist polypeptide or antibody. Therefore, in the following discussion of heterologous amino acid sequences, unless otherwise noted, it is to be understood that the heterologous sequence can be joined to the DR6 antagonist polypeptide or antibody in the form of a fusion protein or as a chemical conjugate.

Due to its long half-life, wide in vivo distribution, and lack of enzymatic or immunological function, essentially full-length human serum albumin (HSA), or an HSA fragment, is commonly used as a heterologous moiety. Through application of methods and materials such as those taught in Yeh et al., Proc. Natl. Acad Sci. USA 89:1904-08 (1992) and Syed et al., Blood 89:3243-52 (1997), HSA can be used to form a DR6 antagonist fusion polypeptide or antibody or polypeptide/antibody conjugate that displays pharmacological activity by virtue of the DR6 moiety while displaying significantly increased in vivo stability, e.g., 10-fold to 100-fold higher. The C-terminus of the HSA can be fused to the N-terminus of the DR6 polypeptide. Since HSA is a naturally secreted protein, the HSA signal sequence can be exploited to obtain secretion of a soluble DR6 fusion protein into the cell culture medium when the fusion protein is produced in a eukaryotic, e.g., mammalian, expression system.

In one embodiment, a DR6 polypeptide is fused to a hinge and Fc region, i.e., the C-terminal portion of an Ig heavy chain constant region. Potential advantages of a DR6-Fc fusion include solubility, in vivo stability, and multivalency, e.g., dimerization. The Fc region used can be an IgA, IgD, or IgG Fc region (hinge-C_{H}2-C_{H}3). Alternatively, it can be an IgE or IgM Fc region (hinge-C_{H}2-C_{H}3-C_{H}4). An IgG Fc region is generally used, e.g., an IgG₁ Fc region or IgG₄ Fc region. In one embodiment, a sequence beginning in the hinge region just upstream of the papain cleavage site which defines IgG Fc chemically (i.e. residue 216, taking the first residue of heavy chain constant region to be 114 according to the Kabat system), or analogous sites of other immunoglobulins is used in the fusion. The precise site at which the fusion is made is not critical; particular sites are well known and can be selected in order to optimize the biological activity, secretion, or binding characteristics of the molecule. Materials and methods for constructing and expressing DNA encoding Fc fusions are known in the art and can be applied to obtain DR6 fusions without undue experimentation. Some methods described herein employ a DR6 fusion protein such as those described in Capon et al., U.S. Pat. Nos. 5,428,130 and 5,565,335.

In some embodiments, fully intact, wild-type Fc regions display effector functions that can be unnecessary and undesired in an Fc fusion protein. Therefore, certain binding sites can be deleted from the Fc region during the construction of the secretion cassette. For example, since coexpression with the light chain is unnecessary, the binding site for the heavy chain binding protein, Bip (Hendershot et al., Immunol. Today 8:111-14 (1987)), is deleted from the C_{H}2 domain of the Fc region of IgE, such that this site does not interfere with the efficient secretion of the immunofusin. Transmembrane domain sequences, such as those present in IgM, also are generally deleted.

In certain embodiments, the IgG₁ Fc region is used. Alternatively, the Fc region of the other subclasses of immunoglobulin gamma (gamma-2, gamma-3 and gamma-4) can be used in the secretion cassette. The IgG₁ Fc region of immunoglobulin gamma-1 includes at least part of the hinge region, the C_{H}2 region, and the C_{H}3 region. In some embodiments, the Fc region of immunoglobulin gamma-1 is a C_{H}2-deleted-Fc, which includes part of the hinge region and the C_{H}3 region, but not the C_{H}2 region. A C_{H}2-deleted-Fc has been described by Gillies et al., Hum. Antibod Hybridomas 1:47 (1990). In some embodiments, the Fc region of one of IgA, IgD, IgE, or IgM, is used.

DR6-Fc fusion proteins can be constructed in several different configurations. In one configuration the C-terminus of the DR6 polypeptide is fused directly to the N-terminus of the Fe hinge moiety. In a slightly different configuration, a short polypeptide, e.g., 2-10 amino acids, is incorporated into the fusion between the N-terminus of the DR6 moiety and the C-terminus of the Fc moiety. Such a linker provides conformational flexibility, which can improve biological activity in some circumstances. If a sufficient portion of the hinge region is retained in the Fc moiety, the DR6-Fc fusion will dimerize, thus forming a divalent molecule. A homogeneous population of monomeric Fc fusions will yield monospecific, bivalent dimers. A mixture of two monomeric Fc fusions each having a different specificity will yield bispecific, bivalent dimers.

Soluble DR6 polypeptides can be fused to heterologous peptides to facilitate purification or identification of the soluble DR6 moiety. For example, a histidine tag can be fused to a soluble DR6 polypeptide to facilitate purification using commercially available chromatography media.

### DR6 Antagonist Conjugates

DR6 antagonist polypeptides and antibodies include derivatives that are modified, i.e., by the covalent attachment of any type of molecule such that covalent attachment does not prevent the DR6 antagonist polypeptide or antibody from inhibiting the biological function of DR6. For example, but not by way of limitation, the DR6 antagonist polypeptides and antibodies can be modified e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of tunicamycin, etc. Additionally, the derivative can contain one or more non-classical amino acids.

Conjugation does not have to involve the N-terminus of a soluble DR6 polypeptide or the thiol moiety on serum albumin. For example, soluble DR6-albumin fusions can be obtained using genetic engineering techniques, wherein the soluble DR6 moiety is fused to the serum albumin gene at its N-terminus, C-terminus, or both.

Soluble DR6 polypeptides or DR6 antibodies can be polypeptides or antibodies wherein one or more polymers are conjugated (covalently linked) to the DR6 polypeptide or antibody. Examples of polymers suitable for such conjugation include polypeptides (discussed above), sugar polymers and polyalkylene glycol chains. Typically, but not necessarily, a polymer is conjugated to the soluble DR6 polypeptide or DR6 antibody for the purpose of improving one or more of the following: solubility, stability, or bioavailability.

The class of polymer generally used for conjugation to a DR6 antagonist polypeptide or antibody is a polyalkylene glycol. Polyethylene glycol (PEG) is most frequently used. PEG moieties, e.g., 1, 2, 3, 4 or 5 PEG polymers, can be conjugated to each DR6 antagonist polypeptide or antibody to increase serum half life, as compared to the DR6 antagonist polypeptide or antibody alone. PEG moieties are non-antigenic and essentially biologically inert. PEG moieties can be branched or unbranched.

The number of PEG moieties attached to the DR6 antagonist polypeptide or antibody and the molecular weight of the individual PEG chains can vary. In general, the higher the molecular weight of the polymer, the fewer polymer chains attached to the polypeptide. Usually, the total polymer mass attached to the DR6 antagonist polypeptide or antibody is from 20 kDa to 40 kDa. Thus, if one polymer chain is attached, the molecular weight of the chain is generally 20-40 kDa. If two chains are attached, the molecular weight of each chain is generally 10-20 kDa. If three chains are attached, the molecular weight is generally 7-14 kDa.

The polymer, e.g., PEG, can be linked to the DR6 antagonist polypeptide or antibody through any suitable, exposed reactive group on the polypeptide. The exposed reactive group(s) can be, e.g., an N-terminal amino group or the epsilon amino group of an internal lysine residue, or both. An activated polymer can react and covalently link at any free amino group on the DR6 antagonist polypeptide or antibody. Free carboxylic groups, suitably activated carbonyl groups, hydroxyl, guanidyl, imidazole, oxidized carbohydrate moieties and mercapto groups of the DR6 antagonist polypeptide or antibody (if available) also can be used as reactive groups for polymer attachment.

In a conjugation reaction, from about 1.0 to about 10 moles of activated polymer per mole of polypeptide, depending on polypeptide concentration, is typically employed. Usually, the ratio chosen represents a balance between maximizing the reaction while minimizing side reactions (often non-specific) that can impair the desired pharmacological activity of the DR6 antagonist polypeptide or antibody. In certain embodiments, at least 50% of the biological activity (as demonstrated, e.g., in any of the assays described herein or known in the art) of the DR6 antagonist polypeptide or antibody is retained. In further embodiments, nearly 100% is retained.

In some embodiments, the antibodies or polypeptides are fusion proteins comprising a DR6 antibody, or antigen-binding fragment, variant, or derivative thereof, and a heterologous polypeptide. The heterologous polypeptide to which the antibody is fused can be useful for function or is useful to target the DR6 polypeptide expressing cells. In one embodiment, a fusion protein comprises, consists essentially of, or consists of, a polypeptide having the amino acid sequence of any one or more of the VH regions of an antibody or the amino acid sequence of any one or more of the VL regions of an antibody or fragments or variants thereof, and a heterologous polypeptide sequence. In another embodiment, a fusion protein comprises, consists essentially of, or consists of a polypeptide having the amino acid sequence of any one, two, three of the VH-CDRs of a DR6-specific antibody, or fragments, variants, or derivatives thereof, or the amino acid sequence of any one, two, three of the VL-CDRs of a DR6-specific antibody, or fragments, variants, or derivatives thereof, and a heterologous polypeptide sequence. In one embodiment, the fusion protein comprises a polypeptide having the amino acid sequence of a VH-CDR3 of a DR6-specific antibody, or fragment, derivative, or variant thereof, and a heterologous polypeptide sequence, which fusion protein specifically binds to at least one epitope of DR6. In another embodiment, a fusion protein comprises a polypeptide having the amino acid sequence of at least one VH region of a DR6-specific antibody and the amino acid sequence of at least one VL region of a DR6-specific antibody or fragments, derivatives or variants thereof, and a heterologous polypeptide sequence. In one embodiment, the VH and VL regions of the fusion protein correspond to a single source antibody (or scFv or Fab fragment) which specifically binds at least one epitope of DR6. In yet another embodiment, a fusion protein comprises a polypeptide having the amino acid sequence of any one, two, three or more of the VH CDRs of a DR6-specific antibody and the amino acid sequence of any one, two, three or more of the VL CDRs of a DR6-specific antibody, or fragments or variants thereof, and a heterologous polypeptide sequence. In some embodiments, two, three, four, five, six, or more of the VH-CDR(s) or VL-CDR(s) correspond to single source antibody (or scFv or Fab fragment). Nucleic acid molecules encoding these fusion proteins are also encompassed.

### DR6 Polynucleotide Antagonists

Specific embodiments comprise a method of promoting nervous system cell survival by contacting the cells with a DR6 polynucleotide antagonist. The polynucleotide antagonist can be any polynucleotide that encodes a DR6-antagonist polypeptide. The polynucleotide antagonist can also be a nucleic acid molecule which specifically binds to a polynucleotide which encodes DR6. The human DR6 mRNA sequence is set forth below:

(SEQ ID NO: 171)
gccaccacgt gtgtccctgc gcccggtggc caccgactca
gtccctcgcc gaccagtctg ggcagcggag gagggtggtt
ggcagtggct ggaagcttcg ctatgggaag ttgttccttt
gctctctcgc gcccagtcct cctccctggt tctcctcagc
cgctgtcgga ggagagcacc cggagacgcg ggctgcagtc
gcggcggctt ctccccgcct gggcggccgc gccgctgggc
aggtgctgag cgcccctaga gcctcccttg ccgcctccct
cctctgcccg gccgcagcag tgcacatggg gtgttggagg
tagatgggct cccggcccgg gaggcggcgg tggatgcggc
gctgggcaga agcagccgcc gattccagct gccccgcgcg
ccccgggcgc ccctgcgagt ccccggttca gccatgggga
cctctccgag cagcagcacc gccctcgcct cctgcagccg
catcgcccgc cgagccacag ccacgatgat cgcgggctcc
cttctcctgc ttggattcct tagcaccacc acagctcagc
cagaacagaa ggcctcgaat ctcattggca cataccgcca
tgttgaccgt gccaccggcc aggtgctaac ctgtgacaag
tgtccagcag gaacctatgt ctctgagcat tgtaccaaca
caagcctgcg cgtctgcagc agttgccctg tggggacctt
taccaggcat gagaatggca tagagaaatg ccatgactgt
agtcagccat gcccatggcc aatgattgag aaattacctt
gtgctgcctt gactgaccga gaatgcactt gcccacctgg
catgttccag tctaacgcta cctgtgcccc ccatacggtg
tgtcctgtgg gttggggtgt gcggaagaaa gggacagaga
ctgaggatgt gcggtgtaag cagtgtgctc ggggtacctt
ctcagatgtg ccttctagtg tgatgaaatg caaagcatac
acagactgtc tgagtcagaa cctggtggtg atcaagccgg
ggaccaagga gacagacaac gtctgtggca cactcccgtc
cttctccagc tccacctcac cttcccctgg cacagccatc
tttccacgcc ctgagcacat ggaaacccat gaagtccctt
cctccactta tgttcccaaa ggcatgaact caacagaatc
caactcttct gcctctgtta gaccaaaggt actgagtagc
atccaggaag ggacagtccc tgacaacaca agctcagcaa
gggggaagga agacgtgaac aagaccctcc caaaccttca
ggtagtcaac caccagcaag gcccccacca cagacacatc
ctgaagctgc tgccgtccat ggaggccact gggggcgaga
agtccagcac gcccatcaag ggccccaaga ggggacatcc
tagacagaac ctacacaagc attttgacat caatgagcat
ttgccctgga tgattgtgct tttcctgctg ctggtgcttg
tggtgattgt ggtgtgcagt atccggaaaa gctcgaggac
tctgaaaaag gggccccggc aggatcccag tgccattgtg
gaaaaggcag ggctgaagaa atccatgact ccaacccaga
accgggagaa atggatctac tactgcaatg gccatggtat
cgatatcctg aagcttgtag cagcccaagt gggaagccag
tggaaagata tctatcagtt tctttgcaat gccagtgaga
gggaggttgc tgctttctcc aatgggtaca cagccgacca
cgagcgggcc tacgcagctc tgcagcactg gaccatccgg
ggccccgagg ccagcctcgc ccagctaatt agcgccctgc
gccagcaccg gagaaacgat gttgtggaga agattcgtgg
gctgatggaa gacaccaccc agctggaaac tgacaaacta
gctctcccga tgagccccag cccgcttagc ccgagcccca
tccccagccc caacgcgaaa cttgagaatt ccgctctcct
gacggtggag ccttccccac aggacaagaa caagggcttc
ttcgtggatg agtcggagcc ccttctccgc tgtgactcta
catccagcgg ctcctccgcg ctgagcagga acggttcctt
tattaccaaa gaaaagaagg acacagtgtt gcggcaggta
cgcctggacc cctgtgactt gcagcctatc tttgatgaca
tgctccactt tctaaatcct gaggagctgc gggtgattga
agagattccc caggctgagg acaaactaga ccggctattc
gaaattattg gagtcaagag ccaggaagcc agccagaccc
tcctggactc tgtttatagc catcttcctg acctgctgta
gaacataggg atactgcatt ctggaaatta ctcaatttag
tggcagggtg gttttttaat tttcttctgt ttctgatttt
tgttgtttgg ggtgtgtgtg tgtgtttgtg tgtgtgtgtg
tgtgtgtgtg tgtgtgtgtg tttaacagag aatatggcca
gtgcttgagt tctttctcct tctctctctc tctttttttt
ttaaataact cttctgggaa gttggtttat aagcctttgc
caggtgtaac tgttgtgaaa tacccaccac taaagttttt
taagttccat attttctcca ttttgccttc ttatgtattt
tcaagattat tctgtgcact ttaaatttac ttaacttacc
ataaatgcag tgtgactttt cccacacact ggattgtgag
gctcttaact tcttaaaagt ataatggcat cttgtgaatc
ctataagcag tctttatgtc tcttaacatt cacacctact
ttttaaaaac aaatattatt actattttta ttattgtttg
tcctttataa attttcttaa agattaagaa aatttaagac
cccattgagt tactgtaatg caattcaact ttgagttatc
ttgtatggtt ttcacctgga caccgtgtag aatgcttgat
tacttgtact cttcttatgc taatatgctc tgggctggag
aaatgaaatc ctcaagccat caggatttgc tatttaagtg
gcttgacaac tgggccacca aagaacttga acttcacctt
ttaggatttg agctgttctg gaacacattg ctgcactttg
gaaagtcaaa atcaagtgcc agtggcgccc tttccataga
gaatttgccc agctttgctt taaaagatgt cttgtttttt
atatacacat aatcaatagg tccaatctgc tctcaaggcc
ttggtcctgg tgggattcct tcaccaatta ctttaattaa
aaatggctgc aactgtaaga acccttgtct gatatatttg
caactatgct cccatttaca aatgtacctt ctaatgctca
gttgccaggt tccaatgcaa aggtggcgtg gactcccttt
gtgtgggtgg ggtttgtggg tagtggtgaa ggaccgatat
cagaaaaatg ccttcaagtg tactaattta ttaataaaca
ttaggtgttt gttaaaaaaa aaaaaaaaaa aaaaaaaaaa
aa

The DR6 polynucleotide antagonist prevents expression of DR6 (knockdown). In certain embodiments, the DR6 polynucleotide antagonist promotes nervous system cell survival or inhibits nervous system cell apoptosis. DR6 polynucleotide antagonists include, but are not limited to antisense molecules, ribozymes, siRNA, shRNA and RNAi. Typically, such binding molecules are separately administered to the animal (see, for example, O'Connor, J. Neurochem. 56:560 (1991), but such binding molecules can also be expressed in vivo from polynucleotides taken up by a host cell and expressed in vivo. See also Oligodeoxynucleotides as Antisense Inhibitors of Gene Expression, CRC Press, Boca Raton, Fla. (1988).

Polynucleotides can be DNA or RNA or chimeric mixtures or derivatives or modified versions thereof, single-stranded or double-stranded.

Polynucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al., Nucl. Acids Res. 16:3209 (1988), methylphosphonate oligonucleotides can be prepared by use of controlled pore glass polymer supports (Sarin et al., Proc. Natl. Acad. Sci. U.S.A. 85:7448-7451(1988)), etc.

Polynucleotide compositions further include catalytic RNA, or a ribozyme (See, e.g., PCT International Publication WO 90/11364, published Oct. 4, 1990; Sarver et al., Science 247:1222-1225 (1990). Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334:585-591 (1988). In certain embodiments, the ribozyme is engineered so that the cleavage recognition site is located near the 5′ end of the target mRNA; i.e., to increase efficiency and minimize the intracellular accumulation of non-functional mRNA transcripts.

Specific examples of polynucleotide molecules include siRNAs comprising the sequence AGAAACGGCUCCUUUAUUA (SEQ ID NO:160), GGAAGGACAUCUAUCAGUU (SEQ ID NO:161), GGCCGAUGAUUGAGAGAUU (SEQ ID NO:162), GCAGUUGGAAACAGACAAA (SEQ ID NO:163) or an antisense sequence present within or comprising: TTTTTTTTTTTTTTTTTTTTTTTTTTTTTAACAAACACCTAATGTTTATTAATAAA TTAGTACACTTGAAGGCATTITTCTGATATCGGTCCTTCACCACTACCCACAA ACCCCACCCACACAAAGGGAGTCCACGCCACCTTTGCATTGGAACCTGGCAA CTGAGCATTAGAAGGTACATTTGTAAATGGGAGCATAGTTGCAAATATATCA GACAAGGGTTCTTACAGTTGCAGCCATTTTTAATTAAAGTAAT (SEQ ID NO:172) can be used. In another embodiment, a combination of two or more siRNAs or antisense molecules can be used. For example, in one embodiment, a cocktail of four siRNAs can be used. The sequence of the control siRNA was:

| | |
|---|---|
| GGUGACAUGAUCGACAGCCAU. | (SEQ ID NO: 164) |

### DR6 Aptamer Antagonists

In another embodiment, the DR6 antagonist is an aptamer. An aptamer can be a nucleotide or a polypeptide which has a unique sequence, has the property of binding specifically to a desired target (e.g., a polypeptide), and is a specific ligand of a given target. Nucleotide aptamers include double stranded DNA and single stranded RNA molecules that bind to DR6. In certain embodiments, the DR6 aptamer antagonist promotes proliferation, differentiation, or survival of oligodendrocytes; promotes, oligodendrocyte-mediated myelination of neurons, or prevents demyelination, e.g., in a mammal.

Nucleic acid aptamers are selected using methods known in the art, for example via the Systematic Evolution of Ligands by Exponential Enrichment (SELEX) process. SELEX is a method for the in vitro evolution of nucleic acid molecules with highly specific binding to target molecules as described in e.g. U.S. Pat. Nos. 5,475,096, 5,580,737, 5,567,588, 5,707,796, 5,763,177, 6,011,577, and 6,699,843, incorporated herein by reference in their entirety. Another screening method to identify aptamers is described in U.S. Pat. No. 5,270,163 (also incorporated herein by reference). The SELEX process is based on the capacity of nucleic acids for forming a variety of two- and three-dimensional structures, as well as the chemical versatility available within the nucleotide monomers to act as ligands (form specific binding pairs) with virtually any chemical compound, whether monomeric or polymeric, including other nucleic acid molecules and polypeptides. Molecules of any size or composition can serve as targets.

Nucleotide aptamers can be modified (e.g., by modifying the backbone or bases or conjugated to peptides) as described herein for other polynucleotides.

Using the protein structure of DR6, screening for aptamers that act on DR6 using the SELEX process would allow for the identification of aptamers that inhibit DR6-mediated processes.

Polypeptide aptamers are peptides or small polypeptides that act as dominant inhibitors of protein function. Peptide aptamers specifically bind to target proteins, blocking their functional ability (Kolonin et al. (1998) Proc. Natl. Acad Sci. 95: 14,266-14,271). Peptide aptamers that bind with high affinity and specificity to a target protein can be isolated by a variety of techniques known in the art. Peptide aptamers can be isolated from random peptide libraries by yeast two-hybrid screens (Xu, C. W., et al. (1997) Proc. Natl. Acad Sci. 94:12,473-12,478) or by ribosome display (Hanes et al. (1997) Proc. Natl. Acad Sci. 94:4937-4942). They can also be isolated from phage libraries (Hoogenboom, H. R., et al. (1998) Immunotechnology 4:1-20) or chemically generated peptide libraries. Additionally, polypeptide aptamers can be selected using the selection of Ligand Regulated Peptide Aptamers (LiRPAs). See, e.g., Binkowski B F et al., (2005) Chem & Biol 12(7): 847-855, incorporated herein by reference. Although the difficult means by which peptide aptamers are synthesized makes their use more complex than polynucleotide aptamers, they have unlimited chemical diversity. Polynucleotide aptamers are limited because they utilize only the four nucleotide bases, while peptide aptamers would have a much-expanded repertoire (i.e., 20 amino acids).

Peptide aptamers can be modified (e.g., conjugated to polymers or fused to proteins) as described for other polypeptides elsewhere herein.

### P75 Antagonists

Antagonists of p75 include, for example, (i) p75 antagonists compounds; (ii) p75 antagonist polypeptides; (iii) p75 antagonist antibodies or fragments thereof; (iv) −75 antagonist polynucleotides; (v) p75 aptamers; and (vi) combinations of two or more of said p75 antagonists. In some embodiments, the p75 antagonist inhibits interaction of p75 with DR6.

P75 antagonists are known in the art, and one of ordinary skill in the art would know how to screen for and test p75 antagonists which would inhibit the interaction of p75 and DR6. For example, a cyclic decapeptide antagonist of p75 is described in Turner et al. J. Neuroscience Research 78: 193-199 (2004), which is herein incorporated by reference in its entirety.

### Vectors and Host Cells

Host-expression systems represent vehicles by which the coding sequences of interest can be produced and subsequently purified, but also represent cells which can, when transformed or transfected with the appropriate nucleotide coding sequences, express a DR6 and/or p75 antagonist polypeptide or antibody in situ. These include but are not limited to microorganisms such as bacteria (e.g., E. coli, B. subtilis) transformed with recombinant bacteriophage DNA, plasmid DNA or cosmid DNA expression vectors containing DR6 and/or p75 antagonist polypeptide or antibody coding sequences; yeast (e.g., Saccharomyces, Pichia) transformed with recombinant yeast expression vectors containing DR6 and/or p75 antagonist polypeptide or antibody coding sequences; insect cell systems infected with recombinant virus expression vectors (e.g., baculovirus) containing DR6 and/or p75 antagonist polypeptide or antibody coding sequences; plant cell systems infected with recombinant virus expression vectors (e.g., cauliflower mosaic virus. CaMV; tobacco mosaic virus, TMV) or transformed with recombinant plasmid expression vectors (e.g., Ti plasmid) containing DR6 and/or p75 antagonist polypeptide or antibody coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Bacterial cells such as Escherichia coli, or eukaryotic cells, e.g., for the expression of DR6 and/or p75 antagonist polypeptide or whole recombinant antibody molecules, are used for the expression of a recombinant DR6 and/or p75 antagonist polypeptide or antibody molecule. For example, mammalian cells such as Chinese hamster ovary cells (CHO), in conjunction with a vector such as the major intermediate early gene promoter element from human cytomegalovirus is an effective expression system for DR6 and/or p75 antagonist polypeptide or antibodies (Foecking et al., Gene 45:101 (1986); Cockett et al., Bio/Technology 8:2 (1990)).

Vectors comprising nucleic acids encoding DR6 and/or p75 antagonists, e.g., soluble polypeptides, antibodies, antagonist polynucleotides, or aptamers, can be used to produce antagonists. The choice of vector and expression control sequences to which such nucleic acids are operably linked depends on the functional properties desired, e.g., protein expression, and the host cell to be transformed.

Expression control elements useful for regulating the expression of an operably linked coding sequence are known in the art. Examples include, but are not limited to, inducible promoters, constitutive promoters, secretion signals, and other regulatory elements. When an inducible promoter is used, it can be controlled, e.g., by a change in nutrient status, or a change in temperature, in the host cell medium.

In one embodiment, a proprietary expression vector of Biogen IDEC, Inc., referred to as NEOSPLA (U.S. Pat. No. 6,159,730) can be used. This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression upon transfection in CHO cells, followed by selection in G418 containing medium and methotrexate amplification. Of course, any expression vector which is capable of eliciting expression in cells can be used. Examples of suitable vectors include, but are not limited to plasmids pcDNA3, pHCMV/Zeo, pCR3.1, pEFI/His, pIND/GS, pRc/HCMV2, pSV40/Zeo2, pTRACER-HCMV, pUB6N5-His, pVAX1, and pZeoSV2 (available from Invitrogen, San Diego, Calif.), and plasmid pCI (available from Promega, Madison, Wis.). Additional cell expression vectors are known in the art and are commercially available. Typically, such vectors contain convenient restriction sites for insertion of the desired DNA segment. Exemplary vectors include pSVL and pKSV-10 (Pharmacia), pBPV-1, pml2d (International Biotechnologies), pTDT1 (ATCC 31255), retroviral expression vector pMIG and pLL3.7, adenovirus shuttle vector pDC315, AAV vectors, pUC8, pUC9, pBR322 and pBR329 (BioRad), pPL and pKK223 (Pharmacia). Other exemplary vector systems are disclosed e.g., in U.S. Pat. No. 6,413,777.

### Gene Therapy

A DR6 and/or p75 antagonist can be produced in vivo in a mammal, e.g., a human patient, using a gene-therapy approach to treatment of a nervous-system disease, disorder or injury in which promoting survival, proliferation and differentiation of oligodendrocytes or promoting myelination of neurons would be therapeutically beneficial. This involves administration of a suitable DR6 and/or p75 antagonist-encoding nucleic acid operably linked to suitable expression control sequences. Generally, these sequences are incorporated into a viral vector. Suitable viral vectors for such gene therapy include an adenoviral vector, an alphavirus vector, an enterovirus vector, a pestivirus vector, a lentiviral vector, a baculoviral vector, a herpesvirus vector, an Epstein Barr viral vector, a papovaviral vector, a poxvirus vector, a vaccinia viral vector, adeno-associated viral vector and a herpes simplex viral vector. The viral vector can be a replication-defective viral vector. Adenoviral vectors that have a deletion in its E1 gene or E3 gene are typically used. When an adenoviral vector is used, the vector usually does not have a selectable marker gene.

### Pharmaceutical Compositions

DR6 and/or p75 antagonists can be formulated into pharmaceutical compositions for administration to mammals, including humans. The pharmaceutical compositions can comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions can be administered by any suitable method, e.g., parenterally, intraventricularly, intrathecally, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. As described previously, DR6 and/or p75 antagonists can act in the nervous system to promote survival and prevent apoptosis of nervous system cells. Accordingly, in certain embodiments, the DR6 and/or p75 antagonists are administered in such a way that they cross the blood-brain barrier. This crossing can result from the physico-chemical properties inherent in the DR6 and/or p75 antagonist molecule itself, from other components in a pharmaceutical formulation, or from the use of a mechanical device such as a needle, cannula or surgical instruments to breach the blood-brain barrier. Where the DR6 and/or p75 antagonist is a molecule that does not inherently cross the blood-brain barrier, e.g., a fusion to a moiety that facilitates the crossing, suitable routes of administration are, e.g., intrathecal or intracranial, e.g., directly into a chronic lesion of MS. Where the DR6 and/or p75 antagonist is a molecule that inherently crosses the blood-brain barrier, the route of administration can be by one or more of the various routes described below.

Sterile injectable forms of the compositions described herein can be aqueous or oleaginous suspension. These suspensions can be formulated according to techniques known in the art using suitable dispersing or wetting agents and suspending agents. The sterile, injectable preparation can also be a sterile, injectable solution or suspension in a non-toxic parenterally acceptable diluent or solvent, for example as a suspension in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms can also be used for the purposes of formulation.

Parenteral formulations can be a single bolus dose, an infusion or a loading bolus dose followed with a maintenance dose. These compositions can be administered at specific fixed or variable intervals, e.g., once a day, or on an “as needed” basis.

Certain pharmaceutical compositions can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of a DR6 and/or p75 antagonist that can be combined with the carrier materials to produce a single dosage form will vary depending upon the host treated, the type of antagonist used and the particular mode of administration. The composition can be administered as a single dose, multiple doses or over an established period of time in an infusion. Dosage regimens also can be adjusted to provide the optimum desired response (e.g., a therapeutic or prophylactic response).

### Methods of Treatment

In some cases, the methods described herein use a “therapeutically effective amount” or a “prophylactically effective amount” of a DR6 and/or p75 antagonist. Such a therapeutically or prophylactically effective amount can vary according to factors such as the disease state, age, sex, and weight of the individual. A therapeutically or prophylactically effective amount is also one in which any toxic or detrimental effects are outweighed by the therapeutically beneficial effects.

In one embodiment, DR6 antagonists improve the course of disease even when administered after a subject becomes symptomatic. For example, in one embodiment, the antagonist is administered in the early phase of ALS after motor neuron termini have begun to retract from muscle cells, i.e., after reduced muscle innervation can be demonstrated. In one embodiment, the DR6 antagonist is administered before DR6 expression (e.g., as measured by increased mRNA and/or increased protein) is upregulated in motor neurons. In one embodiment, the DR6 antagonist is administered after DR6 expression is upregulated in motor neurons. In another embodiment, the DR6 antagonist is administered after the disease has become symptomatic. For example, after the onset of twitching, cramping, or stiffness of muscles; muscle weakness affecting an arm or a leg; slurred and nasal speech; or difficulty chewing or swallowing.

In one embodiment, only lower motor neurons are involved, and the disease is called progressive muscular atrophy (PMA).

When only upper motor neurons are involved, the disease is called primary lateral sclerosis. For example, in one embodiment, the disease is restricted to bulbar muscles, in which case it is called progressive bulbar palsy (PBP).

A specific dosage and treatment regimen for any particular patient will depend upon a variety of factors, including the particular DR6 and/or p75 antagonist used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

DR6 and/or p75 antagonists can be generally administered systemically or directly to the nervous system, intracerebroventricularly, or intrathecally, e.g. into a chronic lesion. Compositions can be formulated so that a dosage of 0.001-10 mg/kg body weight per day of the DR6 and/or p75 antagonist is administered. In some embodiments, the dosage is 0.01-1.0 mg/kg body weight per day. In some embodiments, the dosage is 0.001-0.5 mg/kg body weight per day.

For treatment with a DR6 and/or p75 antagonist antibody, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, for example, at least 1 mg/kg. Doses intermediate in the above ranges can also be used. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered falls within the ranges indicated.

In certain embodiments, a subject can be treated with a nucleic acid molecule encoding a DR6 and/or p75 antagonist polynucleotide. Doses for nucleic acids range from about 10 ng to 1 g, 100 ng to 100 mg, 1 μg to 10 mg, or 30-300 μg DNA per patient. Doses for infectious viral vectors vary from 10-100, or more, virions per dose.

Supplementary active compounds also can be incorporated into compositions. For example, a soluble polypeptide or a fusion protein can be coformulated with and/or coadministered with one or more additional therapeutic agents.

The delivery methods encompass any suitable delivery method for a DR6 and/or p75 antagonist to a selected target tissue, including bolus injection of an aqueous solution or implantation of a controlled-release system. Use of a controlled-release implant reduces the need for repeat injections.

The DR6 and/or p75 antagonists described herein can be directly infused into the brain. Various implants for direct brain infusion of compounds are known and are effective in the delivery of therapeutic compounds to human patients suffering from neurological disorders. These include chronic infusion into the brain using a pump, stereotactically implanted, temporary interstitial catheters, permanent intracranial catheter implants, and surgically implanted biodegradable implants. See, e.g., Gill et al., supra; Scharfen et al., “High Activity Iodine-125 Interstitial Implant For Gliomas,” Int. J. Radiation Oncology Biol. Phys. 24(4):583-591 (1992); Gaspar et al., “Permanent 1251 Implants for Recurrent Malignant Gliomas,” Int. J. Radiation Oncology Biol. Phys. 43(5):977-982 (1999); chapter 66, pages 577-580, Bellezza et al., “Stereotactic Interstitial Brachytherapy,” in Gildenberg et al., Textbook of Stereotactic and Functional Neurosurgery, McGraw-Hill (1998); and Brem et al., “The Safety of Interstitial Chemotherapy with BCNU-Loaded Polymer Followed by Radiation Therapy in the Treatment of Newly Diagnosed Malignant Gliomas: Phase I Trial,” J. Neuro-Oncology 26:111-23 (1995).

The compositions can also comprise a DR6 and/or p75 antagonist dispersed in a biocompatible carrier material that functions as a suitable delivery or support system for the compounds. Suitable examples of sustained release carriers include semipermeable polymer matrices in the form of shaped articles such as suppositories or capsules. Implantable or microcapsular sustained release matrices include polylactides (U.S. Pat. No. 3,773,319; EP 58,481), copolymers of L-glutamic acid and gamma-ethyl-L-glutamate (Sidman et al., Biopolymers 22:547-56 (1985)); poly(2-hydroxyethyl-methacrylate), ethylene vinyl acetate (Langer et al., J. Biomed. Mater. Res. 15:167-277 (1981); Langer, Chem. Tech. 12:98-105 (1982)) or poly-D-(−)-3hydroxybutyric acid (EP 133,988).

In some embodiments, a DR6 and/or p75 antagonist is administered to a patient by direct infusion into an appropriate region of the brain. See, e.g., Gill et al., Nature Med 9: 589-95 (2003). Alternative techniques are available and can be applied to administer a DR6 and/or p75 antagonist. For example, stereotactic placement of a catheter or implant can be accomplished using the Riechert-Mundinger unit and the ZD (Zamorano-Dujovny) multipurpose localizing unit. A contrast-enhanced computerized tomography (CT) scan, injecting 120 ml of omnipaque, 350 mg iodine/ml, with 2 mm slice thickness can allow three-dimensional multiplanar treatment planning (STP, Fischer, Freiburg, Germany). This equipment permits planning on the basis of magnetic resonance imaging studies, merging the CT and MRI target information for clear target confirmation.

The Leksell stereotactic system (Downs Surgical, Inc., Decatur, Ga.) modified for use with a GE CT scanner (General Electric Company, Milwaukee, Wis.) as well as the Brown-Roberts-Wells (BRW) stereotactic system (Radionics, Burlington, Mass.) can be used for this purpose. Thus, on the morning of the implant, the annular base ring of the BRW stereotactic frame can be attached to the patient's skull. Serial CT sections can be obtained at 3 mm intervals though the (target tissue) region with a graphite rod localizer frame clamped to the base plate. A computerized treatment planning program can be run on a VAX 11/780 computer (Digital Equipment Corporation, Maynard, Mass.) using CT coordinates of the graphite rod images to map between CT space and BRW space.

The methods of treatment of nervous system disorders associated with increased cell death as described herein are typically tested in vitro, and then in vivo in an acceptable animal model, for the desired therapeutic or prophylactic activity, prior to use in humans. Suitable animal models, including transgenic animals, are will known to those of ordinary skill in the art. For example, in vitro assays to demonstrate the survival effect of the DR6 and/or p75 antagonists are described herein. The effect of the DR6 and/or p75 antagonists on apoptosis can be tested in vitro as described in the Examples. Finally, in vivo tests can be performed by creating transgenic mice which express the DR6 and/or p75 antagonist or by administering the DR6 and/or p75 antagonist to mice or rats in models.

The practices described herein will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning: A Laboratory Manual (3-Volume Set), J. Sambrook, D. W. Russell, Cold Spring Harbor Laboratory Press (2001); Genes VIII, B. Lewin, Prentice Hall (2003); PCR Primer, C. W. Dieffenbach and G. S. Dveksler, CSHL Press (2003); DNA Cloning, D. N. Glover ed., Volumes I and II (1985); Oligonucleotide Synthesis: Methods and Applications (Methods in Molecular Biology), P. Herdewijn (Ed.), Humana Press (2004); Culture of Animal Cells: A Manual of Basic Technique, 4th edition, R. I. Freshney, Wiley-Liss (2000); Oligonucleotide Synthesis, M. J. Gait (Ed.), (1984); Mullis et al. U.S. Pat. No. 4,683,195; Nucleic Acid Hybridization, B. D. Hames & S. J. Higgins eds. (1984); Nucleic Acid Hybridization, M. L. M. Anderson, Springer (1999); Animal Cell Culture and Technology, 2nd edition, M. Butler, BIOS Scientific Publishers (2004); Immobilized Cells and Enzymes: A Practical Approach (Practical Approach Series), J. Woodward, Irl Pr (1992); Transcription And Translation, B. D. Hames & S. J. Higgins (Eds.) (1984); Culture Of Animal Cells, R. I. Freshney, Alan R. Liss, Inc., (1987); Immobilized Cells And Enzymes, IRL Press, (1986); A Practical Guide To Molecular Cloning, 3rd edition, B. Perbal, John Wiley & Sons Inc. (1988); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155, Wu et al. (Eds.); Immunochemical Methods In Cell And Molecular Biology, Mayer and Walker, (Eds.), Academic Press, London (1987); Handbook Of Experimental Immunology, Volumes I-IV, D. M. Weir and C. C. Blackwell (Eds.), (1986); Immunology Methods Manual: The Comprehensive Sourcebook of Techniques (4 Volume Set), 1st edition, I. Lefkovits, Academic Press (1997); Manipulating the Mouse Embryo: A Laboratory Manual, 3rd edition, Cold Spring Harbor Laboratory Press (2002); and in Ausubel et al., Current Protocols in Molecular Biology, John Wiley and Sons, Baltimore, Md. (1989).

General principles of antibody engineering are set forth in Antibody Engineering: Methods and Protocols (Methods in Molecular Biology), B. L. Lo (Ed.), Humana Press (2003); Antibody engineering, R. Kontermann and S. Dubel (Eds.), Springer Verlag (2001); Antibody Engineering, 2nd edition, C. A. K. Borrebaeck (Ed.), Oxford Univ. Press (1995). General principles of protein engineering are set forth in Protein Engineering, A Practical Approach, Rickwood, D., et al. (Eds.), IRL Press at Oxford Univ. Press, Oxford, Eng. (1995). General principles of antibodies and antibody-hapten binding are set forth in: Antibodies: A Laboratory Manual, E. Harlow and D. Lane, Cold Spring Harbor Laboratory Press (1988); Nisonoff, A., Molecular Immunology, 2nd edition, Sinauer Associates, Sunderland, Mass. (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al. (Eds.), Immunochemical Protocols (Methods in Molecular Biology), 2nd edition, J. D. Pound (Ed.), Humana Press (1998), Weir's Handbook of Experimental Immunology, 5th edition, D. M. Weir (Ed.), Blackwell Publishers (1996), Methods in Cellular Immunology, 2nd edition, R. Fernandez-Botran, CRC Press (2001); Basic and Clinical Immunology, 8th edition, Appleton & Lange, Norwalk, Conn. (1994) and Mishell and Shiigi (Eds.), Selected Methods in Cellular Immunology, W.H. Freeman and Co., New York (1980).

Standard reference works setting forth general principles of immunology include Current Protocols in Immunology, John Wiley & Sons, New York; Klein, J.; Kuby Immunology, 4th edition, R. A. Goldsby, et al., H. Freeman & Co. (2000); Basic and Clinical Immunology, M. Peakman, et al., Churchill Livingstone (1997); Immunology, 6th edition, I. Roitt, et al., Mosby, London (2001); Cellular and Molecular Immunology, 5th edition; A. K. Abbas, A. H. Lichtman, Elsevier—Health Sciences Division (2005); Immunology Methods Manual: The Comprehensive Sourcebook of Techniques (4 Volume Set), 1st edition, I. Lefkovits, Academic Press (1997) Immunology, 5th edition, R. A. Goldsby, et al., W. H. Freeman (2002); Monoclonal Antibodies: Principles and Practice, 3rd Edition, J. W. Goding, Academic Press (1996); Immunology: The Science of Self-Nonself Discrimination, John Wiley & Sons, New York (1982); Kennett, R., et al. (Eds.), Monoclonal Antibodies, Hybridoma: A New Dimension in Biological Analyses, Plenum Press, New York (1980); Campbell, A., “Monoclonal Antibody Technology” in Burden, R., et al. (Eds.), Laboratory Techniques in Biochemistry and Molecular Biology, Vol. 13, Elsevere, Amsterdam (1984).

All of the references cited above are incorporated herein by reference in their entireties.

### EXAMPLES

The following materials and methods were used throughout the Examples:

In Situ Hybridization:

Frozen spinal cords sections were obtained from 65-day SOD1^{G93A} mice or aged matched control animals. Human ALS spinal cords tissues were purchased from Tissue Solutions. Frozen sections were probed with digoxigenin-labelled DR6 anti-sense probe (5′-TAATACGACTCACTATAGGGGCTGGTGGGTAAGTTGTGGT-3′; SEQ ID NO:173) and sense RNA probe (5′-ATITAGGTGACACTATAGAACTCGCGGTACCTTCTCTGAC-3′; SEQ ID NO:174)). Three SOD1^{G93A} animals and four human ALS tissues were used totally. DR6⁺ motor neurons located in each anterior horn of spinal cords were counted.

Western Blots:

Western blotting was carried out using mouse antibody to DR6 (Biogen Idec), antibody to cleaved caspase 3 (cell signaling), phosphorylated and total Akt (cell signaling), and rabbit antibody to β-actin (Sigma). Band intensities were quantified by densitometry.

Motor Neuron Survival:

Rat motor neurons were isolated from E18 Sprague Dawley rat (Charles River) spinal cords using multiple discontinuous density gradients of NycoPrep™. ES cells derived human motor neurons were purchased from Amaxa. Neurons were then plated in 4-well chamber slides coated with poly-D lysine and laminin at the density of 3-5×10⁴/well. After 24 h incubation at 37° C. in humidified air with 5% C02, neurons were treated with 0.5 mM sodium arsenite for 30 minutes. Cells were then washed with Neurobasal medium 3 times, and motor neuron culture media were added containing the indicated concentration of anti-DR6 antibody 5D10 or control antibody MOPC21. The cultures were continued for an additional 24 h, then were fixed with 4% PFA for immunocytochemistry study. Cells were co-stained with neurofilament (NF Millipore). Live motor neurons were identified by NF positive cells, and counted under microscope. At least 10 randomly selected fields were counted at each treatment condition. Axon length was measured using openlab software.

Astrocyte Motor Neuron Co-Culture:

Mouse astrocytes were isolated from brain of non-transgenic or SOD1^{G93A} transgenic mouse at the age of 2 months. Briefly, mouse forebrain was dissected out and put in cold HBSS, then the tissue was chopped with sterile razor blade into ˜1 mm chunks. Add DNAase and trypsin in HBSS to digest the tissue at 37° C. for 15 minutes, spin down at 800 rpm, discard supernatant. Add DMEM plus 10%/o FBS, and triturate tissue with glass pipette until homogeneous, then let tissue settle for 5 minutes, pass suspension through 70 um sieve (Falcon) and collect in tube. Repeat the trituration step once. Put the cell suspension in cell culture flask (˜10⁷ cells/flask), and grow it at 37° C. in humidified air with 5% C02 until confluence. Trypsinized the cells, and plated at the density of 5×10⁴/well in 4-well chamber slides coated with poly-D lysine and laminin. After 24 hours incubation at 37° C. and 5% C02, purified rat motor neurons were added on top of astrocytes at the density of 5×10⁴/well, together with indicated concentration of Anti-DR6 or control antibody. The cultures were for additional 7 days, then stained with NF as described above.

Animal and Therapeutic Regimens:

The transgenic SOD1^{G93A} mice used for these studies were the mixed hybrid, high-copy strain (B6SJL-Tg (SOD1-G93A) 1Gur/J, stock no. 002726) from the Jackson Laboratory. Mice were shipped at 6 weeks of age and maintained at the facility of Biogen Idec. All animal protocols were in accordance with US National Institutes of Health guidelines and approved by the Biogen Idec Institutional Committee. For survival study, mice were randomly assigned into two groups, 20 males and 20 females each group. Mice were treated with 6 mg/kg DR6 antagonist antibody 5D10 or control antibody MOPC21 twice per week, given intraperitoneally in volumes of 10 ml/kg beginning at 42 days of age and continuing until death. Body weight and disease onset were monitored daily to access disease progression and survival duration. Disease onset was defined as slightly impaired initiation of movement. Endpoint was defined as animal unable to right itself within 30 s when placed on either or both side(s).

For immunohistochemistry study, mice were treated with 6 mg/kg anti-DR6 antibody or control antibody twice (once) per week, given intraperitoneally in volumes of 10 ml/kg beginning at 42 days of age and continuing to the time point when tissues were harvest (at the age of day 60, 80 and 100). At each time point, each group had 3-6 littermate matched mice. SOD1^{G93A}/DR6−/− and SOD1^{G93A}/DR6+/+ mice were generated by crossing transgenic SOD1^{G93A} mice with DR6−/− mice. Genotype was confirmed with quantitative PCR.

Genotyping:

Quantitative PCR was used to confirm SOD1^{G93A} transgene copy number relative to the endogenous gene IL-2. After excluding mice having very low copy number, all mice had 23±4 copies of the transgene. Genotype of DR6 locus was confirmed.

Rota-Rod Analysis:

Motor coordination was measured with a rota-rod (UGO Basile). Mice were acclimatized on the rota-rod prior to data collection (at the age of 11 week). The data were collected at the age of week 12, 14 and 16. To perform the test, mice were placed on a rod rotating at an accelerating speed (starting from 2 rpm to 40 rpm in 3 minutes), and the test was continued to a maximum of 4 minutes or until the mice fell from the rod. The time that a mouse stayed on the rod was recorded and presented as latency to fall (second) for each mouse. The test was repeated 3 times in a day for each mouse. Out of 3 tests, the longest time for each mouse was used. At each time point, n=20 in control or treatment group.

Immunohistochemistry:

Toluidine blue staining of sciatic nerve was used to determine myelinated axons. Tissue sections were fixed in 4% (wt/vol) paraformaldehyde and processed. Rabbit antibody to Glial fibrillary acidic protein (GFAP, Dako) was used for immunohistochemistry. Standard Nissl stain on spinal cord motor neuron was used. For quantification of motor neuron and GFAP, at least 3 sections/animal, 3 animals/group were used. For gastrocnemius muscle and diaphragm neuromuscular junctions, 20 μm thick frozen sections were used for staining. Sections were stained with monoclonal antibodies to synaptic vesicle protein 2 (SV2, Iowa Developmental Hybridoma Bank), Alexa594 α-bungarotoxin (BuTx, Life technologies). Secondary antibody was Alexa488-conjugated goat anti-mouse (Life technologies). Single plane or z-stack images were collected using VS120 scanner (Olympus). Neuromuscular junctions were defined as “completely innervated” if there was complete overlap of the presynaptic marker (SV2, green) with acetylcholine receptor (AChR, red), revealed by BuTx staining; or “completely denervated” if there was no overlap; or “partially denervated” if there was partially overlap. 100 neuromuscular junctions from each animal were evaluated. Data was presented as percentage in each category. For quantification of NMJs, 6 littermate matched animals/group were used.

Statistical Analysis:

GraphPad Prism software was used for statistical analysis. For survival study, the time-to-event analysis was conducted. For all other studies, comparison of mean values was conducted with unpaired Student's t tests or one-way ANOVA with Tukey correction. In all analyses, statistical significance was determined at the 5% level (P<0.05).

### Example 1

### DR6 is Upregulated in ALS Postmortem Samples and SOD1^{G93A} Mice Spinal Cords

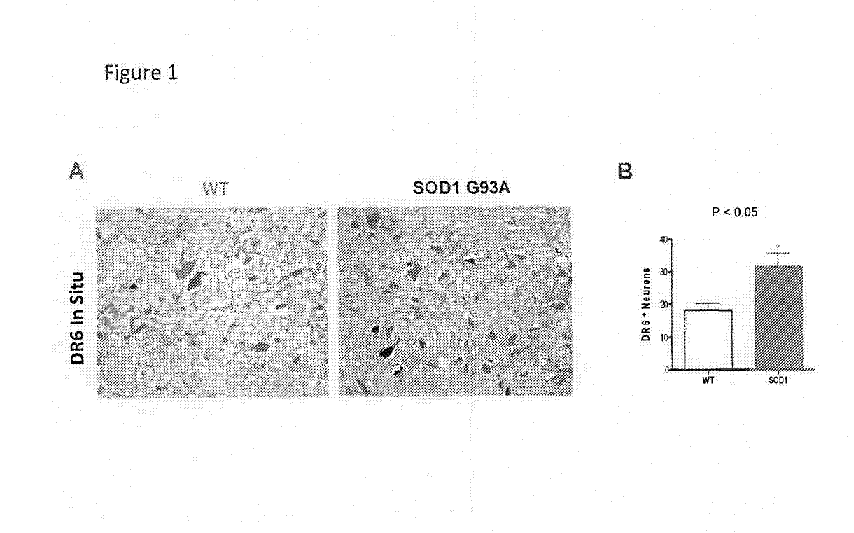

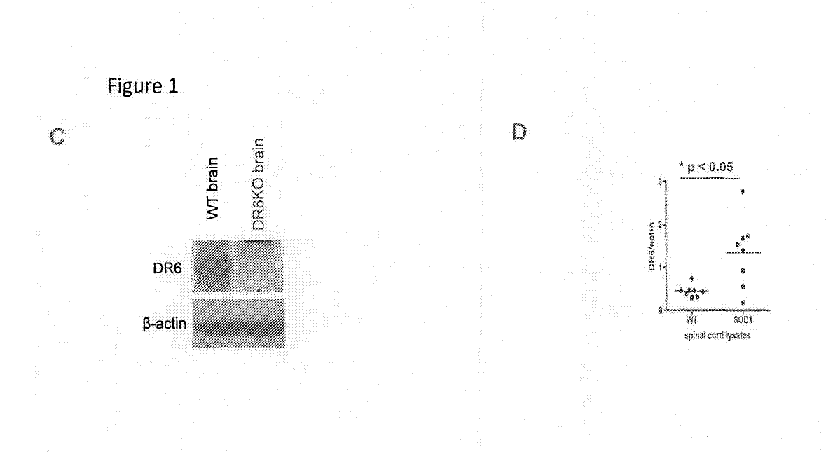

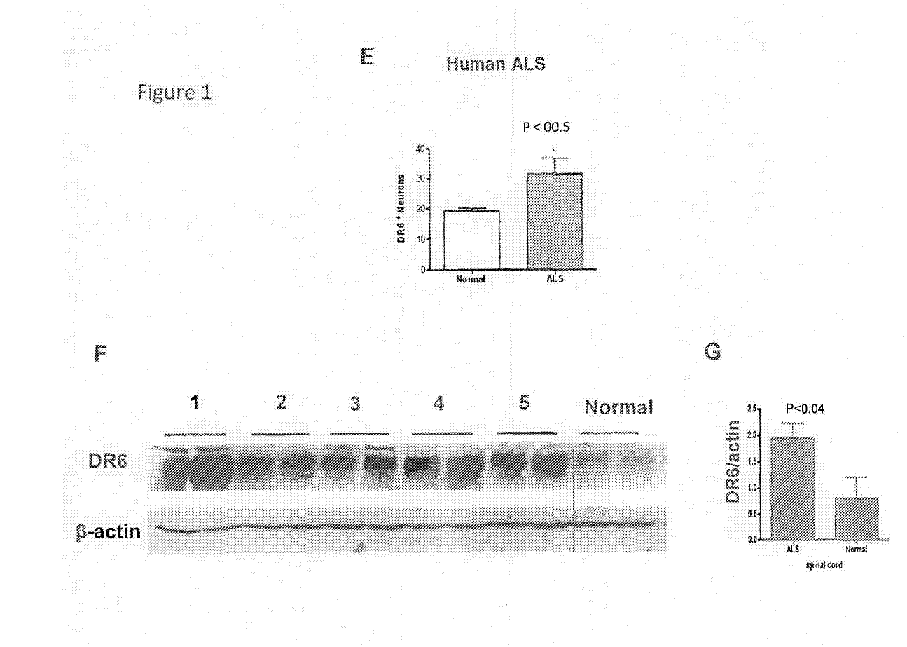

DR6 is broadly expressed by developing neurons, including motor neurons. Since ALS is a motor neuron disease, investigations were preformed to determine if DR6 was involved in ALS pathology. First, DR6 levels were determined in SOD1^{G93A} transgenic mice, the most characterized animal model for ALS. Using in situ hybridization to quantify DR6 mRNA levels in the ventral horn region of the lumbar spinal cord of SOD1^{G93A} mice, DR6 antisense RNA strongly stained motor neurons, as evident from their characteristic morphology (FIG. 1A). There were 1.7 fold more DR6 positive neurons in SOD1^{G93A} than in aged-matched non-transgenic animals (FIG. 1B n=3/group). DR6 positive SOD1^{G93A} neurons were smaller and stained more intensively than control (FIG. 1A), suggesting that DR6 expression is upregulated in pathological motor neurons.

To determine if DR6 protein levels are also increased in spinal cord of SOD1^{G93A} mice, immunohistochemistry (IHC) and Western blotting (WB) were performed using anti-DR6 antibody, 6A12. The specificity of this antibody was confirmed using DR6-null mice tissue. DR6 positive neurons and DR6 protein were detected in WT, but not in DR6-null brain by IHC and WB (WB shown in FIG. 1C). The number of DR6 positive neurons was significantly more (p=0.037) in SOD1^{G93A} spinal cord compared to control. Quantitative WB analysis showed a two-fold increase DR6 protein levels in SOD1^{G93A} spinal cord (FIG. 1D).

Next it was investigated if DR6 expression was upregulated in human ALS postmortem cervical spinal cord tissue by in situ hybridization and WB. A 1.6-fold increase in DR6 positive motor neurons was observed in ALS samples compared to aged matched non-disease controls by in situ hybridization (FIG. 1E). WB showed a 2-fold increase in DR6 protein levels in the ALS samples (FIG. 1F, G). The presence of elevated DR6 mRNA and protein levels in spinal cord of SOD1^{G93A} mice and human ALS postmortem samples, suggested that DR6 may contribute to ALS pathology.

### Example 2

### Blocking DR6 Promotes Motor Neuron Survival In Vitro

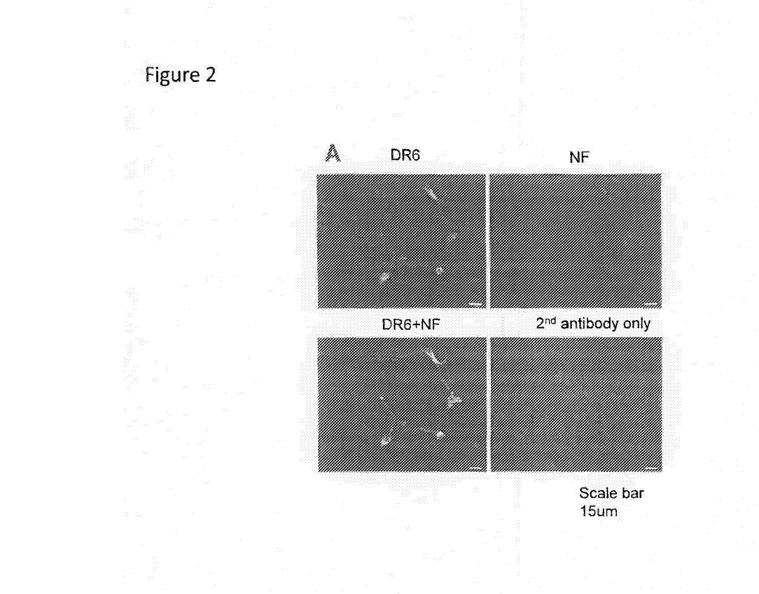

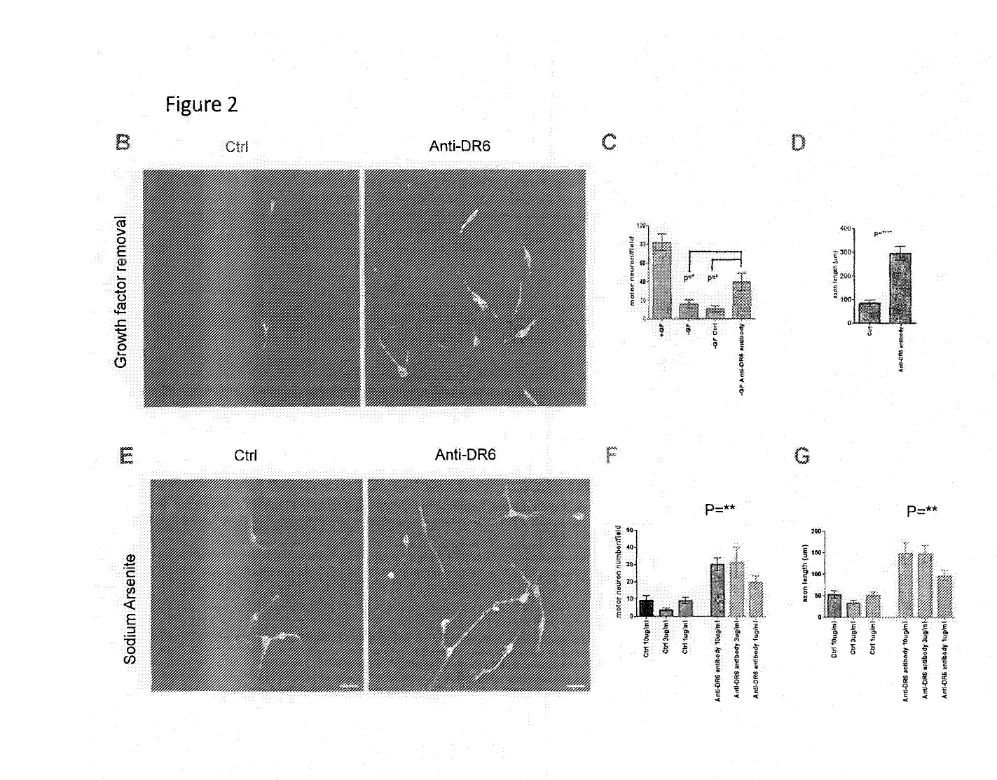

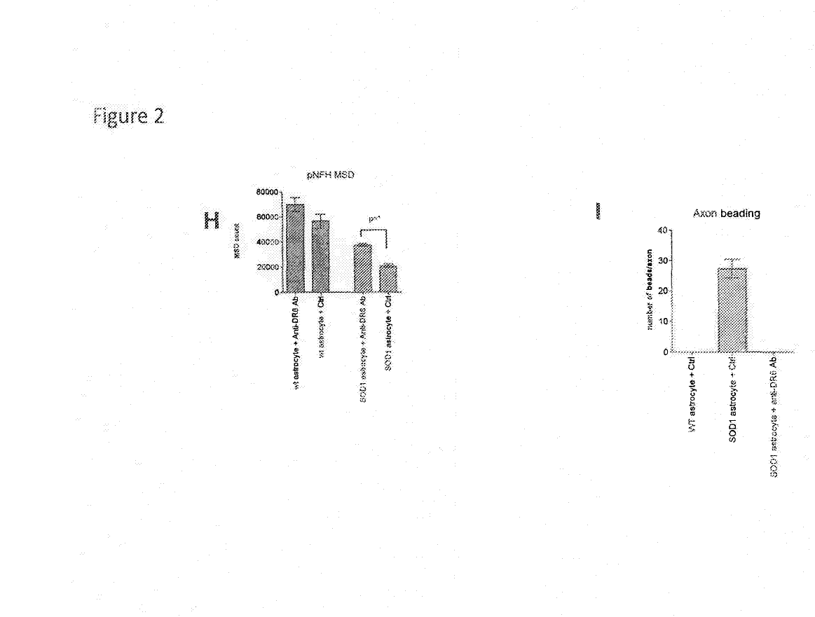

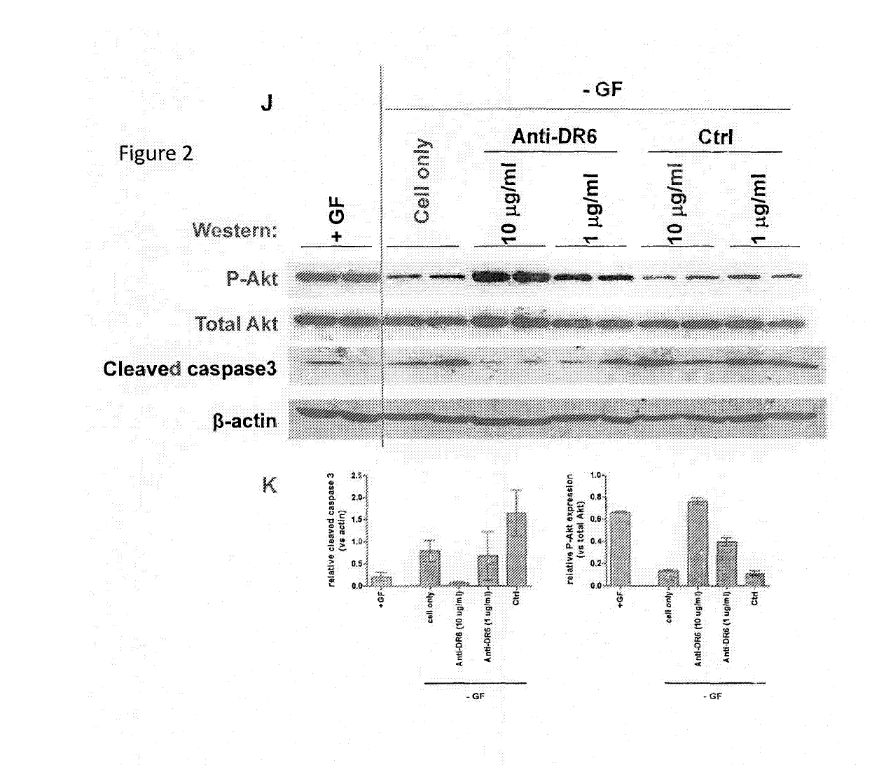

DR6 was previously reported to induce developmental neuronal cell death. Based on this information in combination with the data that DR6 is upregulated in SOD1^{G93A} mice and human ALS postmortem samples, it was hypothesized that DR6 may play a role in motor neuron death, and blocking DR6 could promote motor neuron survival in cell culture. To test this hypothesis, DR6 expression was determined in cultured human motor neurons. Immunocytochemistry analysis (ICC) of human motor neurons revealed anti-DR6 antibody 6A12, but not control antibody, co-stained motor neuron with anti-neurofilament (NF) antibody (FIG. 2A). Staining occurred in both the cell body and axons.

It was next determined if blocking DR6 protected motor neuron from death using three methods: growth factor removal, sodium arsenite (induced mitochondrion oxidative stress), and astrocyte (SOD1^{G93A}) induced cytotoxicity in motor neuron/astrocyte co-culture. Growth factor removal led to a 4-fold reduction in the number of surviving neurons, while anti-DR6 antibody treatment following growth factor removal increased the number of surviving neurons by 2-fold (FIG. 2B, C). Axon length in anti-DR6 antibody-treated neurons was 3-fold longer than control treated cells (FIG. 2B, D). The anti-DR6 antibody also increased motor neuron survival following sodium arsenite treatment in a dose dependent manner (FIG. 2E-G), with a maximum 3-fold increase in cell number at 10 μg/ml. Blocking DR6 by the same anti-DR6 antibody, also promoted rat motor neuron survival. Third, anti-DR6 antibody protected motor neurons from astrocyte-induced toxicity (FIG. 2H, I). In this study, purified astrocytes from the brains of 2 month old SOD1^{G93A} transgenic or control mice were co-cultured with purified human or rat motor neurons. Motor neurons were visualized by ICC using anti-NF antibody. The toxic effect of the SOD1^{G93A} astrocytes to motor neurons was clearly evident by the large reduction in the numbers of neurons in SOD1^{G93A} astrocyte-neuron co-culture than normal astrocytes-neuron co-culture (data not shown). Similarly, quantification of NF levels in the cultures revealed a >2-fold reduction of NF level in SOD1^{G93A} astrocyte-neuron co-cultures (FIG. 2H). In SOD1^{G93A} astrocyte co-culture, there were many beaded structures along the axons visible by ICC, indicating axon degeneration, while these structures were rarely seen in control co-cultures (data not shown). When anti-DR6 antibody was added to the SOD1^{G93A} astrocyte-neuron cultures, there was a 2-fold increase of motor neuron number and NF levels, and complete disappearance of axon beading (FIG. 2H, I), thus demonstrating that anti-DR6 antibody inhibited SOD1^{G93A} astrocyte induced motor neuron cytotoxicity.

To investigate the mechanism of action by which the anti-DR6 antibody treatment promoted survival of motor neurons, the levels of cleaved caspase 3 (casp3, apoptosis) and Akt phosphorylation (p-Akt, survival) were quantitiated by WB. Growth factor withdrawal led to a 2-fold increase of the active form of caspase 3 (cleaved caspase 3), and a 3-fold decrease of p-Akt (FIG. 2J, K). In contrast, anti-DR6 antibody treatment decreased the level of cleaved caspase 3, and increase the level of p-Akt in a dose-dependent manner (FIG. 2K). These data demonstrated that blocking DR6 promotes motor neuron survival likely through inhibiting casp3 activation, and promoting p-Akt survival signaling pathway.

### Example 3

### Blocking DR6 Promotes Survival and Functional Recovery in SOD1^{G93A} Mice

The effects of blocking DR6 on survival and functional recovery in the SOD1^{G93A} mice was determined using DR6 antagonist monoclonal antibody 5D10, the same antibody used previously in multiple sclerosis animal models. Mice were treated with 6 mg/kg 5D10 or control antibody MOPC21 intraperitoneally twice per week, beginning on day 42. Body weight, onset of clinical symptom, survival duration, and functional improvement by Rota-rod were monitored.

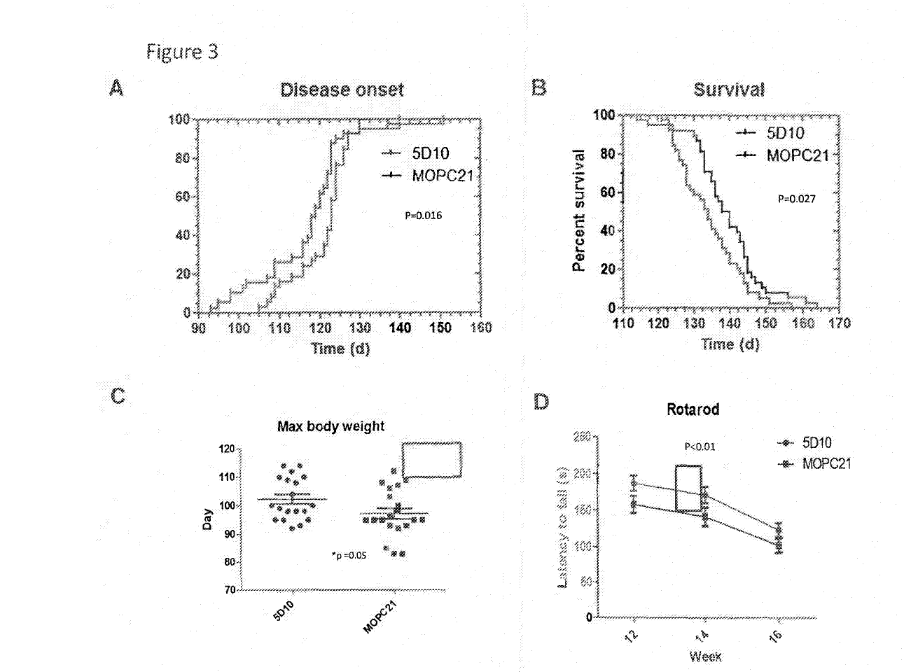

Disease onset was defined as slightly impaired initiation of movement based on Jackson Lab's criteria. Endpoint was defined as animal unable to right itself within 30 s when placed on either or both side(s). 5D10 treatment significantly delayed disease onset by 4 days, with median times to onset of 123 and 119 days in treatment and control groups, respectively (FIG. 3A; Log-rank (Mantel-Cox) test. p=0.016). 5D10 treatment also significantly increased survival by 5 days, with survival median times of 139 and 134 days in treatment and control groups, respectively (FIG. 3B; p=0.027). Interestingly, the beneficial effect of 5D10 treatment on male SOD1^{G93A} mice was better, with a 6-day delay in disease onset (data not shown), and an 8.5-day increase of survival (data not shown). SOD1^{G93A} mice lose body weight, which can be used to indicate if treatment impacts disease progression. Animals in 5D10 treatment group had significantly higher peak body weights than mice in control group (FIG. 3C).

Rota-rod analysis was performed to evaluate the balance and motor coordination in the mice. 5D10 treatment significantly improved SOD1^{G93A} mice rota-rod performance at all three time points assessed (days 84, 98, and 112, FIG. 3D). This study demonstrates that blocking DR6 function promotes survival and functional recovery in the SOD1^{G93A} model of ALS.

### Example 4

### Blocking DR6 Promotes Tissue Integrity in SOD1^{G93A} Mice

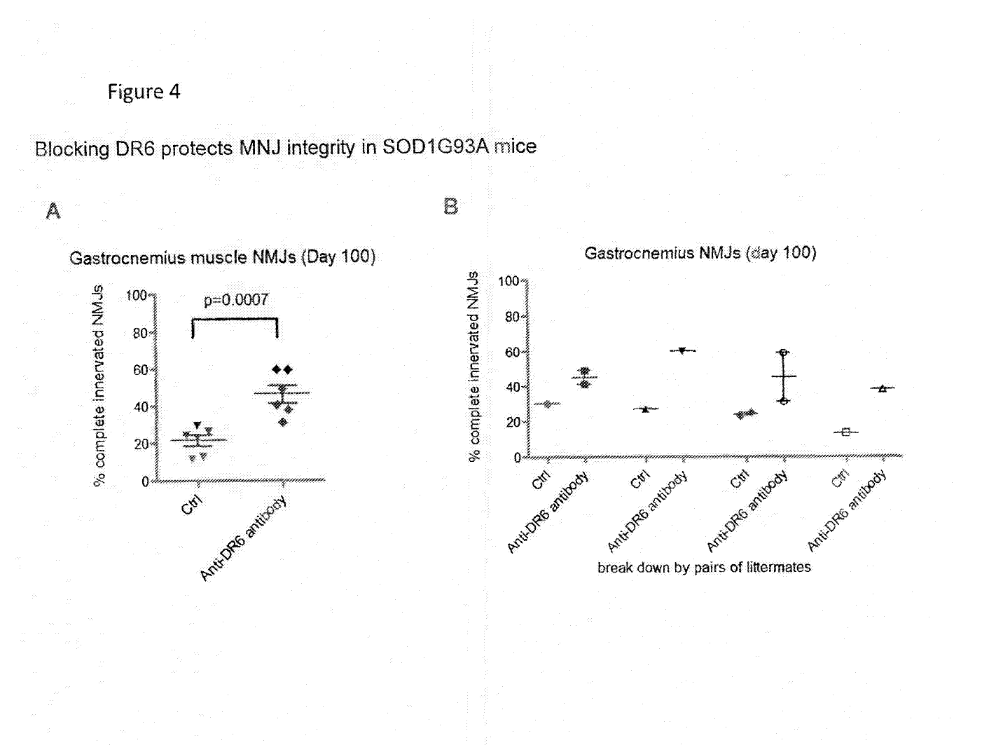

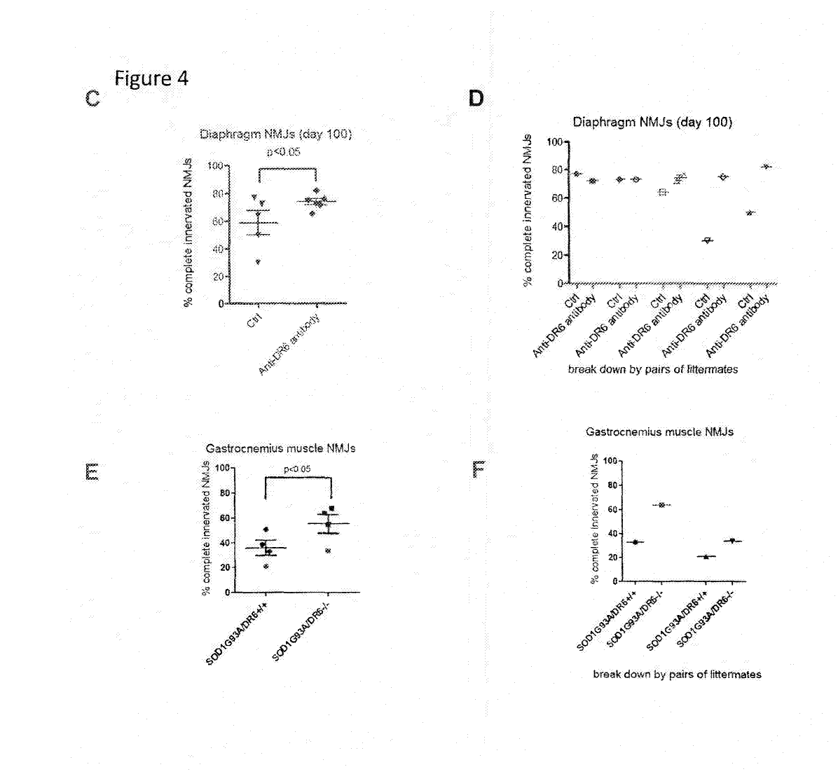

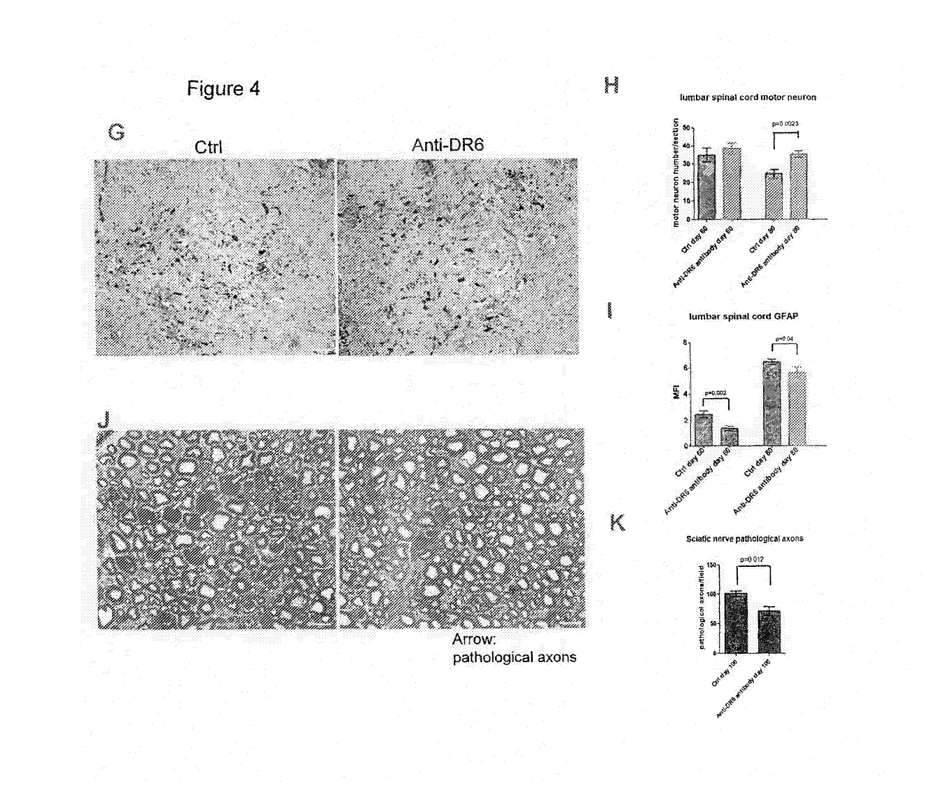

It was next determined if the therapeutic effects of the anti-DR6 antibody correlated with decreased tissue pathology. Neuromuscular junctions (NMJs) denervation is an early pathological event in SOD1^{G93A} mice. Gastrocnemius muscle was cut longitudinally and stained by IHC. Pre-synaptic nerve and post-synaptic muscle of NMJs were visualized with antibody against synaptic vesicle protein 2 (SV2), and α-bungarotoxin (BuTx), which binds nicotinic acetylcholine receptor (nAChR), respectively. NMJs were divided in three categories: “complete innervated” (healthy functional NMJs with a complete overlap of SV2 staining with BuTx staining); “complete denervated” (no overlap); or “partial denervated” if there was partially overlap. 100 neuromuscular junctions from each animal were evaluated. Data are presented as percentage of complete innervated NMJs. At day 100 (the time of disease onset), there was extensive denervation of NMJs in control treated SOD1^{G93A} mice as only 20% of NMJs remains completely innervated (FIG. 4A). Anti-DR6 antibody treatment significantly increased the percentage of complete innervated NMJs by 2-fold. Because the SOD1^{G93A} mouse is a mixed B6/SJL hybrid strain, littermates were matched across different experimental groups to minimize genetic background variation. When individual pairs of littermates were examined, anti-DR6 antibody treatment preserved NMJs in all pairs of matched littermates (FIG. 4B). Diaphragm NMJs were also examined, since some ALS patients die of lethal respiratory failure. Anti-DR6 antibody treatment significantly increased complete innervated NMJs by 28% (FIG. 4C). In 5 pairs of matched littermates, 3 pairs showed little denervation in either group (˜20% denervation vs. ˜80% denervation in gastrocnemius). The other 2 control treated mice showed severe denervation, while anti-DR6 treatment significantly increased complete innervated NMJs by ˜2 fold in these 2 pairs (FIG. 4D).

Nissl staining of lumbar spinal cord sections was used to determine the effect of 5D10 treatment on motor neuron number in SOD1^{G93A} mice. From day 60 to day 80, there was a 30% drop in motor neuron number in control treated mice (FIG. 4G, H). In contrast, anti-DR6 treatment prevented the loss as evident by 30% more motor neurons at day 80 versus in the control group (FIG. 4H, p=0.0023, two-tailed T test). Concomitant with motor neuron loss, glial fibrillary acidic protein (GFAP) levels, a measure of astrocyte activation, were elevated by 2.5-fold in control treated mice from day 60 to 80, (FIG. 4I). GFAP levels were 2-fold lower on day 60 in 5D10 treated group, and 20% lower on day 80 (FIG. 4I). In addition, on day 100, anti-DR6 treated mice showed significantly less sciatic nerve axon pathology than control treated mice when visualized by toluidine blue staining on 1 m sections (FIG. 4J, K). No sciatic nerve axon pathology was observed in mice on day 60 or 80 in either 5D10 or control treated groups.

Taken together the data demonstrated that, in addition to survival and functional improvement, 5D10 treatment preserved MNJ integrity, promoted motor neuron survival, decreased gliosis, and protected sciatic nerve integrity in SOD1^{G93A} mice.

### Example 5

### Cloning Murine Anti-Human DR6 Monoclonal Antibody Variable Domains

Total cellular RNA from murine hybridoma cells was prepared using a Qiagen RNeasy mini kit following the manufacturer's recommended protocol. cDNAs encoding the variable regions of the heavy and light chains were cloned by RT-PCR from total cellular RNA, using random hexamers for priming of first strand cDNA. For PCR amplification of the murine immunoglobulin variable domains with intact signal sequences, a cocktail of degenerate forward primers hybridizing to multiple murine immunoglobulin gene family signal sequences and a single back primer specific for the 5′ end of the murine constant domain were used. The PCR products were gel-purified and subcloned into Invitrogen's pCR2.1TOPO vector using their TOPO cloning kit following the manufacturer's recommended protocol. Inserts from multiple independent subclones were sequenced to establish a consensus sequence. Deduced mature immunoglobulin N-termini were identical to those determined by Edman degradation of the purified immunoglobulins from the hybridomas. Assignment to specific subgroups is based upon BLAST analysis using consensus immunoglobulin variable domain sequences from the Kabat database. CDRs are designated using the Kabat definitions.

Shown below as SEQ ID NO:127 is the mature 1P5D10.2 heavy chain variable domain protein sequence, with CDRs underlined:

```
    1 EVQLVESGGG LVKPGGSLKL SCAASGFTFS DYYMYWVRQT
        PEKRLEWVAT
  51 ISDGGLYTYY QDSVKGRFTI SRDNAKNNLY LQMSSLKSED
        TAMYYCARED
101 DYDGDFYTMD YWGQGTSVTV SS
```

This is a murine subgroup III(D) heavy chain. The DNA sequence of the 1P5D10.2 heavy chain variable domain (from pYL468) is provided as SEQ ID NO:126.

Shown below as SEQ ID NO:132 is the 1P5D0.2 mature light chain variable domain protein sequence, with CDRs underlined:

```
    1 QIVLTQSPAI MSASPGEKVT ITCSASSSVS YMHWFQQKPG
        TSPKLWIYST
  51 SNLASGVPAR FSGSGSGTSY SLTISRMEAE DAATYYCQQR
        SSYPLTFGAG
101 TKLELK
```

This is a murine subgroup VI kappa light chain. The DNA sequence of the mature 1P5D10.2 light chain variable domain (from pYL471) is provided as SEQ ID NO:131.

### Example 6

### Anti-DR6 Antibodies Bind to Rat, Mouse and Human DR6

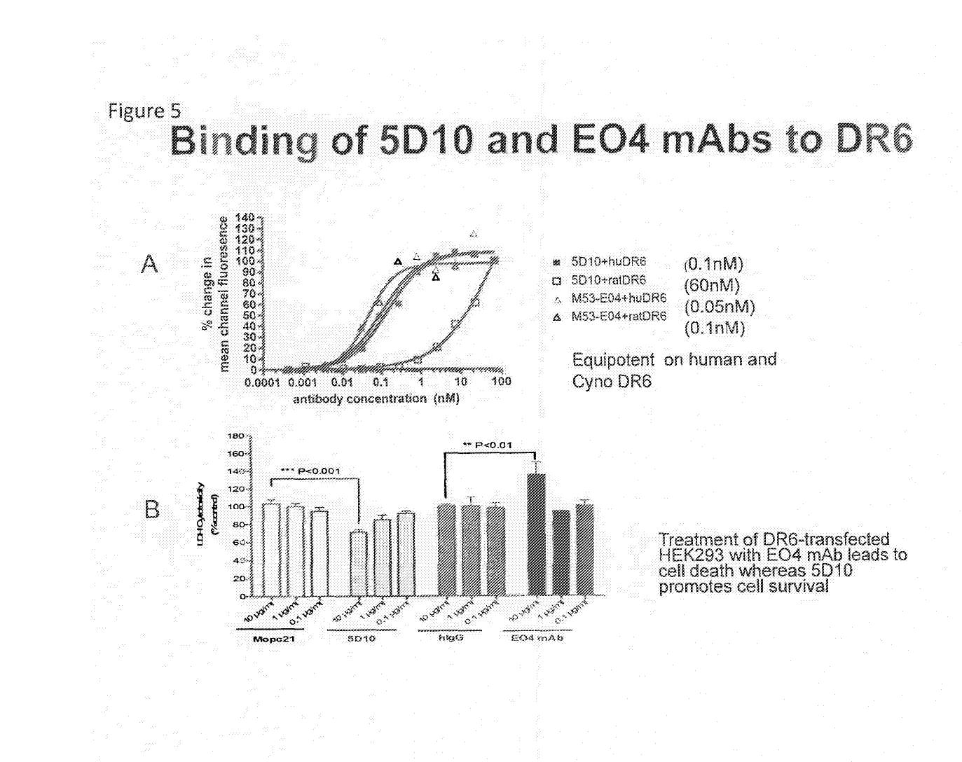

Six million HEK293 cells were transfected with 10 ug of plasmid DNA, which encoded full length human, rat, or mouse DR6. Three days after transfection, approximately 50,000 cells in 200 μL of PBS, 1% BSA, 0.1% NaN3 (FACS buffer) were analyzed. Cells were pelleted and resuspended in 150 μL of serial dilutions of anti-DR6 antibodies in FACS buffer. Samples were incubated for 1 hour on ice with occasional agitation and then washed three times. Bound DR6 antibody was visualized with PE-labeled goat F(ab)₂ anti-human Fab (for Dyax Fabs) or anti-mouse IgG specific antibody (for monoclonal antibodies) (Jackson Labs). The results, shown in FIG. 5A, demonstrate that the 5D10 and M53E04 antibodies each bind to human and rat DR6. Although both antibodies bind to DR6, the EO4 antibody leads to cell death, whereas 5D10 promotes cell survival. FIG. 5B.

### Example 7

### 5D10Y93A Binds Rat DR6 with Greater Affinity

The apparent Kd of 5D10 and M53-E04 for rat and human DR6 was estimated using transiently transfected HEK293 cells in a FACS direct binding assay. Approximately 50,000 cells in 150 uL of PBS, 1% BSA, 0.1% NaN3 (FACS buffer) were analyzed. Cells were pelleted and resuspended in 150 uL of serial dilutions of the antibodies in FACS buffer. Samples were incubated for 1 hr on ice with occasional agitation and then washed 3 times. Bound antibodies were visualized with PE-labeled goat F(ab)2 anti-human or anti-mouse Fab specific antibody (Jackson Labs).

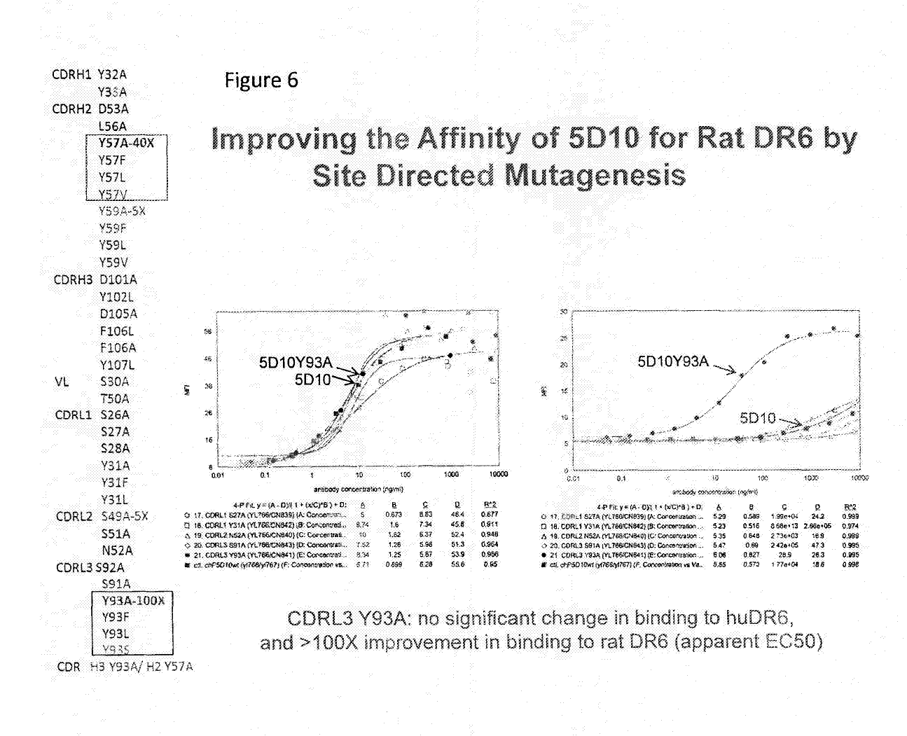

As shown in FIG. 5, the affinity of 5D10 to rat DR6 (EC50=60 nM) was much weaker than human DR6 (EC50=0.1 nM). In contrast, M53-E04 antibody showed similar binding affinity to human and rat DR6 (0.05 and 0.1 nM, respectively). The binding affinities of 5D10 or M53-E04 to cyno monkey DR6 were similar to human DR6. A single point mutation (Y93A) was introduced to 5D10 based on the co-crystal structure of DR6-5D10 (FIG. 6). The resulting antibody 5D10Y93A, showed significantly improved binding to rat DR6 compared to 5D10 (FIG. 6).

The present invention is not to be limited in scope by the specific embodiments described which are intended as single illustrations of individual aspects of the invention, and any compositions or methods which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims.

All publications and patent applications mentioned in this specification are herein incorporated by reference to the same extent as if each individual publication or patent application was specifically and individually indicated to be incorporated by reference.

It is to be appreciated that the Detailed Description section, and not the Summary and Abstract sections, is intended to be used to interpret the claims. The Summary and Abstract sections can set forth one or more but not all exemplary embodiments of the present invention as contemplated by the inventor(s), and thus, are not intended to limit the present invention and the appended claims in any way.

What is claimed is:

1. An isolated antibody or fragment thereof that can specifically bind to a DR6 polypeptide, wherein the VL of said antibody or fragment thereof comprises the amino acid sequence of SEQ ID NO:167.

2. An isolated antibody or fragment thereof that can specifically bind to a DR6 polypeptide, wherein the VH and VL of said antibody or fragment thereof comprise, respectively, the amino acid sequences of SEQ ID NO:127 and SEQ ID NO:167.

3. An isolated antibody or fragment thereof that can specifically bind to a DR6 polypeptide, wherein the VL of said antibody or fragment thereof comprises a Kabat light chain complementarity determining region-3 (VL-CDR3) amino acid sequence of SEQ ID NO:168.

4. The isolated antibody or fragment thereof of claim 3, wherein the VL of said antibody or fragment thereof comprises VL-CDR1, VL-CDR2, and VL-CDR3 amino acid sequences of: SEQ ID NOs: 133, 134, and 168.

5. The isolated antibody or fragment thereof of claim 3, wherein the VH of said antibody or fragment thereof comprises VH-CDR1, VH-CDR2, and VH-CDR3 amino acid sequences of SEQ ID NOs: 128, 129, and 130.

6. The isolated antibody or fragment thereof of claim 4, wherein the VH of said antibody or fragment thereof comprises VH-CDR1, VH-CDR2, and VH-CDR3 amino acid sequences of SEQ ID NOs: 128, 129, and 130.

7. The antibody or fragment thereof of any one of claims 3 to 6, wherein the VH framework regions are human, except for five or fewer amino acid substitutions.

8. The antibody or fragment thereof of any one of claims 3 to 6, wherein the VL framework regions are human, except for five or fewer amino acid substitutions.

9. The antibody or fragment thereof of any one of claims 1 to 8, which binds the Cys3-Cys4 domain of DR6.

10. The antibody or fragment thereof of any one of claims 1 to 9, wherein the heavy and light chain variable domains are murine in origin.

11. The antibody or fragment thereof of any one of claims 1 to 9, wherein the heavy and light chain variable domains are fully human in origin.

12. The antibody or fragment thereof of any one of claims 1 to 9, which is humanized.

13. The antibody or fragment thereof of any one of claims 1 to 9, which is chimeric.

14. The antibody or fragment thereof of any one of claims 1 to 9, which is primatized.

15. The antibody or fragment thereof of any one of claims 1 to 9, which is fully human.

16. The antibody or fragment thereof of any one of claims 1 to 15, which is an Fab fragment.

17. The antibody or fragment thereof of any one of claims 1 to 15, which is an Fab fragment.

18. The antibody or fragment thereof of any one of claims 1 to 15, which is an F(ab)2 fragment.

19. The antibody or fragment thereof of any one of claims 1 to 15, which is an Fv fragment.

20. The antibody or fragment thereof of any one of claims 1 to 15, which is a single chain antibody.

21. The antibody or fragment thereof of any one of claims 16 to 20, wherein said antibody or fragment thereof is conjugated to a polymer.

22. The antibody or fragment thereof of claim 21, wherein the polymer is a polyalkylene glycol.

23. The antibody or fragment thereof of claim 22, wherein the polyalkylene glycol is a polyethylene glycol (PEG).

24. The antibody or fragment thereof of any one of claims 1-18 and 20-23, which comprises a light chain constant region selected from the group consisting of a human kappa constant region and a human lambda constant region.

25. The antibody or fragment thereof of any one of claims 1-18 and 20-23, which comprises at a heavy chain constant region or fragment thereof.

26. The antibody or fragment thereof of claim 25, wherein said heavy chain constant region or fragment thereof is selected from the group consisting of human IgG4, IgG4 agly, IgG1 and IgG1 agly.

27. The antibody or fragment thereof of any one of claims 1 to 26 that inhibits binding of DR6 to p75.

28. The antibody or fragment thereof of any one of claims 1 to 27 that does not prevent binding of DR6 to APP.

29. A method of promoting survival of cells of the nervous system comprising contacting said cells with the antibody or fragment thereof of any one of claims 1-28.

30. The method of claim 29, wherein said cells are in a mammal and said contacting comprises administering a therapeutically effective amount of a DR6 antagonist to a mammal in need thereof.

31. A method of treating a condition associated with death of cells of the nervous system in a subject, the method comprising administering the antibody or fragment thereof of any one of claims 1-28.

32. The method of any one of claims 29-31, wherein the cells of the nervous system are cells of the central nervous system (CNS).

33. The method of claim 32, wherein the cells of the CNS are selected from the group consisting of cortical neurons, motor neurons, oligodendrocytes, microglia and astrocytes.

34. The method of any one of claims 29-31, wherein the cells of the nervous system are cells of the peripheral nervous system (PNS).

35. The method of claim 34, wherein the cells of the PNS are selected from the group consisting of dorsal root ganglion (DRG) neurons and schwann cells.

36. The method of any one of claims 29-31, wherein the cells of the nervous system are neurons.

37. The method of claim 36, wherein the neurons are cortical neurons, DRG neurons or motor neurons.

38. The method of any one of claims 29-31, wherein the cells of the nervous system are glial cells.

39. The method of claim 38, wherein the glial cells are selected from the group consisting of oligodendrocyte precursor cells (OPCs), schwann cells, astrocytes and microglial cells.

40. A method of promoting oligodendrocyte proliferation, differentiation or survival comprising contacting oligodendrocyte cells or oligodendrocyte precursor cells with the antibody or fragment thereof of any one of claims 1-28.

41. The method of claim 40, wherein said cells are in a mammal and said contacting comprises administering an effective amount of a DR6 antagonist to a mammal in need thereof.

42. A method of treating a condition associated with oligodendrocyte death or lack of differentiation comprising administering the antibody or fragment thereof of any one of claims 1-28.

43. A method of promoting myelination comprising contacting a mixture of neuronal cells and glial cells with the antibody or fragment thereof of any one of claims 1-28.

44. The method of claim 43, wherein the glial cells are oligodendrocyte cells or oligodendrocyte precursor cells.

45. The method of claim 44, wherein the glial cells are schwann cells.

46. The method of any one of claims 43-45, wherein said neuronal cells and said glial cells are in a mammal and said contacting comprises administering said antibody or fragment thereof to a mammal in need thereof.

47. A method of treating a condition associated with dysmyelination or demyelination comprising administering a therapeutically effective amount of the antibody or fragment thereof of any one of claims 1-28.

48. A method of inhibiting the binding of DR6 to p75 comprising contacting a DR6 polypeptide and/or p75 polypeptide with the antibody or fragment thereof of any one of claims 1-28 under conditions wherein binding of DR6 to p75 is inhibited.

49. The method of any one of claims 31-39, 42, 46, and 47, wherein said antibody or fragment thereof is administered by a route selected from the group consisting of topical administration, intraocular administration, intravitreal administration, parenteral administration, intrathecal administration, subdural administration, subcutaneous administration or via a capsule implant.

50. The method of any one of claims 30, 41, or 46 wherein said mammal has been diagnosed with or is suspected of having a condition associated death of cells of the central nervous system.

51. The method of claim 31 or 50, wherein said condition is selected from the group consisting of Alzheimer's disease, Parkinson's disease, Huntington's disease, motor neuron disease, (e.g. amyotrophic lateral sclerosis), multiple sclerosis, neuronal trauma and cerebral ischemia (e.g. stroke).

52. The method of claim 31, wherein the cells are schwann cells and the condition is neuropathic pain.

53. The method of claim 46, wherein said mammal has been diagnosed with or is suspected of having neuropathic pain.

54. The method of claim 47, wherein the condition is neuropathic pain.

55. The method of any one of claims 29 to 54, wherein said DR6 antagonist is used in combination with a p75 antagonist.

56. The method of claim 55, wherein the DR6 antagonist and the p75 antagonist are used simultaneously or sequentially.


SEQUENCE LISTING (170 sequences)

SEQ ID NO: 1 - DNA, 3660 bp, Homo sapiens
gccaccacgtgtgtccctgcgcccggtggccaccgactcagtccctcgccgaccagtctgggcagcggaggagggtggttggcagtggctggaagcttcgctatgggaagttgttcctttgctctctcgcgcccagtcctcctccctggttctcctcagccgctgtcggaggagagcacccggagacgcgggctgcagtcgcggcggcttctccccgcctgggcggccgcgccgctgggcaggtgctgagcgcccctagagcctcccttgccgcctccctcctctgcccggccgcagcagtgcacatggggtgttggaggtagatgggctcccggcccgggaggcggcggtggatgcggcgctgggcagaagcagccgccgattccagctgccccgcgcgccccgggcgcccctgcgagtccccggttcagccatggggacctctccgagcagcagcaccgccctcgcctcctgcagccgcatcgcccgccgagccacagccacgatgatcgcgggctcccttctcctgcttggattccttagcaccaccacagctcagccagaacagaaggcctcgaatctcattggcacataccgccatgttgaccgtgccaccggccaggtgctaacctgtgacaagtgtccagcaggaacctatgtctctgagcattgtaccaacacaagcctgcgcgtctgcagcagttgccctgtggggacctttaccaggcatgagaatggcatagagaaatgccatgactgtagtcagccatgcccatggccaatgattgagaaattaccttgtgctgccttgactgaccgagaatgcacttgcccacctggcatgttccagtctaacgctacctgtgccccccatacggtgtgtcctgtgggttggggtgtgcggaagaaagggacagagactgaggatgtgcggtgtaagcagtgtgctcggggtaccttctcagatgtgccttctagtgtgatgaaatgcaaagcatacacagactgtctgagtcagaacctggtggtgatcaagccggggaccaaggagacagacaacgtctgtggcacactcccgtccttctccagctccacctcaccttcccctggcacagccatctttccacgccctgagcacatggaaacccatgaagtcccttcctccacttatgttcccaaaggcatgaactcaacagaatccaactcttctgcctctgttagaccaaaggtactgagtagcatccaggaagggacagtccctgacaacacaagctcagcaagggggaaggaagacgtgaacaagaccctcccaaaccttcaggtagtcaaccaccagcaaggcccccaccacagacacatcctgaagctgctgccgtccatggaggccactgggggcgagaagtccagcacgcccatcaagggccccaagaggggacatcctagacagaacctacacaagcattttgacatcaatgagcatttgccctggatgattgtgcttttcctgctgctggtgcttgtggtgattgtggtgtgcagtatccggaaaagctcgaggactctgaaaaaggggccccggcaggatcccagtgccattgtggaaaaggcagggctgaagaaatccatgactccaacccagaaccgggagaaatggatctactactgcaatggccatggtatcgatatcctgaagcttgtagcagcccaagtgggaagccagtggaaagatatctatcagtttctttgcaatgccagtgagagggaggttgctgctttctccaatgggtacacagccgaccacgagcgggcctacgcagctctgcagcactggaccatccggggccccgaggccagcctcgcccagctaattagcgccctgcgccagcaccggagaaacgatgttgtggagaagattcgtgggctgatggaagacaccacccagctggaaactgacaaactagctctcccgatgagccccagcccgcttagcccgagccccatccccagccccaacgcgaaacttgagaattccgctctcctgacggtggagccttccccacaggacaagaacaagggcttcttcgtggatgagtcggagccccttctccgctgtgactctacatccagcggctcctccgcgctgagcaggaacggttcctttattaccaaagaaaagaaggacacagtgttgcggcaggtacgcctggacccctgtgacttgcagcctatctttgatgacatgctccactttctaaatcctgaggagctgcgggtgattgaagagattccccaggctgaggacaaactagaccggctattcgaaattattggagtcaagagccaggaagccagccagaccctcctggactctgtttatagccatcttcctgacctgctgtagaacatagggatactgcattctggaaattactcaatttagtggcagggtggttttttaattttcttctgtttctgatttttgttgtttggggtgtgtgtgtgtgtttgtgtgtgtgtgtgtgtgtgtgtgtgtgtgtgtgtttaacagagaatatggccagtgcttgagttctttctccttctctctctctcttttttttttaaataactcttctgggaagttggtttataagcctttgccaggtgtaactgttgtgaaatacccaccactaaagttttttaagttccatattttctccattttgccttcttatgtattttcaagattattctgtgcactttaaatttacttaacttaccataaatgcagtgtgacttttcccacacactggattgtgaggctcttaacttcttaaaagtataatggcatcttgtgaatcctataagcagtctttatgtctcttaacattcacacctactttttaaaaacaaatattattactatttttattattgtttgtcctttataaattttcttaaagattaagaaaatttaagaccccattgagttactgtaatgcaattcaactttgagttatcttttaaatatgtcttgtatagttcatattcatggctgaaacttgaccacactattgctgattgtatggttttcacctggacaccgtgtagaatgcttgattacttgtactcttcttatgctaatatgctctgggctggagaaatgaaatcctcaagccatcaggatttgctatttaagtggcttgacaactgggccaccaaagaacttgaacttcaccttttaggatttgagctgttctggaacacattgctgcactttggaaagtcaaaatcaagtgccagtggcgccctttccatagagaatttgcccagctttgctttaaaagatgtcttgttttttatatacacataatcaataggtccaatctgctctcaaggccttggtcctggtgggattccttcaccaattactttaattaaaaatggctgcaactgtaagaacccttgtctgatatatttgcaactatgctcccatttacaaatgtaccttctaatgctcagttgccaggttccaatgcaaaggtggcgtggactccctttgtgtgggtggggtttgtgggtagtggtgaaggaccgatatcagaaaaatgccttcaagtgtactaatttattaataaacattaggtgtttgttaaaaaaaaaaaaaaaaaaaaaaaaaaa

SEQ ID NO: 2 - protein, 655 aa, Homo sapiens
MGTSPSSSTALASCSRIARRATATMIAGSLLLLGFLSTTTAQPEQKASNLIGTYRHVDRATGQVLTCDKCPAGTYVSEHCTNTSLRVCSSCPVGTFTRHENGIEKCHDCSQPCPWPMIEKLPCAALTDRECTCPPGMFQSNATCAPHTVCPVGWGVRKKGTETEDVRCKQCARGTFSDVPSSVMKCKAYTDCLSQNLVVIKPGTKETDNVCGTLPSFSSSTSPSPGTAIFPRPEHMETHEVPSSTYVPKGMNSTESNSSASVRPKVLSSIQEGTVPDNTSSARGKEDVNKTLPNLQVVNHQQGPHHRHILKLLPSMEATGGEKSSTPIKGPKRGHPRQNLHKHFDINEHLPWMIVLFLLLVLVVIVVCSIRKSSRTLKKGPRQDPSAIVEKAGLKKSMTPTQNREKWIYYCNGHGIDILKLVAAQVGSQWKDIYQFLCNASEREVAAFSNGYTADHERAYAALQHWTIRGPEASLAQLISALRQHRRNDVVEKIRGLMEDTTQLETDKLALPMSPSPLSPSPIPSPNAKLENSALLTVEPSPQDKNKGFFVDESEPLLRCDSTSSGSSALSRNGSFITKEKKDTVLRQVRLDPCDLQPIFDDMLHFLNPEELRVIEEIPQAEDKLDRLFEIIGVKSQEASQTLLDSVYSHLPDLL

SEQ ID NO: 3 - DNA, 3626 bp, Mus musculus
gtgtccccgagctgagtggccatccgactcagtccctcgccggccggtctaggcagcggaggaggcgagtgcttgatagtggctggaagcttcgctatgggaagtcgctctttagcactgtcgcggctagccctgctctctggttctccgcagccgctgtcgttggagagcaccgggaggcgcgggttgcgagcgcgcctgcttctcaccgcccgggcgccagcgcccctgggcaggtgctgagcgcctttcggagcctcccctgctgcctccctcttccgcctgggtgcctggctgctgcagtgcacatgggctgctggaggtagatgggctcaccgcccgtgaggcggcggtggatgcggcgctgggcagaaacagccaccaattccagctgccgtggggccgagcgccccgggcgccgctgcgagccccgagctcggccatggggacccgggcaagcagcatcaccgccctcgcctcttgcagccgcaccgccggccaagtcggagccacgatggtcgccggctctcttctcctgcttggattcctcagcaccatcacagctcaaccagaacaaaagactctgagtctccctggcacctaccgccatgttgaccgtaccactggccaggtgctaacctgcgacaagtgcccagcaggaacgtacgtctccgagcactgtaccaacatgagcctgcgagtctgcagcagctgccccgcggggacctttaccaggcacgagaacggcatagagagatgccatgactgtagtcagccatgtccatggccgatgattgagagattaccttgtgctgccttgactgaccgagagtgcatctgcccacctggaatgtatcagtctaatggtacctgcgctccccatacagtgtgccccgtgggctggggtgtgcggaagaaagggacagagaatgaagatgtgcgctgtaagcagtgcgctcggggtaccttctctgacgtgccttccagtgtgatgaagtgtaaagctcacacggactgtctgggtcagaacctggaggtggtcaagccagggaccaaggagacagacaacgtctgtggcatgcgcctgttcttctccagcacaaacccaccttcctctggcacagttaccttttctcaccctgagcatatggaatcccacgatgtcccttcctccacctatgagccccaaggcatgaactcaacagattccaactctactgcctctgttagaactaaggtaccaagtggcatcgaggaagggacagtgcctgacaatacgagctcaaccagtgggaaggaaggcactaataggaccctgccaaacccaccacaagttacccaccagcaagccccccaccacagacacattctgaagctgctgccatcgtccatggaggccacgggtgagaagtccagcacagccatcaaggcccccaagaggggtcaccccagacagaacgctcacaagcatttcgacatcaacgagcacttgccttggatgatcgtcctcttccttctgctggtcctggtgctgatagtggtgtgcagtatccgaaagagctccaggactctcaaaaaggggccccggcaggatcccagcgccatagtggaaaaggcggggctgaagaagtccctgactcccacccagaaccgggagaaatggatctactaccgcaacggccatggtattgacatcttgaagcttgtagcagcccaggtgggaagccagtggaaggacatctatcagtttctttgcaacgccagcgagagggaggtggcggccttctccaatggatacactgcagatcatgaacgggcctacgcggctctgcagcactggaccatccgtggccctgaggccagccttgcccagctcattagcgccttgcgccagcaccgacgcaatgatgttgtggagaagattcgtgggctgatggaagacaccacgcagttggaaacagacaaactggctctccccatgagccccagtccgcttagcccgagccccatgcccagtcctaacgtgaaacttgagaattccactctcctgacagtggagccctcaccgctggacaagaacaagtgcttcttcgtggacgagtcagagccccttctgcgatgcgactccacatccagtggctcttcagcactgagcagaaacggctcctttattaccaaagaaaagaaggacacagtgttgcggcaggtccgcctggacccctgtgacttgcagcccatctttgatgacatgctgcatatcctgaaccccgaggagctgcgggtgattgaagagattccccaggctgaggacaaactggaccgcctcttcgagatcattggggtcaagagccaagaagccagccagaccctcttggactctgtgtacagtcatcttcctgacctattgtagaacacaggggcactgcattctgggaatcaacctactggcggggtgatttcatttcgtttctgacttttgtgttttggtgtgtatgtatgtgtttaacagagtgtatggccggtgagtttgggttctttctttctttctttctttctttctttctttctttctttctttctttctttctttctttctttcttccttcctgaaagtgaatgtataaagcctttacaatgtataactgttggaaaatgcccaccactaaatttttttttaagttccacatattctccatttttgccttcttatatatatcttcaacactattctgtgcactttaaaaacttaacataaacgcagtgtgacttctcccatatgctgggtcccgagactctcaacttcttaaaaacctaatggcatcttgtgactcctagaagtagacataagtctttcaaccttcacacctactctttctgttttaattattattgctatttgtcttattgtttgtgctttacaagcgttcttgaggacggagggaatctacgaccctgttgatgactgtaactctattcgactttgagttgtcttcttcatgtcttgttatatagttcatattcatggctgaaacttgaccatactccctagcgctgattgtatggttttcgtctggacaccgtacactgcctgataacttgtgcacctcttaacgctactatgctctgggctggagaatgaaatctttaagtcaccaggacttgctgtttcagtggcttgacacctgggccaccaaagaactcgatcttcatcttttagggacacctctgctgcaccttggaaagccaaccttaagtgccagtggcactttatgcccagctttgctttgaaagatatctttcttgttttttttatccttctctttctctcttttttttaaaaatacacatagtcaataggtccagtctgccctcaaggccttgctgggttttcttcatcatccaatcactttcattaaaaatggctgcagctgtaagaactcttgtctgataaattttcaactatgctctcatttatctacctgccctctgatgctcagtcgtcagactctaatgcaaaggtggacgtcggctgcctttgcgtgggcgggcttagtggtgaggaactgatatcagaaaaaaaatgccttcaagtatactaatttattaataaatattaggtgtttgtt

SEQ ID NO: 4 - protein, 655 aa, Mus musculus
MGTRASSITALASCSRTAGQVGATMVAGSLLLLGFLSTITAQPEQKTLSLPGTYRHVDRTTGQVLTCDKCPAGTYVSEHCTNMSLRVCSSCPAGTFTRHENGIERCHDCSQPCPWPMIERLPCAALTDRECICPPGMYQSNGTCAPHTVCPVGWGVRKKGTENEDVRCKQCARGTFSDVPSSVMKCKAHTDCLGQNLEVVKPGTKETDNVCGMRLFFSSTNPPSSGTVTFSHPEHMESHDVPSSTYEPQGMNSTDSNSTASVRTKVPSGIEEGTVPDNTSSTSGKEGTNRTLPNPPQVTHQQAPHHRHILKLLPSSMEATGEKSSTAIKAPKRGHPRQNAHKHFDINEHLPWMIVLFLLLVLVLIVVCSIRKSSRTLKKGPRQDPSAIVEKAGLKKSLTPTQNREKWIYYRNGHGIDILKLVAAQVGSQWKDIYQFLCNASEREVAAFSNGYTADHERAYAALQHWTIRGPEASLAQLISALRQHRRNDVVEKIRGLMEDTTQLETDKLALPMSPSPLSPSPMPSPNVKLENSTLLTVEPSPLDKNKCFFVDESEPLLRCDSTSSGSSALSRNGSFITKEKKDTVLRQVRLDPCDLQPIFDDMLHILNPEELRVIEEIPQAEDKLDRLFEIIGVKSQEASQTLLDSVYSHLPDLL

SEQ ID NO: 5 - protein, 8 aa, Artificial sequence
  Cyclic DR6 polypeptides
CLSPXXXC

SEQ ID NO: 6 - DNA, 357 bp, Artificial Sequence
  M50-H01 variable heavy chain
gaagttcaattgttagagtctggtggcggtcttgttcagcctggtggttctttacgtctttcttgcgctgcttccggattcactttctctgattaccctatgatgtgggttcgccaagctcctggtaaaggtttggagtgggtttctcgtatctctccttctggtggccatactatttatgctgactccgttaaaggtcgcttcactatctctagagacaactctaagaatactctctacttgcagatgaacagcttaagggctgaggacacggccgtgtattactgtgcgaaagattggcacggtggtggtgcttttgatatctggggccaagggacaatggtcaccgtctcaagc

SEQ ID NO: 7 - protein, 119 aa, Artificial sequence
  M50-H01 variable heavy chain
EVQLLESGGGLVQPGGSLRLSCAASGFTFSDYPMMWVRQAPGKGLEWVSRISPSGGHTIYADSVKGRFTISRDNSKNTLYLQMNSLRAEDTAVYYCAKDWHGGGAFDIWGQGTMVTVSS

SEQ ID NO: 8 - protein, 5 aa, Artificial Sequence
  M50-H01 VH CDR1
DYPMM

SEQ ID NO: 9 - protein, 17 aa, Artificial sequence
  M50-H01 VH CDR2
RISPSGGHTIYADSVKG

SEQ ID NO: 10 - protein, 10 aa, Artificial Sequence
  M50-H01  VH CDR3
DWHGGGAFDI

SEQ ID NO: 11 - DNA, 321 bp, Artificial Sequence
  M50-H01 variable light chain
gacatccagatgacccagtctccatcctctgtgtctgcatctgtaggagacagagtcaccatcacttgtcgggcgagtcagggtattagaagctggttagcctggtatcagcagaaaccagggaaagcccctaaactcctgatctatgatgcatccactttgcaaagtggggtcccatcaaggttcaggggcggtggctctgggacagatttcactctcactatcagcagcctgcagcctgaagattttgcaacttactactgtcaacagagttacagtaccccgctcactttcggcggagggaccaaggtggagatcaaa

SEQ ID NO: 12 - protein, 107 aa, Artificial sequence
  M50-H01 variable light chain
DIQMTQSPSSVSASVGDRVTITCRASQGIRSWLAWYQQKPGKAPKLLIYDASTLQSGVPSRFRGGGSGTDFTLTISSLQPEDFATYYCQQSYSTPLTFGGGTKVEIK

SEQ ID NO: 13 - protein, 11 aa, Artificial Sequence
  M50-H01 VL CDR1
RASQGIRSWLA

SEQ ID NO: 14 - protein, 7 aa, Artificial sequence
  M50-H01 VL CDR2
DASTLQS

SEQ ID NO: 15 - protein, 9 aa, Artificial Sequence
  M50-H01 VL CDR3
QQSYSTPLT

SEQ ID NO: 16 - DNA, 351 bp, Artificial sequence
  M51-H09 variable heavy chain
gaagttcaattgttagagtctggtggcggtcttgttcagcctggtggttctttacgtctttcttgcgctgcttccggattcactttctctccttactctatgacttgggttcgccaagctcctggtaaaggtttggagtgggtttcttctatcgttccttctggtggcaagacttggtatgctgactccgttaaaggtcgcttcactatctctagagacaactctaagaatactctctacttgcagatgaacagcttaagggctgaggacacggccgtgtattactgtgcgaggaccaatcttagctactttgactactggggccagggcaccctggtcaccgtctcaagc

SEQ ID NO: 17 - protein, 117 aa, Artificial sequence
  M51-H09 variable heavy chain
EVQLLESGGGLVQPGGSLRLSCAASGFTFSPYSMTWVRQAPGKGLEWVSSIVPSGGKTWYADSVKGRFTISRDNSKNTLYLQMNSLRAEDTAVYYCARTNLSYFDYWGQGTLVTVSS

SEQ ID NO: 18 - protein, 5 aa, Artificial Sequence
  M51-H09 VH CDR1
PYSMT

SEQ ID NO: 19 - protein, 17 aa, Artificial Sequence
  M51-H09 VH CDR2
SIVPSGGKTWYADSVKG

SEQ ID NO: 20 - protein, 8 aa, Artificial Sequence
  M51-H09 VH CDR3
TNLSYFDY

SEQ ID NO: 21 - DNA, 330 bp, Artificial Sequence
  M51-H09 variable light chain
cagagcgaattgactcagcctccctccgcgtccgggtctcctggacagtcagtcaccatctcctgcactggaaccagcaatgacgttggtagttataactatatctcctggttccaacagcacccaggcaaagcccccaaactcatgatttatgaggtcaataagcggccctcaggggtccctgatcgcttctctggctccaagtctgacaacacggcctccctgaccgtctctgggctccaggctgaagatgaggctgattattactgcacctcatatgcaggcagccacaagtgggtgttcggcggagggaccaagctgaccgtccta

SEQ ID NO: 22 - protein, 110 aa, Artificial Sequence
  M51-H09 variable light chain
QSELTQPPSASGSPGQSVTISCTGTSNDVGSYNYISWFQQHPGKAPKLMIYEVNKRPSGVPDRFSGSKSDNTASLTVSGLQAEDEADYYCTSYAGSHKWVFGGGTKLTVL

SEQ ID NO: 23 - protein, 14 aa, Artificial Sequence
  M51-H09 VL CDR1
TGTSNDVGSYNYIS

SEQ ID NO: 24 - protein, 7 aa, Artificial Sequence
  M51-H09 VL CDR2
EVNKRPS

SEQ ID NO: 25 - protein, 10 aa, Artificial Sequence
  M51-H09 VL CDR3
TSYAGSHKWV

SEQ ID NO: 26 - DNA, 345 bp, Artificial Sequence
  M53-E04 variable heavy chain
gaagttcaattgttagagtctggtggcggtcttgttcagcctggtggttctttacgtctttcttgcgctgcttccggattcactttctctaattacgagatgtcttgggttcgccaagctcctggtaaaggtttggagtgggtttcttctatctattcttctggtggcgagactctttatgctgactccgttaaaggtcgcttcactatctctagagacaactctaagaatactctctacttgcagatgaacagcttaagggctgaggacacggccgtgtattactgtgcgagagatggagcagctgactactggggccagggcaccctggtcaccgtctcaagc

SEQ ID NO: 27 - protein, 115 aa, Artificial Sequence
  M53-E04 variable heavy chain
EVQLLESGGGLVQPGGSLRLSCAASGFTFSNYEMSWVRQAPGKGLEWVSSIYSSGGETLYADSVKGRFTISRDNSKNTLYLQMNSLRAEDTAVYYCARDGAADYWGQGTLVTVSS

SEQ ID NO: 28 - protein, 5 aa, Artificial Sequence
  M53-E04 VH CDR1
NYEMS

SEQ ID NO: 29 - protein, 17 aa, Artificial Sequence
  M53-E04 VH CDR2
SIYSSGGETLYADSVKG

SEQ ID NO: 30 - protein, 6 aa, Artificial Sequence
  M53-E04 VH CDR3
DGAADY

SEQ ID NO: 31 - DNA, 330 bp, Artificial Sequence
  M53-E04 variable light chain
cagagcgaattgactcagccaccctcggcgtctgggacccccgggcagtgggtcaccatctcttgttctggaagcacctccaacatcggaaataattatgtatactggtaccagcagctcccaggaacggcccccaagctcctcatctatacgaataatcagcggccctcaggggtccctgaccgattctctggctccaagtctggcacgtccgcctccctggccatcagtgggctccggtccgaggatgaggctgattattactgtgcgacatgggatgacagtttgtatgctccggtattcggcggagggaccaaggtgaccgtccta

SEQ ID NO: 32 - protein, 110 aa, Artificial Sequence
  M53-E04 variable light chain
QSELTQPPSASGTPGQWVTISCSGSTSNIGNNYVYWYQQLPGTAPKLLIYTNNQRPSGVPDRFSGSKSGTSASLAISGLRSEDEADYYCATWDDSLYAPVFGGGTKVTVL

SEQ ID NO: 33 - protein, 13 aa, Artificial Sequence
  M53-E04 VL CDR1
SGSTSNIGNNYVY

SEQ ID NO: 34 - protein, 7 aa, Artificial Sequence
  M53-E04 VL CDR2
TNNQRPS

SEQ ID NO: 35 - protein, 11 aa, Artificial Sequence
  M53-E04 VL CDR3
ATWDDSLYAPV

SEQ ID NO: 36 - DNA, 351 bp, Artificial Sequence
  M53-F04 variable heavy chain
gaagttcaattgttagagtctggtggcggtcttgttcagcctggtggttctttacgtctttcttgcgctgcttccggattcactttctctgagtacactatgcattgggttcgccaagctcctggtaaaggtttggagtgggtttctgttatctcttcttctggtggccgtactgtttatgctgactccgttaaaggtcgcttcactatctctagagacaactctaagaatactctctacttgcagatgaacagcttaagggctgaggacacggccgtgtattactgtgcgagagatctgagtaactacatggactactggggccagggcaccctggtcaccgtctcaagc

SEQ ID NO: 37 - protein, 117 aa, Artificial Sequence
  M53-F04 variable heavy chain
EVQLLESGGGLVQPGGSLRLSCAASGFTFSEYTMHWVRQAPGKGLEWVSVISSSGGRTVYADSVKGRFTISRDNSKNTLYLQMNSLRAEDTAVYYCARDLSNYMDYWGQGTLVTVSS

SEQ ID NO: 38 - protein, 5 aa, Artificial Sequence
  M53-F04 VH CDR1
EYTMH

SEQ ID NO: 39 - protein, 17 aa, Artificial Sequence
  M53-F04 VH CDR2
VISSSGGRTVYADSVKG

SEQ ID NO: 40 - protein, 8 aa, Artificial Sequence
  M53-F04 VH CDR3
DLSNYMDY

SEQ ID NO: 41 - DNA, 318 bp, Artificial Sequence
  M53-F04 variable light chain
gacatccagatgacccagtctccagccaccctgtctttgtctccaggggaaagagccaccctctcctgcagggccagtcagactgttagcaactacttagcctggtaccaacagaaacctggccaggctcccaggctcctcatctatgatgcctccaacagggccactggcatcccagccaggttcagtggcagtgggtctgggacagacttcactctcaccatcagcagcctagagcctgaagattttgcagtttattactgtcagcagcgtagcaagtggctctctttcggcggagggaccaaggtggagatcaaa

SEQ ID NO: 42 - protein, 106 aa, Artificial Sequence
  M53-F04 variable light chain
DIQMTQSPATLSLSPGERATLSCRASQTVSNYLAWYQQKPGQAPRLLIYDASNRATGIPARFSGSGSGTDFTLTISSLEPEDFAVYYCQQRSKWLSFGGGTKVEIK

SEQ ID NO: 43 - protein, 11 aa, Artificial Sequence
  M53-F04 VL CDR1
RASQTVSNYLA

SEQ ID NO: 44 - protein, 7 aa, Artificial Sequence
  M53-F04 VL CDR2
DASNRAT

SEQ ID NO: 45 - protein, 8 aa, Artificial Sequence
  M53-F04 VL CDR3
QQRSKWLS

SEQ ID NO: 46 - DNA, 348 bp, Artificial Sequence
  M62-B02 variable heavy chain
gaagttcaattgttagagtctggtggcggtcttgttcagcctggtggttctttacgtctttcttgcgctgcttccggattcactttctctatgtacattatgaattgggttcgccaagctcctggtaaaggtttggagtgggtttcttctatcgtttcttctggtggctggactttttatgctgactccgttaaaggtcgcttcactatctctagagacaactctaagaatactctctacttgcagatgaacagcttaagggctgaggacacggccgtgtattactgtgcgaggggccactacggtatggacgtctggggccaagggaccacggtcaccgtctcaagc

SEQ ID NO: 47 - protein, 116 aa, Artificial Sequence
  M62-B02 variable heavy chain
EVQLLESGGGLVQPGGSLRLSCAASGFTFSMYIMNWVRQAPGKGLEWVSSIVSSGGWTFYADSVKGRFTISRDNSKNTLYLQMNSLRAEDTAVYYCARGHYGMDVWGQGTTVTVSS

SEQ ID NO: 48 - protein, 5 aa, Artificial Sequence
  M62-B02 VH CDR1
MYIMN

SEQ ID NO: 49 - protein, 17 aa, Artificial Sequence
  M62-B02 VH CDR2
SIVSSGGWTFYADSVKG

SEQ ID NO: 50 - protein, 7 aa, Artificial Sequence
  M62-B02 VH CDR3
GHYGMDV

SEQ ID NO: 51 - DNA, 339 bp, Artificial Sequence
  M62-B02 variable light chain
gacatccagatgacccagtctccagactccctggctgtgtctctgggcgagagggccaccatcaactgcaagtccagccggagtgttttatacagctccaacaataagaactacttagcttggtaccagcagaaaccaggacagcctcctaagctgctcatttactgggcttctacccgggaatccggggtccctgaccgattcagtggcagcgggtctgggacagatttcactctcaccatcagcagcctgcaggctgaagatgtggcagtttattactgtcagcaatattatagtactcctctcactttcggcggagggaccaaggtggagatcaaa

SEQ ID NO: 52 - protein, 113 aa, Artificial Sequence
  M62-B02 variable light chain
DIQMTQSPDSLAVSLGERATINCKSSRSVLYSSNNKNYLAWYQQKPGQPPKLLIYWASTRESGVPDRFSGSGSGTDFTLTISSLQAEDVAVYYCQQYYSTPLTFGGGTKVEIK

SEQ ID NO: 53 - protein, 17 aa, Artificial Sequence
  M62-B02 VL CDR1
KSSRSVLYSSNNKNYLA

SEQ ID NO: 54 - protein, 7 aa, Artificial Sequence
  M62-B02 VL CDR2
WASTRES

SEQ ID NO: 55 - protein, 9 aa, Artificial Sequence
  M62-B02 VL CDR3
QQYYSTPLT

SEQ ID NO: 56 - DNA, 363 bp, Artificial Sequence
  M63-E10 variable heavy chain
gaagttcaattgttagagtctggtggcggtcttgttcagcctggtggttctttacgtctttcttgcgctgcttccggattcactttctctcagtacaagatgtcttgggttcgccaagctcctggtaaaggtttggagtgggtttcttctatctcttcttctggtggcactaattatgctgactccgttaaaggtcgcttcactatctctagagacaactctaagaatactctctacttgcagatgaacagcttaagggctgaggacaccgccatgtattactgtgcgagacggaactacggtgacagaccttactggtacttcgatctctggggccgtggcaccctggtcaccgtctcaagc

SEQ ID NO: 57 - protein, 121 aa, Artificial Sequence
  M63-E10 variable heavy chain
EVQLLESGGGLVQPGGSLRLSCAASGFTFSQYKMSWVRQAPGKGLEWVSSISSSGGTNYADSVKGRFTISRDNSKNTLYLQMNSLRAEDTAMYYCARRNYGDRPYWYFDLWGRGTLVTVSS

SEQ ID NO: 58 - protein, 5 aa, Artificial Sequence
  M63-E10 VH CDR1
QYKMS

SEQ ID NO: 59 - protein, 16 aa, Artificial Sequence
  M63-E10 VH CDR2
SISSSGGTNYADSVKG

SEQ ID NO: 60 - protein, 13 aa, Artificial Sequence
  M63-E10 VH CDR3
RNYGDRPYWYFDL

SEQ ID NO: 61 - DNA, 321 bp, Artificial Sequence
  M63-E10 variable light chain
gacatccagatgacccagtctccttccaccctgtctgcatctgtaggagacagagtcaccatcacttgccgggccagtcagactattagtagatggttggcctggtatcagcacaaaccagggaaagcccctaaactcctgatctacaagacgtctaatttagaaagtggggtcccatcaaggttcagcggcagtggatctgggacagaattcactctcaccatcagtagcctgcagcctgatgattttgccacttattactgccaacagtataaaagttattcgatcaccttcggccctgggaccaaagtggatatcaaa

SEQ ID NO: 62 - protein, 107 aa, Artificial Sequence
  M63-E10 variable light chain
DIQMTQSPSTLSASVGDRVTITCRASQTISRWLAWYQHKPGKAPKLLIYKTSNLESGVPSRFSGSGSGTEFTLTISSLQPDDFATYYCQQYKSYSITFGPGTKVDIK

SEQ ID NO: 63 - protein, 11 aa, Artificial Sequence
  M63-E10 VL CDR1
RASQTISRWLA

SEQ ID NO: 64 - protein, 7 aa, Artificial Sequence
  M63-E10 VL CDR2
KTSNLES

SEQ ID NO: 65 - protein, 9 aa, Artificial Sequence
  M63-E10 VL CDR3
QQYKSYSIT

SEQ ID NO: 66 - DNA, 354 bp, Artificial Sequence
  M66-B03 variable heavy chain
gaagttcaattgttagagtctggtggcggtcttgttcagcctggtggttctttacgtctttcttgcgctgcttccggattcactttctctccttactggatgtcttgggttcgccaagctcctggtaaaggtttggagtgggtttctgttatctatccttctggtggccatacttattatgctgactccgttaaaggtcgcttcactatctctagagacaactctaagaatactctctacttgcagatgaacagcttaagggctgaggacacggccgtgtattactgtgcgaggccagggttcggggcggcttttgactactggggcccgggaaccctggtcaccgtctcaagc

SEQ ID NO: 67 - protein, 118 aa, Artificial Sequence
  M66-B03 variable heavy chain
EVQLLESGGGLVQPGGSLRLSCAASGFTFSPYWMSWVRQAPGKGLEWVSVIYPSGGHTYYADSVKGRFTISRDNSKNTLYLQMNSLRAEDTAVYYCARPGFGAAFDYWGPGTLVTVSS

SEQ ID NO: 68 - protein, 5 aa, Artificial Sequence
  M66-B03 VH CDR1
PYWMS

SEQ ID NO: 69 - protein, 17 aa, Artificial Sequence
  M66-B03 VH CDR2
VIYPSGGHTYYADSVKG

SEQ ID NO: 70 - protein, 9 aa, Artificial Sequence
  M66-B03 VH CDR3
PGFGAAFDY

SEQ ID NO: 71 - DNA, 321 bp, Artificial Sequence
  M66-B03 variable light chain
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttccccctcactttcggcggagggaccaaggtggagatcaaa

SEQ ID NO: 72 - protein, 107 aa, Artificial Sequence
  M66-B03 variable light chain
DIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIK

SEQ ID NO: 73 - protein, 11 aa, Artificial Sequence
  M66-B03 VL CDR1
RASQGISSWLA

SEQ ID NO: 74 - protein, 7 aa, Artificial Sequence
  M66-B03 VL CDR2
AASSLQS

SEQ ID NO: 75 - protein, 9 aa, Artificial Sequence
  M66-B03 VL CDR3
QQANSFPLT

SEQ ID NO: 76 - DNA, 384 bp, Artificial Sequence
  M67-G02 variable heavy chain
gaagttcaattgttagagtctggtggcggtcttgttcagcctggtggttctttacgtctttcttgcgctgcttccggattcactttctctcgttactctatgcagtgggttcgccaagctcctggtaaaggtttggagtgggtttctgttatctctccttctggtggctctactatgtatgctgactccgttaaaggtcgcttcactatctctagagacaactctaagaatactctctacttgcagatgaacagcttaagggctgaggacacggccgtgtattactgtgcgagagaatcatatagcagcagctggtattctgggtactactactactacggtatggacgtctggggccaagggaccacggtcaccgtctcaagc

SEQ ID NO: 77 - protein, 128 aa, Artificial Sequence
  M67-G02 variable heavy chain
EVQLLESGGGLVQPGGSLRLSCAASGFTFSRYSMQWVRQAPGKGLEWVSVISPSGGSTMYADSVKGRFTISRDNSKNTLYLQMNSLRAEDTAVYYCARESYSSSWYSGYYYYYGMDVWGQGTTVTVSS

SEQ ID NO: 78 - protein, 5 aa, Artificial Sequence
  M67-G02 VH CDR1
RYSMQ

SEQ ID NO: 79 - protein, 17 aa, Artificial Sequence
  M67-G02 VH CDR2
VISPSGGSTMYADSVKG

SEQ ID NO: 80 - protein, 19 aa, Artificial Sequence
  M67-G02 VH CDR3
ESYSSSWYSGYYYYYGMDV

SEQ ID NO: 81 - DNA, 321 bp, Artificial Sequence
  M67-G02 variable light chain
gacatccagatgacccagtctccagcctccctgtctgcatctgttggagacagagtcaccatcacttgccgggcaagtcagaccattgccggctatttaaattggtatcagcagacaccagggaaagcccctaatctcctgatctatgatgcatcccgtttgcaaagtggggtcccatcacggttcagtggcagtggatctgggacagatttcactctcagcatcagcggtctgcaacctgaagattttggaacttactactgtcaacagagttacaccatcccgctcactttcggcggagggaccaaggtggagatcaaa

SEQ ID NO: 82 - protein, 107 aa, Artificial Sequence
  M67-G02 variable light chain
DIQMTQSPASLSASVGDRVTITCRASQTIAGYLNWYQQTPGKAPNLLIYDASRLQSGVPSRFSGSGSGTDFTLSISGLQPEDFGTYYCQQSYTIPLTFGGGTKVEIK

SEQ ID NO: 83 - protein, 11 aa, Artificial Sequence
  M67-G02 VL CDR1
RASQTIAGYLN

SEQ ID NO: 84 - protein, 7 aa, Artificial Sequence
  M67-G02 VL CDR2
DASRLQS

SEQ ID NO: 85 - protein, 9 aa, Artificial Sequence
  M67-G02 VL CDR3
QQSYTIPLT

SEQ ID NO: 86 - DNA, 348 bp, Artificial Sequence
  M72-F03 variable heavy chain
gaagttcaattgttagagtctggtggcggtcttgttcagcctggtggttctttacgtctttcttgcgctgcttccggattcactttctctcagtacatgatgtcttgggttcgccaagctcctggtaaaggtttggagtgggtttctgttatctatccttctggtggctctactcattatgctgactccgttaaaggtcgcttcactatctctagagacaactctaagaatactctctacttgcagatgaacagcttaagggctgaggacacggccgtgtattactgtgcgagaggccactacggtatggacgtctggggccaggggaccacggtcaccgtctcaagc

SEQ ID NO: 87 - protein, 116 aa, Artificial Sequence
  M72-F03 variable heavy chain
EVQLLESGGGLVQPGGSLRLSCAASGFTFSQYMMSWVRQAPGKGLEWVSVIYPSGGSTHYADSVKGRFTISRDNSKNTLYLQMNSLRAEDTAVYYCARGHYGMDVWGQGTTVTVSS

SEQ ID NO: 88 - protein, 5 aa, Artificial Sequence
  M72-F03 VH CDR1
QYMMS

SEQ ID NO: 89 - protein, 18 aa, Artificial Sequence
  M72-F03 VH CDR2
VIYPSGGSTHYADSVKGR

SEQ ID NO: 90 - protein, 7 aa, Artificial Sequence
  M72-F03 VH CDR3
GHYGMDV

SEQ ID NO: 91 - DNA, 339 bp, Artificial Sequence
  M72-F03 variable light chain
gacatccagatgacccagtctccagactccctggctgtgtctctgggcgagagggccaccatcaactgcaagtccagccagagtgttttatacagctccaacaataagaactacttagcttggtatcagcataaagcaggacagcctcctaagctgctcattcactgggcatcttcccgggcatccggggtccctgaccgattcagtggcagcgggtctgggacagatttcactctcaccatcagcagcctgcaggctgaggatgtggccgtctattactgtcagcaattttacagtcttcctctcactttcggcggagggaccaaggtggagatcaga

SEQ ID NO: 92 - protein, 113 aa, Artificial Sequence
  M72-F03 variable light chain
DIQMTQSPDSLAVSLGERATINCKSSQSVLYSSNNKNYLAWYQHKAGQPPKLLIHWASSRASGVPDRFSGSGSGTDFTLTISSLQAEDVAVYYCQQFYSLPLTFGGGTKVEIR

SEQ ID NO: 93 - protein, 17 aa, Artificial Sequence
  M72-F03 VL CDR1
KSSQSVLYSSNNKNYLA

SEQ ID NO: 94 - protein, 7 aa, Artificial Sequence
  M72-F03 VL CDR2
WASSRAS

SEQ ID NO: 95 - protein, 9 aa, Artificial Sequence
  M72-F03 VL CDR3
QQFYSLPLT

SEQ ID NO: 96 - DNA, 360 bp, Artificial Sequence
  M73-C04 variable heavy chain
gaagttcaattgttagagtctggtggcggtcttgttcagcctggtggttctttacgtctttcttgcgctgcttccggattcactttctctccttacgctatggtttgggttcgccaagctcctggtaaaggtttggagtgggtttcttctatctctccttctggtggcggtactggttatgctgactccgttaaaggtcgcttcactatctctagagacaactctaagaatactctctacttgcagatgaacagcttaagggctgaggacacggccgtgtattactgtgcgagagaggggtggaactactactacggtatggacgtctggggccaagggaccacggtcaccgtctcaagc

SEQ ID NO: 97 - protein, 120 aa, Artificial Sequence
  M73-C04 variable heavy chain
EVQLLESGGGLVQPGGSLRLSCAASGFTFSPYAMVWVRQAPGKGLEWVSSISPSGGGTGYADSVKGRFTISRDNSKNTLYLQMNSLRAEDTAVYYCAREGWNYYYGMDVWGQGTTVTVSS

SEQ ID NO: 98 - protein, 5 aa, Artificial Sequence
  M73-C04 VH CDR1
PYAMV

SEQ ID NO: 99 - protein, 17 aa, Artificial Sequence
  M73-C04 VH CDR2
SISPSGGGTGYADSVKG

SEQ ID NO: 100 - protein, 11 aa, Artificial Sequence
  M73-C04 VH CDR3
EGWNYYYGMDV

SEQ ID NO: 101 - DNA, 321 bp, Artificial Sequence
  M73-C04 variable light chain
gacatccagatgacccagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctggcacagatttcactctcgccatcagcagcctgcagcctgaagattctgcaacttattactgtctacaagatttcatttacccgtacacttttggccaggggaccaagctggagatcaga

SEQ ID NO: 102 - protein, 107 aa, Artificial Sequence
  M73-C04 variable light chain
DIQMTQSPATLSLSPGERATLSCRASQSVSSYLAWYQQKPGQAPRLLIYDASNRATGIPARFSGSGSGTDFTLAISSLQPEDSATYYCLQDFIYPYTFGQGTKLEIR

SEQ ID NO: 103 - protein, 11 aa, Artificial Sequence
  M73-C04 VL CDR1
RASQSVSSYLA

SEQ ID NO: 104 - protein, 7 aa, Artificial Sequence
  M73-C04 VL CDR2
DASNRAT

SEQ ID NO: 105 - protein, 9 aa, Artificial Sequence
  M73-C04 VL CDR3
LQDFIYPYT

SEQ ID NO: 106 - DNA, 339 bp, Artificial Sequence
  1P1D6.3 variable heavy chain
caggttcagctgcagcagtctggaactgagctggcgaggcccggggcttcagtgaagctgtcctgcaaggcttctggctacaccttcactgactactatctaaactggatgaaacaggggactggacagggccttgagtggattggagagatttatcctggaggtgatcacacttactacaatgagaaattcaagggcaaggccacactgactgcagacaaatcctccaacacagccttcatgcagctcagcagcctgacatctgaggactctgcagtctatttctgtacaagaggggtgattaagtggggccaagggactctggtcactgtctcttta

SEQ ID NO: 107 - protein, 113 aa, Artificial Sequence
  1P1D6.3 variable heavy chain
QVQLQQSGTELARPGASVKLSCKASGYTFTDYYLNWMKQGTGQGLEWIGEIYPGGDHTYYNEKFKGKATLTADKSSNTAFMQLSSLTSEDSAVYFCTRGVIKWGQGTLVTVSL

SEQ ID NO: 108 - protein, 5 aa, Artificial Sequence
  1P1D6.3 VH CDR1
DYYLN

SEQ ID NO: 109 - protein, 17 aa, Artificial Sequence
  1P1D6.3 VH CDR2
EIYPGGDHTYYNEKFKG

SEQ ID NO: 110 - protein, 4 aa, Artificial Sequence
  1P1D6.3 VH CDR3
GVIK

SEQ ID NO: 111 - DNA, 321 bp, Artificial Sequence
  1P1D6.3 variable light chain
gacatcctgatgacccaatctccaccctccatgtctgtctctctgggagacacagtcagcatcacttgccatgcaagtcagggcattagcagtaatatagggtggttgcagcagaaaccagggaaatcatttaagggcctgatctatcatggatccaccttggaggatggagttccatcaagattcagtggcagtggatctggagcagaattttctctcaccatcagcagcctggaatctgaagattttgcagactattactgtgttcaatatgctcagtttccttacacgttcggaggggggaccaagctagaaataaaa

SEQ ID NO: 112 - protein, 107 aa, Artificial Sequence
  1P1D6.3 variable light chain
DILMTQSPPSMSVSLGDTVSITCHASQGISSNIGWLQQKPGKSFKGLIYHGSTLEDGVPSRFSGSGSGAEFSLTISSLESEDFADYYCVQYAQFPYTFGGGTKLEIK

SEQ ID NO: 113 - protein, 11 aa, Artificial Sequence
  1P1D6.3 VL CDR1
HASQGISSNIG

SEQ ID NO: 114 - protein, 7 aa, Artificial Sequence
  1P1D6.3 VL CDR2
HGSTLED

SEQ ID NO: 115 - protein, 9 aa, Artificial Sequence
  1P1D6.3 VL CDR3
VQYAQFPYT

SEQ ID NO: 116 - DNA, 339 bp, Artificial Sequence
  1P2F2.1 variable heavy chain
caggttcagctgcagcagtctggacctgaggtggcgaggcccggggcttcagtgaagctgtcctgcaaggcttctggctacaccttcactgactactatttaaactgggtgaagcagaggactggacagggccttgagtggattggagagatttatcctggaaataatcatacttactacaatgagaagttcaagggcaaggccacactgactgcagacaattcctccagcacagcctacttgcagttcagcagcctgacatctgaggactctgctgtctatttctgtacaagaggggtgattaagtggggccaagggactctggtcactgtctctgta

SEQ ID NO: 117 - protein, 113 aa, Artificial Sequence
  1P2F2.1 variable heavy chain
QVQLQQSGPEVARPGASVKLSCKASGYTFTDYYLNWVKQRTGQGLEWIGEIYPGNNHTYYNEKFKGKATLTADNSSSTAYLQFSSLTSEDSAVYFCTRGVIKWGQGTLVTVSV

SEQ ID NO: 118 - protein, 5 aa, Artificial Sequence
  1P2F2.1 VH CDR1
DYYLN

SEQ ID NO: 119 - protein, 17 aa, Artificial Sequence
  1P2F2.1 VH CDR2
EIYPGNNHTYYNEKFKG

SEQ ID NO: 120 - protein, 4 aa, Artificial Sequence
  1P2F2.1 VH CDR3
GVIK

SEQ ID NO: 121 - DNA, 321 bp, Artificial Sequence
  1P2F2.1 variable light chain
gacatcctgatgacccaatctccatcctccatgtctgtatctctgggagacacagtcagcatcacttgccatgccagtcagggcattaggaatagtatagggtggttgcagcagaaaccagggaaatcatttaagggcctgatctatcatgcaaccaccttggaagatggagttccatcaaggttcactggcagtggatctggagcagatttttctctcaccatcagcagcctggaatctgaagattttgcagactattactgtgtacagtacgctcagtttccttacacgttcggaggggggaccaagctggaaataaaa

SEQ ID NO: 122 - protein, 107 aa, Artificial Sequence
  1P2F2.1 variable light chain
DILMTQSPSSMSVSLGDTVSITCHASQGIRNSIGWLQQKPGKSFKGLIYHATTLEDGVPSRFTGSGSGADFSLTISSLESEDFADYYCVQYAQFPYTFGGGTKLEIK

SEQ ID NO: 123 - protein, 11 aa, Artificial Sequence
  1P2F2.1 VL CDR1
HASQGIRNSIG

SEQ ID NO: 124 - protein, 7 aa, Artificial Sequence
  1P2F2.1 VL CDR2
HATTLED

SEQ ID NO: 125 - protein, 9 aa, Artificial Sequence
  1P2F2.1 VL CDR3
VQYAQFPYT

SEQ ID NO: 126 - DNA, 366 bp, Artificial Sequence
  1P5D10.2 variable heavy chain
gaagtgcagctggtggagtcggggggaggcttagtgaagcctggagggtccctgaaactctcctgtgcagcctctggattcactttcagtgactattacatgtattgggttcgccagactccggaaaagaggctggagtgggtcgcaaccattagtgatggtggtctttacacctactatcaagacagtgtgaaggggcgattcaccatctccagagacaatgccaagaacaacctgtacctgcaaatgagcagtctgaagtctgaggacacagccatgtattactgtgcaagagaagatgattacgacggggatttctatactatggactactggggtcaaggaacctcagtcaccgtctcctca

SEQ ID NO: 127 - protein, 122 aa, Artificial Sequence
  1P5D10.2 variable heavy chain
EVQLVESGGGLVKPGGSLKLSCAASGFTFSDYYMYWVRQTPEKRLEWVATISDGGLYTYYQDSVKGRFTISRDNAKNNLYLQMSSLKSEDTAMYYCAREDDYDGDFYTMDYWGQGTSVTVSS

SEQ ID NO: 128 - protein, 5 aa, Artificial Sequence
  1P5D10.2 VH CDR1
DYYMY

SEQ ID NO: 129 - protein, 17 aa, Artificial Sequence
  1P5D10.2 VH CDR2
TISDGGLYTYYQDSVKG

SEQ ID NO: 130 - protein, 13 aa, Artificial Sequence
  1P5D10.2 VH CDR3
EDDYDGDFYTMDY

SEQ ID NO: 131 - DNA, 318 bp, Artificial Sequence
  1P5D10.2 variable light chain
caaattgttctcacccagtctccagcaatcatgtctgcatctccaggggagaaggtcaccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttacccactcacgttcggtgctgggaccaagctggagctgaaa

SEQ ID NO: 132 - protein, 106 aa, Artificial Sequence
  1P5D10.2 variable light chain
QIVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELK

SEQ ID NO: 133 - protein, 10 aa, Artificial Sequence
  1P5D10.2 VL CDR1
SASSSVSYMH

SEQ ID NO: 134 - protein, 7 aa, Artificial Sequence
  1P5D10.2 VL CDR2
STSNLAS

SEQ ID NO: 135 - protein, 9 aa, Artificial Sequence
  1P5D10.2 VL CDR3
QQRSSYPLT

SEQ ID NO: 136 - protein, 15 aa, Artificial Sequence
  flexible sequence linker
GGGGSGGGGSGGGGS

SEQ ID NO: 137 - protein, 12 aa, Artificial Sequence
  flexible linker sequence
GGGGGSGGGGGS

SEQ ID NO: 138 - protein, 15 aa, Artificial Sequence
  flexible linker sequence
ESGRSGGGGSGGGGS

SEQ ID NO: 139 - protein, 14 aa, Artificial Sequence
  flexible linker sequence
EGKSSGSGSESKST

SEQ ID NO: 140 - protein, 15 aa, Artificial Sequence
  flexible linker sequence
EGKSSGSGSESKSTQ

SEQ ID NO: 141 - protein, 14 aa, Artificial Sequence
  flexible linker sequence
EGKSSGSGSESKVD

SEQ ID NO: 142 - protein, 14 aa, Artificial Sequence
  flexible linker sequence
GSTSGSGKSSEGKG

SEQ ID NO: 143 - protein, 18 aa, Artificial Sequence
  flexible linker sequence
KESGSVSSEQLAQFRSLD

SEQ ID NO: 144 - protein, 16 aa, Artificial Sequence
  flexible linker sequence
ESGSVSSEELAFRSLD

SEQ ID NO: 145 - protein, 8 aa, Artificial Sequence
  flag peptide
DYKDDDDK

SEQ ID NO: 146 - protein, 8 aa, Artificial Sequence
  flag peptide
DYKDEDDK

SEQ ID NO: 147 - protein, 9 aa, Artificial Sequence
  strep epitope
AWRHPQFGG

SEQ ID NO: 148 - protein, 11 aa, Artificial Sequence
  VSV-G epitope
YTDIEMNRLGK

SEQ ID NO: 149 - protein, 6 aa, Artificial Sequence
  Poly-His tag
HHHHHH

SEQ ID NO: 150 - protein, 13 aa, Artificial Sequence
  HA epitope
YPYDVPDYAIEGR

SEQ ID NO: 151 - protein, 11 aa, Artificial Sequence
  C-MYC epitope
EQKLLSEEDLN

SEQ ID NO: 152 - DNA, 40 bp, Artificial Sequence
  DR6 anti-sense probe
taatacgactcactataggggctggtgggtaagttgtggt

SEQ ID NO: 153 - DNA, 40 bp, Artificial Sequence
  DR6 sense probe
atttaggtgacactatagaactcgcggtaccttctctgac

SEQ ID NO: 154 - DNA, 1998 bp, Artificial Sequence
  human DR6 with Myc tag
atggggacctctccgagcagcagcaccgccctcgcctcctgcagccgcatcgcccgccgagccacagccacgatgatcgcgggctcccttctcctgcttggattccttagcaccaccacagctgagcagaagctgatctcagaggaggacctgcagccagaacagaaggcctcgaatctcattggcacataccgccatgttgaccgtgccaccggccaggtgctaacctgtgacaagtgtccagcaggaacctatgtctctgagcattgtaccaacacaagcctgcgcgtctgcagcagttgccctgtggggacctttaccaggcatgagaatggcatagagaaatgccatgactgtagtcagccatgcccatggccaatgattgagaaattaccttgtgctgccttgactgaccgagaatgcacttgcccacctggcatgttccagtctaacgctacctgtgccccccatacggtgtgtcctgtgggttggggtgtgcggaagaaagggacagagactgaggatgtgcggtgtaagcagtgtgctcggggtaccttctcagatgtgccttctagtgtgatgaaatgcaaagcatacacagactgtctgagtcagaacctggtggtgatcaagccggggaccaaggagacagacaacgtctgtggcacactcccgtccttctccagctccacctcaccttcccctggcacagccatctttccacgccctgagcacatggaaacccatgaagtcccttcctccacttatgttcccaaaggcatgaactcaacagaatccaactcttctgcctctgttagaccaaaggtactgagtagcatccaggaagggacagtccctgacaacacaagctcagcaagggggaaggaagacgtgaacaagaccctcccaaaccttcaggtagtcaaccaccagcaaggcccccaccacagacacatcctgaagctgctgccgtccatggaggccactgggggcgagaagtccagcacgcccatcaagggccccaagaggggacatcctagacagaacctacacaagcattttgacatcaatgagcatttgccctggatgattgtgcttttcctgctgctggtgcttgtggtgattgtggtgtgcagtatccggaaaagctcgaggactctgaaaaaggggccccggcaggatcccagtgccattgtggaaaaggcagggctgaagaaatccatgactccaacccagaaccgggagaaatggatctactactgcaatggccatggtatcgatatcctgaagcttgtagcagcccaagtgggaagccagtggaaagatatctatcagtttctttgcaatgccagtgagagggaggttgctgctttctccaatgggtacacagccgaccacgagcgggcctacgcagctctgcagcactggaccatccggggccccgaggccagcctcgcccagctaattagcgccctgcgccagcaccggagaaacgatgttgtggagaagattcgtgggctgatggaagacaccacccagctggaaactgacaaactagctctcccgatgagccccagcccgcttagcccgagccccatccccagccccaacgcgaaacttgagaattccgctctcctgacggtggagccttccccacaggacaagaacaagggcttcttcgtggatgagtcggagccccttctccgctgtgactctacatccagcggctcctccgcgctgagcaggaacggttcctttattaccaaagaaaagaaggacacagtgttgcggcaggtacgcctggacccctgtgacttgcagcctatctttgatgacatgctccactttctaaatcctgaggagctgcgggtgattgaagagattccccaggctgaggacaaactagaccggctattcgaaattattggagtcaagagccaggaagccagccagaccctcctggactctgtttatagccatcttcctgacctgctgtag

SEQ ID NO: 155 - protein, 665 aa, Artificial Sequence
  human DR6 with Myc tag
MGTSPSSSTALASCSRIARRATATMIAGSLLLLGFLSTTTAEQKLISEEDLQPEQKASNLIGTYRHVDRATGQVLTCDKCPAGTYVSEHCTNTSLRVCSSCPVGTFTRHENGIEKCHDCSQPCPWPMIEKLPCAALTDRECTCPPGMFQSNATCAPHTVCPVGWGVRKKGTETEDVRCKQCARGTFSDVPSSVMKCKAYTDCLSQNLVVIKPGTKETDNVCGTLPSFSSSTSPSPGTAIFPRPEHMETHEVPSSTYVPKGMNSTESNSSASVRPKVLSSIQEGTVPDNTSSARGKEDVNKTLPNLQVVNHQQGPHHRHILKLLPSMEATGGEKSSTPIKGPKRGHPRQNLHKHFDINEHLPWMIVLFLLLVLVVIVVCSIRKSSRTLKKGPRQDPSAIVEKAGLKKSMTPTQNREKWIYYCNGHGIDILKLVAAQVGSQWKDIYQFLCNASEREVAAFSNGYTADHERAYAALQHWTIRGPEASLAQLISALRQHRRNDVVEKIRGLMEDTTQLETDKLALPMSPSPLSPSPIPSPNAKLENSALLTVEPSPQDKNKGFFVDESEPLLRCDSTSSGSSALSRNGSFITKEKKDTVLRQVRLDPCDLQPIFDDMLHFLNPEELRVIEEIPQAEDKLDRLFEIIGVKSQEASQTLLDSVYSHLPDLL

SEQ ID NO: 156 - DNA, 1143 bp, Artificial Sequence
  dominant negative DR6 with Myc tag
atggggacctctccgagcagcagcaccgccctcgcctcctgcagccgcatcgcccgccgagccacagccacgatgatcgcgggctcccttctcctgcttggattccttagcaccaccacagctgagcagaagctgatctcagaggaggacctgcagccagaacagaaggcctcgaatctcattggcacataccgccatgttgaccgtgccaccggccaggtgctaacctgtgacaagtgtccagcaggaacctatgtctctgagcattgtaccaacacaagcctgcgcgtctgcagcagttgccctgtggggacctttaccaggcatgagaatggcatagagaaatgccatgactgtagtcagccatgcccatggccaatgattgagaaattaccttgtgctgccttgactgaccgagaatgcacttgcccacctggcatgttccagtctaacgctacctgtgccccccatacggtgtgtcctgtgggttggggtgtgcggaagaaagggacagagactgaggatgtgcggtgtaagcagtgtgctcggggtaccttctcagatgtgccttctagtgtgatgaaatgcaaagcatacacagactgtctgagtcagaacctggtggtgatcaagccggggaccaaggagacagacaacgtctgtggcacactcccgtccttctccagctccacctcaccttcccctggcacagccatctttccacgccctgagcacatggaaacccatgaagtcccttcctccacttatgttcccaaaggcatgaactcaacagaatccaactcttctgcctctgttagaccaaaggtactgagtagcatccaggaagggacagtccctgacaacacaagctcagcaagggggaaggaagacgtgaacaagaccctcccaaaccttcaggtagtcaaccaccagcaaggcccccaccacagacacatcctgaagctgctgccgtccatggaggccactgggggcgagaagtccagcacgcccatcaagggccccaagaggggacatcctagacagaacctacacaagcattttgacatcaatgagcatttgccctggatgattgtgcttttcctgctgctggtgcttgtggtgattgtggtgtgcagtatctag

SEQ ID NO: 157 - protein, 380 aa, Artificial Sequence
  dominant negative DR6 with Myc tag
MGTSPSSSTALASCSRIARRATATMIAGSLLLLGFLSTTTAEQKLISEEDLQPEQKASNLIGTYRHVDRATGQVLTCDKCPAGTYVSEHCTNTSLRVCSSCPVGTFTRHENGIEKCHDCSQPCPWPMIEKLPCAALTDRECTCPPGMFQSNATCAPHTVCPVGWGVRKKGTETEDVRCKQCARGTFSDVPSSVMKCKAYTDCLSQNLVVIKPGTKETDNVCGTLPSFSSSTSPSPGTAIFPRPEHMETHEVPSSTYVPKGMNSTESNSSASVRPKVLSSIQEGTVPDNTSSARGKEDVNKTLPNLQVVNHQQGPHHRHILKLLPSMEATGGEKSSTPIKGPKRGHPRQNLHKHFDINEHLPWMIVLFLLLVLVVIVVCSI

SEQ ID NO: 158 - DNA, 1731 bp, Artificial Sequence
  soluble DR6 Fc
atggggacctctccgagcagcagcaccgccctcgcctcctgcagccgcatcgcccgccgagccacagccacgatgatcgcgggctcccttctcctgcttggattccttagcaccaccacagctcagccagaacagaaggcctcgaatctcattggcacataccgccatgttgaccgtgccaccggccaggtgctaacctgtgacaagtgtccagcaggaacctatgtctctgagcattgtaccaacacaagcctgcgcgtctgcagcagttgccctgtggggacctttaccaggcatgagaatggcatagagaaatgccatgactgtagtcagccatgcccatggccaatgattgagaaattaccttgtgctgccttgactgaccgagaatgcacttgcccacctggcatgttccagtctaacgctacctgtgccccccatacggtgtgtcctgtgggttggggtgtgcggaagaaagggacagagactgaggatgtgcggtgtaagcagtgtgctcggggtaccttctcagatgtgccttctagtgtgatgaaatgcaaagcatacacagactgtctgagtcagaacctggtggtgatcaagccggggaccaaggagacagacaacgtctgtggcacactcccgtccttctccagctccacctcaccttcccctggcacagccatctttccacgccctgagcacatggaaacccatgaagtcccttcctccacttatgttcccaaaggcatgaactcaacagaatccaactcttctgcctctgttagaccaaaggtactgagtagcatccaggaagggacagtccctgacaacacaagctcagcaagggggaaggaagacgtgaacaagaccctcccaaaccttcaggtagtcaaccaccagcaaggcccccaccacagacacatcctgaagctgctgccgtccatggaggccactgggggcgagaagtccagcacgcccatcaagggccccaagaggggacatcctagacagaacctacacaagcattttgacatcaatgagcatgtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggtga

SEQ ID NO: 159 - protein, 576 aa, Artificial Sequence
  soluble DR6 Fc
MGTSPSSSTALASCSRIARRATATMIAGSLLLLGFLSTTTAQPEQKASNLIGTYRHVDRATGQVLTCDKCPAGTYVSEHCTNTSLRVCSSCPVGTFTRHENGIEKCHDCSQPCPWPMIEKLPCAALTDRECTCPPGMFQSNATCAPHTVCPVGWGVRKKGTETEDVRCKQCARGTFSDVPSSVMKCKAYTDCLSQNLVVIKPGTKETDNVCGTLPSFSSSTSPSPGTAIFPRPEHMETHEVPSSTYVPKGMNSTESNSSASVRPKVLSSIQEGTVPDNTSSARGKEDVNKTLPNLQVVNHQQGPHHRHILKLLPSMEATGGEKSSTPIKGPKRGHPRQNLHKHFDINEHVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 160 - RNA, 19 nt, Artificial Sequence
  DR6 siRNA
agaaacggcuccuuuauua

SEQ ID NO: 161 - RNA, 19 nt, Artificial Sequence
  DR6 siRNA
ggaaggacaucuaucaguu

SEQ ID NO: 162 - RNA, 19 nt, Artificial Sequence
  DR6 siRNA
ggccgaugauugagagauu

SEQ ID NO: 163 - RNA, 19 nt, Artificial Sequence
  DR6 siRNA
gcaguuggaaacagacaaa

SEQ ID NO: 164 - RNA, 21 nt, Artificial Sequence
  control siRNA
ggugacaugaucgacagccau

SEQ ID NO: 165 - protein, 427 aa, Homo sapiens
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 166 - DNA, 3421 bp, Homo sapiens
agagcgagccgagccgcggccagctccggcgggcagggggggcgctggagcgcagcgcagcgcagccccatcagtccgcaaagcggaccgagctggaagtcgagcgctgccgcgggaggcgggcgatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagctgcaagcagaacaagcaaggagccaacagccggccagtgaaccagacgcccccaccagagggagaaaaactccacagcgacagtggcatctccgtggacagccagagcctgcatgaccagcagccccacacgcagacagcctcgggccaggccctcaagggtgacggaggcctctacagcagcctgcccccagccaagcgggaggaggtggagaagcttctcaacggctctgcgggggacacctggcggcacctggcgggcgagctgggctaccagcccgagcacatagactcctttacccatgaggcctgccccgttcgcgccctgcttgcaagctgggccacccaggacagcgccacactggacgccctcctggccgccctgcgccgcatccagcgagccgacctcgtggagagtctgtgcagtgagtccactgccacatccccggtgtgagcccaaccggggagcccccgccccgccccacattccgacaaccgatgctccagccaacccctgtggagcccgcacccccaccctttggggggggcccgcctggcagaactgagctcctctgggcaggacctcagagtccaggccccaaaaccacagccctgtcagtgcagcccgtgtggccccttcacttctgaccacacttcctgtccagagagagaagtgcccctgctgcctccccaaccctgcccctgccccgtcaccatctcaggccacctgcccccttctcccacactgctaggtgggccagcccctcccaccacagcaggtgtcatatatggggggccaacaccagggatggtactagggggaagtgacaaggccccagagactcagagggaggaatcgaggaaccagagccatggactctacactgtgaacttggggaacaagggtggcatcccagtggcctcaaccctccctcagcccctcttgccccccaccccagcctaagatgaagaggatcggaggcttgtcagagctgggaggggttttcgaagctcagcccacccccctcattttggatataggtcagtgaggcccagggagaggccatgattcgcccaaagccagacagcaacggggaggccaagtgcaggctggcaccgccttctctaaatgaggggcctcaggtttgcctgagggcgaggggagggtggcaggtgaccttctgggaaatggcttgaagccaagtcagctttgccttccacgctgtctccagacccccaccccttccccactgcctgcccacccgtggagatgggatgcttgcctagggcctggtccatgatggagtcaggtttggggttcgtggaaagggtgctgcttccctctgcctgtccctctcaggcatgcctgtgtgacatcagtggcatggctccagtctgctgccctccatcccgacatggacccggagctaacactggcccctagaatcagcctaggggtcagggaccaaggacccctcaccttgcaacacacagacacacgcacacacacacacaggaggagaaatctcacttttctccatgagttttttctcttgggctgagactggatactgcccggggcagctgccagagaagcatcggagggaattgaggtctgctcggccgtcttcactcgcccccgggtttggcgggccaaggactgccgaccgaggctggagctggcgtctgtcttcaagggcttacacgtggaggaatgctcccccatcctccccttccctgcaaacatggggttggctgggcccagaaggttgtgatgaagaaaagtgggccagtgtgggaatgcggcaagaaggaattgacttcgactgtgacctgtggggatttctcccagctctagacaaccctgcaaaggactgttttttcctgagcttggccagaagggggccatgaggcctcagtggactttccaccccctccctggcctgttctgttttgcctgaagttggagtgagtgtggctcccctctatttagcatgacaagccccaggcaggctgtgcgctgacaaccaccgctccccagcccagggttcccccagccctgtggaagggactaggagcactgtagtaaatggcaattctttgacctcaacctgtgatgaggggaggaaactcacctgctggcccctcacctgggcacctggggagtgggacagagtctgggtgtatttattttcctccccagcaggtggggagggggtttgggggcttgcaagtatgttttagcatgtgtttggttctggggcccctttttactccccttgagctgagatggaacccttttggcccccgagctgggggccatgagctccagacccccagcaaccctcctatcacctcccctccttgcctcctgtgtaatcatttcttgggccctcctgaaacttacacacaaaacgttaagtgatgaacattaaatagcaaagaaagaaaaataaaaaaaaaaaa

SEQ ID NO: 167 - protein, 106 aa, Artificial Sequence
  1P5D10.2 Y93A VL
QIVLTQSPAIMSASPGEKVTITCSASSSVSYMHWFQQKPGTSPKLWIYSTSNLASGVPARFSGSGSGTSYSLTISRMEAEDAATYYCQQRSSAPLTFGAGTKLELK

SEQ ID NO: 168 - protein, 9 aa, Artificial Sequence
  1P5D10.2 Y93A VL CDR3
QQRSSAPLT

SEQ ID NO: 169 - DNA, 4350 bp, Rattus norvegicus
acgtgtgtccccgagctgagtggccatccgactcagtccctcaccggccggtctgggcagcggaggagacgagtggttggtagtggctggaagcttcgctatgggaagtcgctctttagcactgtcgccgctcgccctgctccctggttctccgcagccgctgtcgctggagagcaccgggaggcgcgggttgcaagcgcgcctgcttctccccgcccgggcgccagcgcccctgggcaggtgctgagcgccttctccgagcctcccctgctgcctccctcttccgcctagccgccttgctgctgcagtgcacatgggctgctggaggtagatgggctcaccgcccgtgaggcggcggtggatgcggcgctgggcagaaacagccaccgattccagctgccgtggggccgagcgcccggagcgccgctgcgagccccgggctcagccatggggacctccgcaagcagcatcaccgccctcgcctcttgcagccgcatcgccggccaagttggagccacgatggtcgccggctcccttctcttgcttgggttcctcagcaccatcacagcccaaccagaacaaaagactctgagtctcacgggcacgtaccgccacgttgaccgtaccactggccaggtgctaacctgcgacaagtgtccggcaggaacgtatgtctccgagcactgtaccaacacgagcctgcgagtctgcagcagctgcccctcggggacctttaccaggcatgagaacggcatagagagatgccatgactgtagtcagccatgcccacggccgatgattgagagattaccttgtgctgccttgactgaccgagaatgcatctgcccacccggaatgtatcagtctaatgggacctgcgctccccacacggtgtgccccgtgggctggggtgtgaggaagaaagggacagagaatgaagatgtgcggtgtaagcagtgtgctcgcggtaccttctctgacgtgccttccagtgtgatgaagtgtagagcccacacggactgtctgggtcagaacctgatggtggtcaagcaggggactaaggagacagacaacgtctgtggcgtgcacctgtcctcctccagcacgaccccatcttcccctggcatagctaccttttctcatcctgagcacacggaatcccacgatgtcccctcctccacctatgagccccaaggcatgaactcaacagattccaactctactgcctctgttagaactaaggtaccaagtgacatccaggaagagacagtgcctgacaatacaagctccacgagtgggaaggagagcacgaacaggaccctgccaaacccaccacaacttacccaccagcaaggcccccaccacagacacattctgaagctgctgccatcatccatggaggccactggtgagaagtccagcacagccatcaaggcccccaagaggggccatcccagacagaacccacacaagcatttcgacatcaacgagcatttgccctggatgattgtactcttccttctgctggtcctggtgctcattgtggtgtgcagtatccgaaagagctctaggactctcaagaaggggccccggcaggatcccagtgccatcatggaaaaggcagggctgaagaagtccttgactccaacccagaaccgggagaaatggatctactaccgcaatggccacggtattgacatcctgaagcttgtagcagcgcaggtgggaagccagtggaaggacatctatcagtttctttgcaacgccagcgagagggaggtggctgccttctccaatggatacactgcagaccatgaacgagcctatgcagctctgcagcactggaccatccggggccctgaggccagccttgctcagctcatcagcgccttgcgccagcaccgacgcaatgatgttgtggagaagattcgtgggctgatggaagatactacgcagttggaaaccgacaaactggctctccccatgagccccagtccgcttagcccgagccccattcccagtcctaacgtgaaacttgagaattccactctcctgacagtggagccctcacccctggacaagaacaagggcttcttcgtggacgagtcggagccccttctgcgttgtgactccacgtccagcggctcttcagcactgagcaggaatggctcctttataaccaaagaaaagaaggacacagtgttgcggcaggtccgcctggacccctgtgacttgcagcccatctttgacgacatgctgcacatcctgaatccggaagagctgcgggtaatcgaagagattccccaggctgaggacaaactggaccgtctcttcgagatcattggggtcaagagccaagaagccagccagaccctcttggactctgtgtacagccatcttcctgacctattgtagaacaccggggcactgcgttcaggaaatcagcctactggcagagtgatttcatttcttttctgacttttgtgttttggtgtgtgcgtgtatgtatgtgtttaacagagtatatggctggtgcttgagtttggggctctttctttctttctttctttctttccttctttctttcttttctcttcttgaaagtgaatgtataaagccttttcaatgtataactgttcgaaaatgcccaccactaaagttttttttttaagttccatatattctccatttttgccttcttatatattttcaacactattctgtgcactttaaaaacttaacatagacgcggtgtgacttttcccatatgctggattccaagactttgaacttcttaaaaaacataatggcatcttgtgacttctctaagtagacataagtctgccaacatcaacgcctactttgtctgttttaattatcactgctctctgtcgtattgtttgtccttcacgagcgtttctaaggacggagggaggctacgaccctgttgatgactgtaactctattagattttgagttgtcttttttcatgtcttgttctatagttcatattcacgacggaaacttgaccacactctctatcgctcgctgtatggttttcgtctggacatcgtacactgcttgtaacttgtgctccccttaatgctactaagctctgggctggagaatgaaatccttaagtccccaggacttgctgtttcagtggcttgacacctgggccaccaaagaactcgatcttcatcttttaggaacacctttgctgcaccttggaaaaccactttatgcccagctttgctttaaaagatatttttcttattttttttattcccctctctctctctctctctctctctctctctctctctctctctctctctctcaatacacatagtctataggtccagtctgccctcaaggccttgctgggtttcctttgtcatccaaccactttcgttaaaaatggctgcagctgtaagaactcctgtccgataaatttgcaactacgctctctcatttatcaacctgtcctctgatgctcagttgtcagactctaatgcagagctagacgcccactgcctttgtgtgggtgggcttagtggtgaggaactgatatcagaaaaatgccttcaagtatactaatttattaataaatattaggtgtttgttacttgcgtgattttggtgtgtttactacttaactttcccctttgcctctgttcaggttctagctctggtcttctcttggggaagaatgtgtgttctgaaagcctcctcctgcccaatctgtaaatcacacacattagcacaagtcccttttggagaggtacccggaggttcagtgcgcagggctttgataagaactgccaagcctttcctagtctttcgagttaaaccacaggcttttatttcctccaggtaatgttttttacctgtggggcaatctgcacacctccacagacttctagctaaaacctcagtgtccctgacgaactccagagggctttctcggcagtgaggcgggtgtagttttgtgttggtgggatttctcaagttcactgctggtttcctttaggaccccgcctctgcagaattccaatgactagacgctagcaattttaacttcctctttctgtgagaacctggaagtggatactgtatgcactgtgacgctcatgtcagcaactggggagtgtcttctggtttcattcctctccttgtggcagctttcaagaagaacaccccagtttcccttccctcttgttggtggaaaataagaactctgaggccaggtagtggtagtacatgcctttaatctattggaagagagaggcaggaggatattgagtttgaagccagcctggggagttccaggacatccatggctaccct

SEQ ID NO: 170 - protein, 655 aa, Rattus norvegicus
MGTSASSITALASCSRIAGQVGATMVAGSLLLLGFLSTITAQPEQKTLSLTGTYRHVDRTTGQVLTCDKCPAGTYVSEHCTNTSLRVCSSCPSGTFTRHENGIERCHDCSQPCPRPMIERLPCAALTDRECICPPGMYQSNGTCAPHTVCPVGWGVRKKGTENEDVRCKQCARGTFSDVPSSVMKCRAHTDCLGQNLMVVKQGTKETDNVCGVHLSSSSTTPSSPGIATFSHPEHTESHDVPSSTYEPQGMNSTDSNSTASVRTKVPSDIQEETVPDNTSSTSGKESTNRTLPNPPQLTHQQGPHHRHILKLLPSSMEATGEKSSTAIKAPKRGHPRQNPHKHFDINEHLPWMIVLFLLLVLVLIVVCSIRKSSRTLKKGPRQDPSAIMEKAGLKKSLTPTQNREKWIYYRNGHGIDILKLVAAQVGSQWKDIYQFLCNASEREVAAFSNGYTADHERAYAALQHWTIRGPEASLAQLISALRQHRRNDVVEKIRGLMEDTTQLETDKLALPMSPSPLSPSPIPSPNVKLENSTLLTVEPSPLDKNKGFFVDESEPLLRCDSTSSGSSALSRNGSFITKEKKDTVLRQVRLDPCDLQPIFDDMLHILNPEELRVIEEIPQAEDKLDRLFEIIGVKSQEASQTLLDSVYSHLPDLL